(19) United States
(12) Patent Application Publication
(10) Pub. No.: US 2020/0364801 A1
(43) Pub. Date: Nov. 19, 2020

(54) TRANSACTION ANALYSIS AND ASSET RECOVERY SYSTEM

(71) Applicant: Global Care Analytics, LLC, Cornelius, NC (US)

(72) Inventor: Guntaas Basra, Cornelius, NC (US)

(73) Assignee: Global Care Analytics, LLC, Cornelius, NC (US)

(21) Appl. No.: 16/942,400

(22) Filed: Jul. 29, 2020

Related U.S. Application Data
(63) Continuation-in-part of application No. PCT/US2020/037823, filed on Jun. 15, 2020.
(60) Provisional application No. 62/833,841, filed on Apr. 15, 2019.

(51) Int. Cl. G06Q 40/08; G06Q 10/10; G16H 40/20; G06Q 50/18; G16H 70/20; G16H 10/60; G06Q 30/00; G06Q 40/00; G06Q 30/04; G06N 20/00; G06N 5/04; G06F 16/25; G06K 9/00; G06F 40/205; G06F 40/40; H04L 29/08

(52) U.S. Cl. CPC G06Q 40/08; G06K 2209/01; G16H 40/20; G06Q 50/18; G16H 70/20; G16H 10/60; G06Q 30/0185; G06Q 40/12; G06Q 30/04; G06N 20/00; G06N 5/04; G06F 16/258; G06K 9/00442; G06F 40/205; G06F 40/40; H04L 67/2809; G06Q 10/10

(57) ABSTRACT

A transaction analysis and asset recovery system can include interfaces for inputs and outputs, a contract ingestion engine, a transaction automation engine, and a payment reconciliation engine. Inputs can include contract data regarding transaction pricing and transaction data regarding specific transactions and related payments made. The contract ingestion engine can receive, normalize, and parse the contract data, and populate a contract rules database with the parsed contract data. The transaction automation engine can receive, normalize, and parse the transaction data, and populate a transaction instances database with the parsed transaction data. The payment reconciliation engine can receive parsed transaction data, correlate the received parsed transaction data with appropriate parsed contract data, compare payments made in the received parsed transaction data with transaction pricing in the correlated parsed contract data, and flag discrepancies between the payments made and the correlated transaction pricing. Flagged discrepancies can include recoverable assets.

### CROSS-REFERENCE TO RELATED APPLICATION

The present application claims benefit, pursuant to the provisions of 35 U.S.C. § 119, of U.S. Provisional Application Ser. No. 62/833,841, titled “SYSTEM FOR TRANSACTION ANALYTICS”, naming Basra et al. as inventors, and filed 15 Apr. 2019, the entirety of which is incorporated herein by reference for all purposes.

The present Application is a Continuation-in-Part of pending International Patent Application PCT/US20/37823, titled “TRANSACTION ANALYSIS AND ASSET RECOVERY SYSTEM”, naming Basra as inventors, filed on Jun. 15, 2020, and designating the United States, the entirety of which is incorporated herein by reference for all purposes.

### TECHNICAL FIELD

The present invention relates generally to transaction analytics, and more particularly to the identification of discrepancies in billing and payment transactions.

### BACKGROUND

To prevent lost revenues and manage costs, various service providers and other vendors need to deal with billings and payment collections using increasingly complex information technology services. As large volumes of data packets now pass through provider and partner networks, many service providers and vendors struggle to accurately track, aggregate, and interpret enormous numbers of individual transactions. These issues can be compounded where third party payors are responsible for paying the providers. As such, providers frequently fail to capture full value from the services and goods that they provide. Historical experience and analysis find that these providers may unknowingly be losing hundreds of thousands of dollars of revenue every month.

Traditionally, transaction events are fed to a reporting data warehouse from a mediation system. This approach is often based upon batched and scheduled processing, however, and real-time or near real-time processing is typically not an option. In addition to this inherent drawback, using a reporting data warehouse approach also requires a larger hardware investment to scale for the processing spikes of each batch while leaving large amounts of idle or low load time. Changes to the reporting data warehouse to support changing business models are usually slow and simplified to support the broadest possible constituent base and are often dependent upon the ability to fit new processing requirements into existing windows of opportunity.

Analytic reports generated from the reporting data warehouse typically use data query relationships and constraints that are not well preserved for detailed auditing and forensics. Missing data is common, and it can be very difficult to recapture or otherwise determine this missing data in the reporting data warehouse. In addition, many current transaction analysis auditing systems and methods typically rely very heavily on manual, decentralized process knowledge and labor-intensive processes that do not scale for finding anomalies in mass quantities of service transactions across regions and markets Again, this results in providers losing a tremendous amount of revenue over time.

Although traditional manual transactional analysis and auditing systems and methods have worked well in the past, improvements are still desirable. In particular, what is desired are automated transaction analysis and asset recovery systems that are able to analyze massive amounts of transactions across many providers and payors to recover lost assets and establish improved rules for analyzing past, current, and future transactions.

### BRIEF DESCRIPTION OF THE DRAWINGS

The included drawings are for illustrative purposes and serve only to provide examples of possible structures and arrangements for the disclosed inventive apparatuses, systems and methods for transaction analysis and asset recovery. These drawings in no way limit any changes in form and detail that may be made to the disclosure by one skilled in the art without departing from the spirit and scope of the disclosure.

FIG. 1A illustrates in block diagram format an exemplary computerized data network according to one embodiment of the present disclosure.

FIG. 1B illustrates in front perspective view an exemplary computing device according to one embodiment of the present disclosure.

FIG. 1C illustrates in front perspective view an alternative exemplary computing device according to one embodiment of the present disclosure.

FIG. 2 illustrates a diagram of an exemplary client or end user computing device or system according to one embodiment of the present disclosure.

FIG. 3 illustrates in block diagram format an exemplary client system for a mobile device according to one embodiment of the present disclosure.

FIG. 4 illustrates in block diagram format an exemplary server system according to one embodiment of the present disclosure.

FIG. 5 illustrates in block diagram format an exemplary application server system according to one embodiment of the present disclosure.

FIG. 6 illustrates a flowchart of an exemplary high level overall method of analyzing transactions using a computing system according to one embodiment of the present disclosure.

FIG. 7 illustrates in block diagram format an exemplary overall computing system for analyzing transactions and recovering assets according to one embodiment of the present disclosure.

FIG. 8 illustrates in block diagram format an exemplary back end portion of a computing system for analyzing transactions and recovering assets according to one embodiment of the present disclosure.

FIG. 9 illustrates a flowchart of an exemplary method of processing contract data using a Contract Ingestion Engine of a computing system according to one embodiment of the present disclosure.

FIG. 10A illustrates an exemplary portion of contract data being parsed by a Contract Ingestion Engine of a computing system according to one embodiment of the present disclosure.

FIG. 10B illustrates an exemplary portion of parsed and reorganized contract data by a Contract Ingestion Engine of a computing system according to one embodiment of the present disclosure.

FIG. 10C illustrates an exemplary portion of contract rules stored on a Contract Rules Database by a Contract Ingestion Engine of a computing system according to one embodiment of the present disclosure.

FIG. 10D illustrates another exemplary portion of contract rules stored on a Contract Rules Database by a Contract Ingestion Engine.

FIG. 11 illustrates a flowchart of an exemplary method of processing claim data using a Claim Automation Engine of a computing system according to one embodiment of the present disclosure.

FIG. 12 illustrates a flowchart of an exemplary method of normalizing claim data using a Claim Automation Engine of a computing system according to one embodiment of the present disclosure.

FIG. 13A illustrates an exemplary portion of claims data being normalized and organized by a Claim Automation Engine of a computing system according to one embodiment of the present disclosure.

FIG. 13B illustrates an exemplary portion of parsed and reorganized claims data by a Claim Automation Engine of a computing system according to one embodiment of the present disclosure.

FIG. 13C illustrates an exemplary portion of claim records stored on a Claims Database by a Claims Automation Engine of a computing system according to one embodiment of the present disclosure.

FIG. 14 illustrates a flowchart of an exemplary method of analyzing transaction data using a Payment Reconciliation Engine of a computing system according to one embodiment of the present disclosure.

FIG. 15 illustrates a flowchart of an exemplary method of recovering lost assets found using a Payment Reconciliation Engine of a computing system according to one embodiment of the present disclosure.

FIG. 16 illustrates a flowchart of an exemplary method of creating a new system rule using a Payment Reconciliation Engine of a computing system according to one embodiment of the present disclosure.

FIG. 17 illustrates a timing diagram of an exemplary new rule message queue for use by a Payment Reconciliation Engine and associated message brokers of a computing system according to one embodiment of the present disclosure.

FIGS. 18A-18D illustrate flowcharts of an exemplary detailed overall method of analyzing transactions using a computing system using system engines, modules, and components that may operate asynchronously and substantially concurrently according to one embodiment of the present disclosure.

FIGS. 19-55 illustrate screenshots of an exemplary GUI that may be used to facilitate, initiate and/or perform various operation(s) and/or action(s) relating to the disclosed transaction analysis and asset recovery systems and overall Transaction Analysis Procedure according to various embodiments of the present disclosure.

### DETAILED DESCRIPTION OF EXAMPLE EMBODIMENTS

### Overview

It is an advantage of the present disclosure to provide new transaction analysis and asset recovery systems that are able to analyze massive amounts of transactions across many providers and payors to recover lost assets and establish improved rules for analyzing transactions. This can be accomplished at least in part through the use of innovative automated computing systems that are hosted by a transaction analytics and asset recovery entity over a network. In particular, such transaction analysis and asset recovery systems and methods can include various specialized engines that can rapidly normalize, parse, analyze, and provide reports on massive amounts of data from many sources and in a wide variety of different original formats.

Various aspects described or referenced herein are directed to different methods, systems, and computer program products for implementing and performing transaction analysis and asset recovery operations relating to billing transactions between healthcare providers and insurance company payors.

One aspect disclosed herein is directed to different methods, systems, and computer program products for analyzing transactions between healthcare providers and insurance company payors. In at least one embodiment, various method(s), system(s) and/or computer program product(s) may be operable to cause at least one processor to execute a plurality of instructions stored in non-transient memory to: cause a contract ingestion engine to access a first contract data file, the first contract data file including contract information identifying a first healthcare provider and a first insurance payor; cause the contract ingestion engine to automatically process the first contract data file using optical character recognition, machine learning, and natural language processing; wherein the processing of the first contract data file includes parsing the first contract data file to identify a first set of medical procedure identifier parameters identifying a first medical procedure; wherein the processing of the first contract data file further includes parsing the first contract data file to identify a first set of contract payment rate parameters relating to the first medical procedure; cause the contract ingestion engine to generate, using the processed data of the first contract data file, a first contract rule record which includes the first set of medical procedure identifier parameters and includes the first set of contract payment rate parameters; cause the contract ingestion engine to populate a first contract rules database with information relating to the first contract rule, including the first set of medical procedure identifier parameters and the first set of contract payment rate parameters; cause a claim automation engine to access a first claim data file the first claim data file including a first portion of claim information identifying a first claim record, the first claim record including: a healthcare provider identifier identifying a second healthcare provider, an insurance payor identifier identifying a second insurance payor, and a patient identifier identifying a first patient; cause the claim automation engine to process the first portion of claim information to identify the healthcare provider identifier, the insurance payor identifier, and the patient identifier; wherein the first portion of claim information includes a first portion of medical procedure identifier data identifying a first performed medical procedure associated with the first claim record; the first portion of claim information further including a first portion of Payor paid amount data relating to a first Payor paid amount in connection with the first performed medical procedure; cause the claim automation engine to generate, using the processed first portion of claim information, a first processed claim record which includes normalized information identifying: the healthcare provider identifier, the insurance payor identifier, the patient identifier, the first performed medical procedure, and the first Payor paid amount; cause the claim automation engine to populate a first claims database with the first processed claim record; cause a payment reconciliation engine to access a second set of contract rule parameters associated with a second contracts rule, the second set of contract rule parameters including a second set of medical procedure identifier parameters identifying a second medical procedure, the second set of contract rule parameters further including a second set of contract payment rate parameters relating to the second medical procedure; cause the payment reconciliation engine to access information relating to a second processed claim record, the second processed claim record including a second portion of medical procedure identifier data identifying a second performed medical procedure associated with the second processed claim record, the second processed claim record further including a second portion of Payor paid amount data relating to a second Payor paid amount in connection with the second performed medical procedure; cause the payment reconciliation engine to determine, using the second set of contract rule parameters and the second processed claim record information, whether the second performed medical procedure associated with the second processed claim record is affected by the second set of contract rule parameters; payment reconciliation engine automation engine to calculate, in response to determining that the second performed medical procedure is affected by the second set of contract rule parameters, an expected contract payment amount for the second processed claim record, wherein the expected contract payment amount is calculated using the second set of contract rule parameters and the second processed claim record information; cause the payment reconciliation engine to compare the expected contract payment amount to the second Payor paid amount; cause the payment reconciliation engine to determine whether any payment discrepancy is detected between the expected contract payment amount and the second Payor paid amount; cause the payment reconciliation engine to generate, in response to detecting at least one payment discrepancy between the expected contract payment amount and the second Payor paid amount, a first portion of claim discrepancy data which includes information identifying the second processed claim record and includes claim payment discrepancy information relating to the at least one detected payment discrepancy associated with the second processed claim record; and cause the payment reconciliation engine to automatically initiate, in response to detecting at least one payment discrepancy between the expected contract payment amount and the second Payor paid amount, at least one operation to facilitate resolution of the at least one detected payment discrepancy associated with the second processed claim record.

Additional method(s), system(s) and/or computer program product(s) may be further operable to cause at least one processor to execute additional instructions to: monitor the first contract rules database for at least one rule update event, the at least one rule update event relating to an occurrence of a new or modified rule record at the first contract rules database; detect a rule update event relating to an identified contract rule record in the first contract rules database; analyze the identified contract rule record to identify a set of contract rule parameters associated with the identified contract rule record; and automatically instantiate and configure at least one automated agent process to perform payment reconciliation analysis on a first set of claim records using the identified set of contract rule parameters.

Additional method(s), system(s) and/or computer program product(s) may be further operable to cause at least one processor to execute additional instructions to: cause the contract ingestion engine to populate a contract rules database with normalized information relating to the first contract rule.

Additional method(s), system(s) and/or computer program product(s) may be further operable to cause at least one processor to execute additional instructions to convert data from electronic data interchange (“EDI”) files to converted data of a different format. In at least one embodiment, the converted data is in JavaScript Object Notation (“JSON”) format.

Additional method(s), system(s) and/or computer program product(s) may be further operable to cause at least one processor to execute additional instructions to access a second contract data file, the second contract data file including contract information identifying a second healthcare provider and a second insurance company payor, wherein the first contract data file and the second contract data file are of disparate file formats.

Additional method(s), system(s) and/or computer program product(s) may be further operable to cause at least one processor to execute additional instructions to: detect a rule update event relating to an identified contract rule record in a first contract rules database; analyze the identified contract rule record to identify a third set of contract rule parameters associated with the identified contract rule record, the third set of contract rule parameters including a third set of medical procedure identifier parameters identifying a third medical procedure, the third set of contract rule parameters further including a third set of contract payment rate parameters relating to the third medical procedure; automatically instantiate and configure at least one automated agent process to perform payment reconciliation analysis on a third set of claim records using the third set of contract rule parameters; wherein the payment reconciliation analysis includes: accessing information relating to a third processed claim record, the third processed claim record including a third portion of medical procedure identifier data identifying a third performed medical procedure associated with the third processed claim record, the third processed claim record further including a third portion of Payor paid amount data relating to a third Payor paid amount in connection with the third performed medical procedure; determining, using the third set of contract rule parameters and the third processed claim record information, whether the third performed medical procedure associated with the third processed claim record is affected by the third set of contract rule parameters; calculating, in response to determining that the third performed medical procedure is affected by the third set of contract rule parameters, a second expected contract payment amount for the third processed claim record, wherein the second expected contract payment amount is calculated using the third set of contract rule parameters and the third processed claim record information; comparing the second expected contract payment amount to the third Payor paid amount; determining whether any payment discrepancy is detected between the second expected contract payment amount and the third Payor paid amount; and generating, in response to detecting at least one payment discrepancy between the second expected contract payment amount and the third Payor paid amount, a second portion of claim discrepancy data which includes information identifying the third processed claim record and includes claim payment discrepancy information relating to the at least one detected payment discrepancy associated with the third processed claim record.

Additional method(s), system(s) and/or computer program product(s) may be further operable to cause at least one processor to execute additional instructions to: detect a first rule update event relating to a first identified contract rule record in a first contract rules database; analyze the first identified contract rule record to identify a first set of contract rule parameters associated with the first identified contract rule record; automatically instantiate and configure a first automated agent process to perform a first set payment reconciliation analysis operations on a first set of claim records using the first identified set of contract rule parameters; detect a second rule update event relating to a second identified contract rule record in a second contract rules database; analyze the second identified contract rule record to identify a second set of contract rule parameters associated with the second identified contract rule record; automatically instantiate and configure a second automated agent process to perform a second set payment reconciliation analysis operations on a second set of claim records using the second identified set of contract rule parameters; and wherein the first set payment reconciliation analysis operations and second set payment reconciliation analysis operations are performed substantially asynchronously and substantially concurrently. In at least one embodiment, new claim data added to the system after creation of the first contract rule may be processed according to the first contract rule.

Additional method(s), system(s) and/or computer program product(s) may be further operable to cause at least one processor to execute additional instructions to separate flagged discrepancies by payor; place the separated flagged discrepancies into categories, wherein the categories include one or more of denials, underpayments, unique short pays, systemic discrepancies of the same error type, or overcollections, and; rebill each payor according to its respective flagged discrepancies. In at least one embodiment, a significant number of rebilled flagged discrepancies are less than about $1000.

Additional method(s), system(s) and/or computer program product(s) may be further operable to cause at least one processor to execute additional instructions to automatically processes at least 50 million claim data records per hour for claim payment analysis and reconciliation.

Additional method(s), system(s) and/or computer program product(s) may be further operable to cause at least one processor to execute additional instructions to create and read existing contract rules when any changes occur, and store the newly created contract rules in a contract rules database.

Additional method(s), system(s) and/or computer program product(s) may be further operable to cause at least one processor to execute additional instructions to generate a message to trigger a new process when a new contract rule is created, wherein the new process includes a stored function performable by automated agents, place the message in a message queue, and; deliver the message to a third automated agent using a message broker when the message matures through the message queue, wherein the third automated agent is configured to execute instructions for performing a third payment reconciliation analysis involving the new contract rule.

Other aspects disclosed herein are directed to different methods, systems, and computer program products for performing transaction analysis and payment reconciliation relating to billing transactions between healthcare providers and insurance company payors. In at least one embodiment, the various techniques described herein may be implemented at one or more computer-based transaction analysis and asset recovery system(s) comprising: one or more interfaces configured to receive inputs and provide outputs to a user, wherein the received inputs include contract data regarding transaction pricing and transaction data regarding specific transactions and related payments made; a contract ingestion engine coupled to at least one of the one or more interfaces, wherein the contract ingestion engine is configured to import the contract data, normalize the imported contract data, parse the normalized contract data, and populate a contract rules database with the parsed contract data; a transaction automation engine coupled to at least one of the one or more interfaces, wherein the transaction automation engine is configured to receive the transaction data, normalize the received transaction data, parse the normalized transaction data, and populate a transactions database with the parsed transaction data; and a payment reconciliation engine coupled to at least one of the one or more interfaces, wherein the payment reconciliation engine is configured to receive parsed transaction data from the transaction instances database, correlate the received parsed transaction data with appropriate parsed contract data in the contract rules database, compare payments made in the received parsed transaction data with transaction pricing in the correlated parsed contract data, flag discrepancies between the payments made and the correlated transaction pricing, generate claim discrepancy data including at least claim identifications and discrepancy amounts, and provide access to the claim discrepancy data to at least one entity to facilitate recovery of the discrepancy amount.

In at least one embodiment, the transaction analysis and asset recovery system may include the contract rules database and the transactions database. In at least one embodiment, the transaction data includes service providers, service receivers, services performed, responsible payors, and payments made. In at least one embodiment, the payment reconciliation engine is further configured to create a new rule when a discrepancy is flagged for which no rule exists and store the newly created rule in the contract rules database. In at least one embodiment, the payment reconciliation engine is further configured to generate a message to trigger a new process when the new rule is created, the new process including comparing payments made in the received parsed transaction data with correlated transaction pricing in the parsed contract data for claims affected by the new rule, placing the message in a message queue, delivering the message using a message broker when the message matures through the message queue, and performing the new process asynchronously and substantially concurrently while processing new transaction data according to the new rule, the new transaction data being added to the system after creation of the new rule.

Other aspects disclosed herein are directed to different methods, systems, and computer program products for performing transaction analysis relating to billing transactions between healthcare providers and insurance company payors. In at least one embodiment, various method(s), system(s) and/or computer program product(s) may be operable to cause at least one processor to execute a plurality of instructions stored in non-transient memory to: import contract data from a plurality of contracts between service providers and responsible payors; normalize the imported contract data into a format that is readily usable by the computing system; parse the normalized contract data into specific contract data portions that are readily usable by the computing system; populate a contract rules database with the parsed contract data; receive transaction data in a plurality of different formats from a plurality of different service providers, wherein the transaction data includes service providers, service receivers, services performed, responsible payors, and payments made; normalize the received transaction data into a format that is readily usable by the computing system; parsing the normalized transaction data into specific transaction data portions that are readily usable by the computing system; populate a transaction instances database with the parsed transaction data; correlate a set of specific transaction data portions with appropriate specific contract data portions; compare payments made in the set of specific transaction data portions with transaction pricing in the correlated specific contract data portions; flag discrepancies between the payments made and the correlated transaction pricing, wherein at least some of the flagged discrepancies include recoverable assets; generate claim discrepancy data including at least claim identifications and discrepancy amounts; and provide access to the claim discrepancy data to at least one entity to facilitate recovery of the discrepancy amounts.

Additional method(s), system(s) and/or computer program product(s) may be further operable to cause at least one processor to execute additional instructions to: create a new rule when a discrepancy is flagged for which no rule exists; store the newly created rule in the contract rules database; generate a message to trigger a new process when the new rule is created, wherein the new process includes compare payments made in the parsed transaction data with correlated transaction pricing in the parsed contract data for all claims affected by the new rule; place the message in a message queue; deliver the message using a message broker when the message matures through the message queue; and perform the new process asynchronously and simultaneously while processing new transaction data according to the new rule, wherein the new transaction data has been added to the computing system after creation of the new rule.

Additional aspects disclosed herein are directed to different methods, systems, and computer program products for analyzing transactions between healthcare providers and insurance company payors. In at least one embodiment, various method(s), system(s) and/or computer program product(s) may be operable to cause at least one processor to execute a plurality of instructions stored in non-transient memory to: cause a contract ingestion engine to access a first contract data file, the first contract data file including contract information identifying a first healthcare provider and a first insurance payor; cause the contract ingestion engine to automatically process the first contract data file using optical character recognition, machine learning, and natural language processing; wherein the processing of the first contract data file includes parsing the first contract data file to identify a first set of medical procedure identifier parameters identifying a first medical procedure; wherein the processing of the first contract data file further includes parsing the first contract data file to identify a first set of contract payment rate parameters relating to the first medical procedure; cause the contract ingestion engine to generate, using the processed data of the first contract data file, a first contract rule record which includes the first set of medical procedure identifier parameters and includes the first set of contract payment rate parameters; cause the contract ingestion engine to populate a first contract rules database with information relating to the first contract rule, including the first set of medical procedure identifier parameters and the first set of contract payment rate parameters; cause a claim automation engine to access a first claim data file the first claim data file including a first portion of claim information identifying a first claim record, the first claim record including: a healthcare provider identifier identifying a second healthcare provider, an insurance payor identifier identifying a second insurance payor, and a patient identifier identifying a first patient; cause the claim automation engine to process the first portion of claim information to identify the healthcare provider identifier, the insurance payor identifier, and the patient identifier; wherein the first portion of claim information includes a first portion of medical procedure identifier data identifying a first performed medical procedure associated with the first claim record; the first portion of claim information further including a first portion of Provider billed amount data relating to a first Provider billed amount in connection with the first performed medical procedure; the first portion of claim information further including a first portion of Payor paid amount data relating to a first Payor paid amount in connection with the first performed medical procedure; cause the claim automation engine to generate, using the processed first portion of claim information, a first processed claim record which includes normalized information identifying: the healthcare provider identifier, the insurance payor identifier, the patient identifier, the first performed medical procedure, the first Provider billed amount, and the first Payor paid amount; cause the claim automation engine to populate a first claims database with the first processed claim record; cause a payment reconciliation engine to access a second set of contract rule parameters associated with a second contracts rule, the second set of contract rule parameters including a second set of medical procedure identifier parameters identifying a second medical procedure, the second set of contract rule parameters further including a second set of contract payment rate parameters relating to the second medical procedure; cause the payment reconciliation engine to access information relating to a second processed claim record, the second processed claim record including a second portion of medical procedure identifier data identifying a second performed medical procedure associated with the second processed claim record, the second processed claim record further including a second portion of Provider billed amount data relating to a second Provider billed amount in connection with the second performed medical procedure, the second processed claim record further including a second portion of Payor paid amount data relating to a second Payor paid amount in connection with the second performed medical procedure; cause the payment reconciliation engine to determine, using the second set of contract rule parameters and the second processed claim record information, whether the second performed medical procedure associated with the second processed claim record is affected by the second set of contract rule parameters; payment reconciliation engine automation engine to calculate, in response to determining that the second performed medical procedure is affected by the second set of contract rule parameters, an expected contract payment amount for the second processed claim record, wherein the expected contract payment amount is calculated using the second set of contract rule parameters and the second processed claim record information; cause the payment reconciliation engine to compare the expected contract payment amount to the second Payor paid amount; cause the payment reconciliation engine to determine whether any payment discrepancy is detected between the expected contract payment amount and the second Payor paid amount; cause the payment reconciliation engine to generate, in response to detecting at least one payment discrepancy between the expected contract payment amount and the second Payor paid amount, a first portion of claim discrepancy data which includes information identifying the second processed claim record and includes claim payment discrepancy information relating to the at least one detected payment discrepancy associated with the second processed claim record; and cause the payment reconciliation engine to automatically initiate, in response to detecting at least one payment discrepancy between the expected contract payment amount and the second Payor paid amount, at least one operation to facilitate resolution of the at least one detected payment discrepancy associated with the second processed claim record.

Other apparatuses, systems, methods, features and advantages of the disclosure will be or will become apparent to one with skill in the art upon examination of the following figures and detailed description. It is intended that all additional apparatuses, systems, methods, features and advantages be included within this description, be within the scope of the disclosure, and be protected by the accompanying claims

### Specific Example Embodiments

Exemplary applications of apparatuses and methods according to the present disclosure are described in this section. These examples are being provided solely to add context and aid in the understanding of the disclosure. It will thus be apparent to one skilled in the art that the present disclosure may be practiced without some or all of these specific details. In other instances, well known process steps have not been described in detail in order to avoid unnecessarily obscuring the present disclosure. Other applications are possible, such that the following examples should not be taken as limiting.

In the following detailed description, references are made to the accompanying drawings, which form a part of the description and in which are shown, by way of illustration, specific embodiments of the present disclosure. Although these embodiments are described in sufficient detail to enable one skilled in the art to practice the disclosure, it is understood that these examples are not limiting, such that other embodiments may be used, and changes may be made without departing from the spirit and scope of the disclosure.

The present disclosure relates in various embodiments to new transaction analysis and asset recovery systems and methods that are able to analyze massive amounts of complex transactions across many providers and payors to recover lost assets and establish improved rules for analyzing past, current, and future transactions. In particular, the disclosed systems and methods identify errors in billing and transactions practices, provide system rules based on the identified errors where appropriate, and provide predictive or forward-looking remedies so that transaction errors can be corrected not only so that assets may be recouped, but also so that future errors do not create additional errors and asset losses in transactions.

The disclosed transaction analysis and asset recovery systems and methods can include specialized engines that rapidly normalize, parse, and analyze massive amounts of data from many sources and in a wide variety of different original formats, provide reports and helpful review tools for users based on the processed data, establish new rules based on errors found in the data, and assist in recovering lost assets due to billing and payment errors found in the data. In various embodiments, the disclosed systems can process over 50 million complex transactions per hour, whereas prior techniques would need at least 24 to 48 hours to process those same transactions.

In various embodiments, a system can take several years (e.g., five years) of past data from a service providing organization, process the data, and then rebill the analyzed data properly as prescribed by established contracts between the service providing organization and respective payors. When an error or issue with a payment or transaction is recognized, the system can log specifics about a mis-pay or other problem with a billing event, such as the payor, the type of bill, the payment discrepancy, and other relevant details. An identified payment error can be assigned a specific issue code, and the system can also access and provide a transaction code for each past transaction.

The system can examine definitions and requirements from actual contracts and compare these to what was actually paid across thousands or millions of transactions. If there is a difference, the system can uncover reasons for the issue and link issue codes to payor transaction codes when identifying transactions that have been paid incorrectly. Similar mis-pays or other problems can then be put into specific groups. Some groups can be automatically adjudicated and paid electronically without any human oversight. Other groups may require a rebilling to a payor in order for the payor to review and possibly contest the changes. Still other groups may require an appeal process, and the system can assist with forming such appeals.

In various specific arrangements, disclosed systems can relate to transactions or “claims” between health care service providers and insurance payors, for example. After the system processes past claim and payment data in such arrangements, a detailed report may give a comprehensive analysis of each contract that the payor had, such as, for example between a payor and a hospital or physician group. A report may identify, for example, three different types of claim recoveries. These can include Normal Mis-Pays or Short-Pays, which are claims that were properly billed but were found to be erroneously written off by the insurance company or responsible payor as “zero-balance claims.” These Mis-Pay claims can simply be resubmitted to the payor for recovery and payment. Another type of claim recovery can be termed Projects, which can involve a large systemic issue across many claims These Project claims can be batched together as one project, which can then lead to a settlement negotiation with the payor. Still another type of claim recovery can be termed Over-Collections, which can involve an underbilled error on the part of the healthcare provider. These claims can simply be rebilled at the proper rate. Other types of claim recoveries may also be possible, and various system arrangements relating to the specifics of analyzing healthcare provider and insurance payor billing and payment issues are provided in greater detail below.

While the various systems and methods disclosed herein refer to the specific arrangements of healthcare providers and insurance payors for purposes of illustration, it will be appreciated that other industries that utilize service provider to payor arrangements that involve contracts and regular billing and payments under those contracts may also make use of the various disclosed systems and methods. Such arrangements can include, for example, building construction contracting and subcontracting, labor procurement and delivery, commodities trading, supply chain and logistics, and media advertising, as well as any other alternative industry and relational arrangement with high volume transactions based on contractual obligations. Various adjustments and extrapolations to the disclosed systems and methods can be made as appropriate to such alternative arrangements, as will be readily appreciated by those of skill in the art.

### Exemplary Computing Systems

As will be readily appreciated, the disclosed systems and methods require the use of a sophisticated computing system that can be distributed across multiple locations and entities. The computing system can include a user-friendly front end that has input output and graphical user interface software, as well as a processing heavy backend that includes large databases and complex engines that access the databases. These various items can be provided on a computing system in a wide variety of ways, as will be readily appreciated by one of skill in the art. While the exemplary computing systems and components thereof provided below may be one way of implementing the functions of the disclosed systems and methods, many other types of computing systems and alternative components may also be used, and the illustrative examples provided in this section shall not be limiting in any way with respect to the implementation of the disclosed systems.

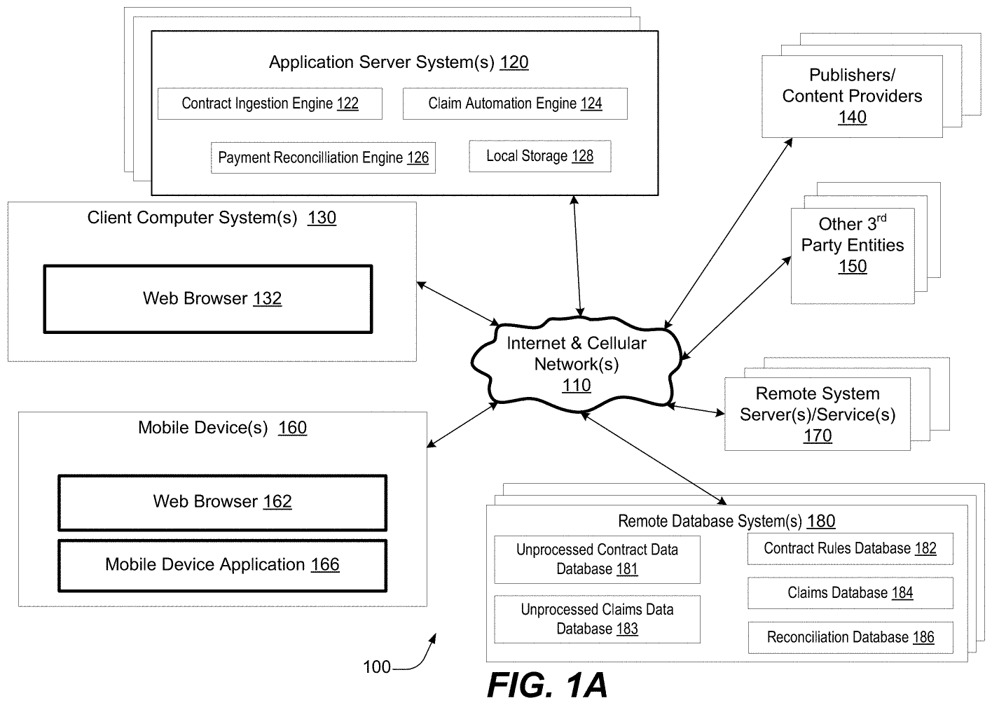

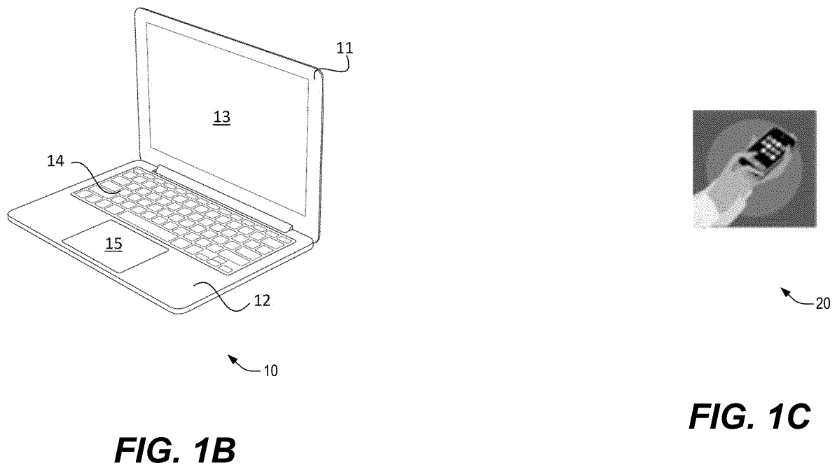

Referring first to FIG. 1A, an exemplary computerized data network according to one embodiment of the present disclosure is shown in block diagram format. Computerized data network 100 can be used to implement a “Transaction Analytics System” adapted for transaction analysis and asset recovery, for example. As described in greater detail herein, different embodiments of Transaction Analytics Systems may be configured, designed, and/or operable to provide various different functions regarding past, present, and future transactions. Further, as described in greater detail herein, many of the various operations, functionalities, and/or features of the disclosed Transaction Analytics System may enable or provide different types of advantages and/or benefits to different entities interacting with the Transaction Analytics System.

According to different embodiments, at least some Transaction Analytics System may be configured, designed, and/or operable to provide a number of different advantages and/or benefits and/or may be operable to initiate, and/or enable various different types of operations, functionalities, and/or features, According to different embodiments, at least a portion of the various types of functions, operations, actions, and/or other features provided by the Transaction Analytics System may be implemented at one or more client systems, at one or more server systems, and/or combinations thereof.

According to various embodiments, the computerized data network 100 adapted for implementation of a Transaction Analytics System may include a plurality of different types of components, devices, modules, processes, systems, etc., which, for example, may be implemented and/or instantiated via the use of hardware and/or combinations of hardware and software. For example, as illustrated in the example embodiment of FIG. 1A, network 100 may include one or more of the following types of systems, components, devices, processes, etc. (or combinations thereof):
Application Server System(s) 120—In at least one embodiment, the Application Server System(s) may be operable to perform and/or implement various types of functions, operations, actions, and/or other features such as those described or referenced herein. In particular, Application Server System(s) 120 can include at least a Contract Ingestion Engine 122, a Claim Automation Engine 124, and a Payment Reconciliation Engine 126, each of which are described in greater detail below. Application Server System(s) 120 and/or each of its individual engines may have localized storage, such as Local Storage 128, and may also be coupled to one or more components from Remote Database System(s) 180. Application Server System(s) 120 and/or each of its individual engines may also monitor Remote Database System(s) and/or its individual databases for activity, upon which action may be take, as set forth in greater detail below.
Publisher/Content Provider System component(s) 140
Client Computer System (s) 130
3^{rd} Party System(s) 150
Internet & Cellular Network(s) 110
Remote Database System(s) 180—In at least one embodiment, the Remote Database System(s) may be operable to store data for various system components and implement other features such as those described or referenced herein. In particular, the Remote Database System(s) can include at least an Unprocessed Contract Data Database 181, a Contract Rules Database 182, an Unprocessed Claims Data Database, 183, a Claims Database 184, and a Reconciliation Database 186, each of which are described in greater detail below. In some arrangements, Application Server System(s) 120 and/or each of its individual engines may monitor Unprocessed Contract Data Database 181 and/or Unprocessed Claims Data Database for new activity and then act accordingly when new activity occurs. Other databases may also be possible These different databases may be physically accessible to an operator or user and/or may cloud based, and each of these different databases may be distributed and/or may not necessarily be together in the same location.
Remote Server System(s)/Service(s)170, which, for example, may include, but are not limited to, one or more of the following (or combinations thereof):
   Content provider servers/services
   Media Streaming servers/services
   Database storage/access/query servers/services
   Financial transaction servers/services
   Payment gateway servers/services
   Electronic commerce servers/services
   Event management/scheduling servers/services
Mobile Device(s) 160—In at least one embodiment, the Mobile Device(s) may be operable to perform and/or implement various types of functions, operations, actions, and/or other features such as those described or referenced herein.

In some embodiments, a decentralized server system may be used for a Transaction Analytics System. Rather than utilizing a centralized server, various system functions can be performed at multiple servers distributed at different locations across a distributed network. As such, while Application Server System(s) 120 can all be on one machine or placed at a single location, Application Server System(s) 120 may also be on multiple machines at multiple locations. For example, one portion of Application Server System(s) 120 relating to contract intake and contract data processing may be located on a first server at one location, while another portion of Application Server System(s) 120 relating to claim instances intake may be located on a second server located at another location, while still another portion of Application Server System(s) 120 relating to transaction analysis may be located on a third server located at still another location. Communications between the different portions of Application Server System(s) 120 may be arranged as appropriate to facilitate functionality between the different system portions.

In at least one embodiment, a Transaction Analytics System may be operable to utilize and/or generate various different types of data and/or other types of information when performing specific tasks and/or operations. This may include, for example, input data/information and/or output data/information. For example, in at least one embodiment, a Transaction Analytics System may be operable to access, process, and/or otherwise utilize information from one or more different types of sources, such as, for example, one or more local and/or remote memories, devices and/or systems. Additionally, in at least one embodiment, a Transaction Analytics System may be operable to generate one or more different types of output data/information, which, for example, may be stored in memory of one or more local and/or remote devices and/or systems. Examples of different types of input data/information and/or output data/information which may be accessed and/or utilized by a Transaction Analytics System may include, but are not limited to, one or more of those described and/or referenced herein.

According to specific embodiments, multiple instances or threads of a Transaction Analytics System may be concurrently implemented and/or initiated via the use of one or more processors, software engines, modules, and/or other combinations of hardware and/or hardware and software. In at least some embodiments, various aspects, features, and/or functionalities of the Transaction Analytics System may be performed, implemented and/or initiated by one or more of the various systems, components, systems, devices, procedures, processes, etc., described and/or referenced herein.

In at least one embodiment, a given instance of a Transaction Analytics System may access and/or utilize information from one or more associated databases. In at least one embodiment, at least a portion of the database information may be accessed via communication with one or more local and/or remote memory devices. Examples of different types of data which may be accessed by a Transaction Analytics System may include, but are not limited to, one or more of those described and/or referenced herein.

According to different embodiments, various different types of encryption/decryption techniques may be used to facilitate secure communications between devices in a Transaction Analytics System and/or other networks. Examples of the various types of security techniques which may be used may include, but are not limited to, one or more of the following (or combinations thereof): random number generators, SHA-1 (Secured Hashing Algorithm), MD2, MD5, DES (Digital Encryption Standard), 3DES (Triple DES), RC4 (Rivest Cipher), ARC4 (related to RC4), TKIP (Temporal Key Integrity Protocol, uses RC4), AES (Advanced Encryption Standard), RSA, DSA, DH, NTRU, and ECC (elliptic curve cryptography), PKA (Private Key Authentication), Device-Unique Secret Key and other cryptographic key data, SSL, etc. Other security features contemplated may include use of well-known hardware-based and/or software-based security components, and/or any other known or yet to be devised security and/or hardware and encryption/decryption processes implemented in hardware and/or software.

It will be appreciated that the Transaction Analytics System of FIG. 1A is but one example from a wide range of Transaction Analytics System embodiments which may be implemented. Other embodiments of the Transaction Analytics System (not shown) may include additional, fewer and/or different components/features that those illustrated in the exemplary Transaction Analytics System embodiment of FIG. 1A. Generally, the various techniques for implementing a Transaction Analytics System or other similar system described herein may be implemented in software, hardware and/or hardware+software. For example, they can be implemented in an operating system kernel, in a separate user process, in a library package bound into network applications, on a specially constructed machine, or on a network interface card. In a specific embodiment, various aspects described herein may be implemented in software such as an operating system or in an application running on an operating system.

Software, hardware and/or software+hardware hybrid embodiments of the Transaction Analytics System techniques described herein may be implemented on a general-purpose programmable machine selectively activated or reconfigured by a computer program stored in memory. Such programmable machine may include, for example, mobile or handheld computing systems, PDA, smart phones, notebook computers, tablets, netbooks, desktop computing systems, server systems, cloud computing systems, network devices, and the like.

Continuing with FIG. 1B, an exemplary computing device according to one embodiment of the present disclosure is illustrated in front perspective view. Computing device 10, which can be a laptop computer, can be particularly adapted to facilitate various functions for the transaction analysis and asset recovery systems provided herein. It will be readily appreciated that computing device 10 can be provided in numerous other configurations and formats while still being able to provide the disclosed transaction analysis activities, such that the provided laptop example is for illustrative purposes only. For example, computing device 10 could also be a desktop computer, tablet computer, smart phone, personal digital assistant, or the like.

In general, computing device 10 can include an upper portion 11 and a lower portion 12. Upper portion 11 can include a display component 13 having a display region thereupon, while lower portion 12 can include various input devices, such as a keyboard 14 and touchpad 15. Lower portion 12 may also include a processor (not shown) therewithin, which can be adapted to generate or process data for display to a user of a transaction analysis and asset recovery system. Such a processor can be coupled to the display component 13 and the input devices 14, 15, as well as other components of the computing device. Such other computing device components or items not shown may also be included, as will be readily appreciated, with such items including, for example, speakers, memories, busses, input ports, disk drives, power supplies, wireless interfaces, and the like.

FIG. 1C illustrates in front perspective view an alternative exemplary computing device according to one embodiment of the present disclosure. Smart phone 20 can similarly be used to provide or facilitate inputting data, reviewing analysis results, or other similar activity for a transaction analysis and asset recovery system. As in the foregoing computing device 10, smart phone 20 can include at least a processor, display component having a display region, and one or more input devices, such as a touchscreen, button(s) and/or a keypad. In various embodiments, a “TransactionAnalytics” program or the like can be provided as an application or “app” on an app store that can be accessed from smart phone 20. Such an app can be downloaded and then used on the phone 20. Such an app can be specialized for one form of contract or transaction data input, one form of data analysis results review, or can be a universal app configured to provide many functionalities that are available for the provider or a given user of an overall transaction analysis and asset recovery system.

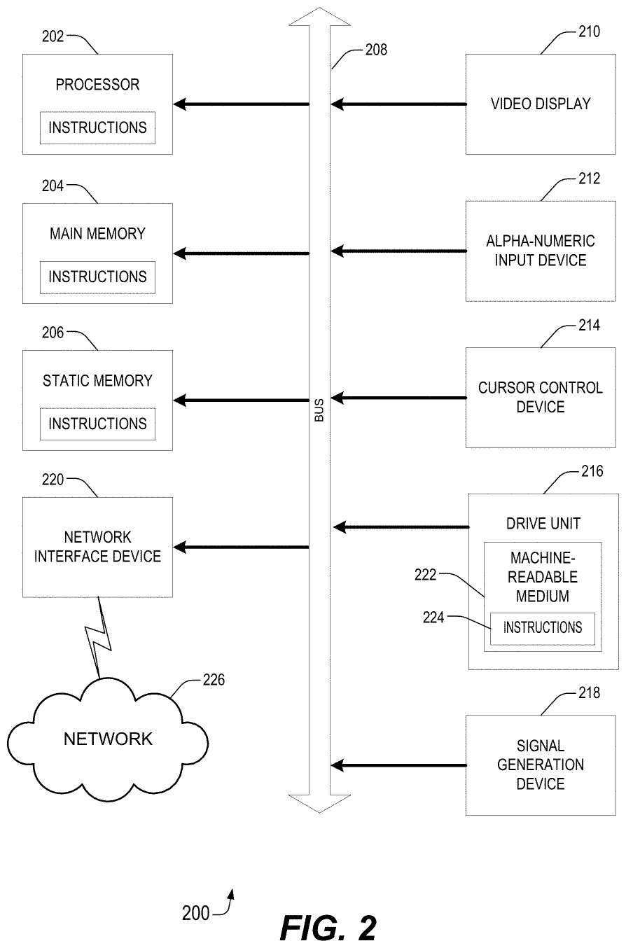

Turning next to FIG. 2, a diagrammatic representation of an exemplary client or end user computing device or system is provided. Computing device or system 200 can be identical or similar to any of the foregoing computer devices 10, 20, as well as any other suitable computing device or system adapted for providing the disclosed Transaction Analytics Systems and components thereof.

Computing device or system 200 may contain a set of instructions for causing itself or another networked machine to perform any one or more of the methodologies discussed herein. As such, computing device or system 200 may operate as a standalone device or machine, or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

Exemplary computer device or system 200 includes a processor 202 (e.g., a central processing unit (CPU), a graphics processing unit (GPU) or both), a main memory 204 and a static memory 206, which communicate with each other via a bus 208. The computer device or system 200 may further include a video display unit 210 (e.g., a liquid crystal display (LCD) or a cathode ray tube (CRT)), and also an alphanumeric input device 212 (e.g., a keyboard), a user interface (UI) navigation device 214 (e.g., a mouse), a disk drive unit 216, a signal generation device 218 (e.g., a speaker) and a network interface device 220. The disk drive unit 216 includes a machine-readable medium 222 on which is stored one or more sets of instructions and data structures (e.g., software 224) embodying or utilized by any one or more of the methodologies or functions described herein. The software 224 may also reside, completely or at least partially, within the main memory 204 and/or within the processor 202 during execution thereof by the computer device or system 200, wherein the main memory 204 and/or the processor 202 may also be constituting machine-readable media.

The software 224 may further be transmitted or received over a network 226 via the network interface device 220 utilizing any one of a number of well-known transfer protocols (e. g., HTTP). While the machine-readable medium 222 is shown in an exemplary embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention, or that is capable of storing, encoding or carrying data structures utilized by or associated with such a set of instructions. The term “machine-readable medium” shall accordingly be taken to include, but not be limited to, solid-state memories, optical and magnetic media, and carrier wave signals.

According to various embodiments, computing device or system 200 may include a variety of components, modules and/or systems for providing various types of functionality. For example, in at least one embodiment, device or system 200 may include a web browser application which is operable to process, execute, and/or support the use of scripts (e.g., JavaScript, AJAX, etc.), Plug-ins, executable code, virtual machines, HTML5 vector-based web animation (e.g., Adobe Flash), and the like. In at least one embodiment, the web browser application may be configured or designed to instantiate components and/or objects at the device or system 200 in response to processing scripts, instructions, and/or other information received from a remote server such as a web server. Examples of such components and/or objects may include, but are not limited to, UI components, database components, processing components, and other components that may facilitate and/or enable device or system 200 to perform and/or initiate various types of operations, activities, functions such as those described herein with respect to providing a transaction analysis and asset recovery system.

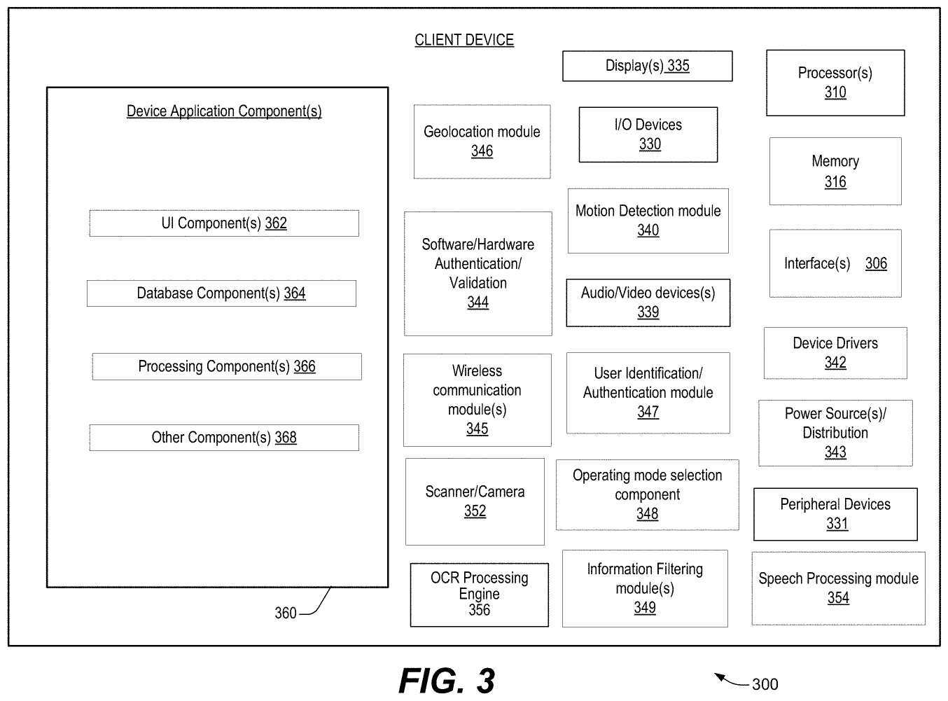

Continuing with FIG. 3, a block diagram of an exemplary client system for a mobile device is provided. In at least one embodiment, the mobile device client system 300 may include a Transaction Analytics System Mobile Device App Component, which has been configured or designed to provide functionality for enabling or implementing at least a portion of the various Transaction Analytics System software modules or components at the mobile device client system. Such a mobile device app component can be provided for download by a service provider, such as an app store for smart phone devices. Various aspects, features, and/or functionalities of client system 300 may be performed, implemented, and/or initiated by one or more of the following types of systems, components, systems, devices, procedures, processes, and the like. Such items can include, for example: Processor(s) 310, Device Drivers 342, Memory 316, Interface(s) 306, Power Source(s)/Distribution 343, Geolocation module 346, Display(s) 335, I/O Devices 330, Audio/Video devices(s) 339, Peripheral Devices 331, Motion Detection module 340, User Identification/Authentication module 347, Client App Component(s) 360, Other Component(s) 368, UI Component(s) 362, Database Component(s) 364, Processing Component(s) 366, Software/Hardware Authentication/Validation 344, Wireless communication module(s) 345, Information Filtering module(s) 349, Operating mode selection component 348, Speech Processing module 354, Scanner/Camera 352, and Optical Character Recognition (“OCR”) Processing Engine 356, among other possible components.

As illustrated in the example of FIG. 3, mobile device 300 may include a variety of components, modules and/or systems for providing various functionality. For example, Mobile Device 300 may include Mobile Device Application components (e.g., 360), which, for example, may include, but are not limited to, one or more of the following (or combinations thereof): UI Components 362, Database Components 364, Processing Components 366, and Other Components 368 which, for example, may include components for facilitating and/or enabling the mobile device to perform and/or initiate various types of operations, activities, functions such as those described herein.

In at least one embodiment, a given instance of the Mobile Device Application component(s) may access and/or utilize information from one or more associated databases. In at least one embodiment, at least a portion of the database information may be accessed via communication with one or more local and/or remote memory devices. Examples of different types of data which may be accessed by the Mobile Device Application component(s) may include, but are not limited to, one or more different types of data, metadata, and/or other information described and/or referenced herein.

According to different embodiments, Mobile Device 300 may further include, but is not limited to, different types of components, modules and/or systems (or combinations thereof) such as, for example, one or more of the following.
At least one processor 310. In at least one embodiment, the processor(s) 310 may include one or more commonly known CPUs that are deployed in many current consumer electronic devices, such as, for example, CPUs or processors from the Motorola or Intel family of microprocessors, etc. In an alternative embodiment, at least one processor may be specially designed hardware for controlling the operations of the client system. In a specific embodiment, a memory (such as non-volatile RAM and/or ROM) also forms part of CPU. When acting under the control of appropriate software or firmware, the CPU may be responsible for implementing specific functions associated with the functions of a desired network device. The CPU preferably accomplishes all these functions under the control of software including an operating system, and any appropriate applications software.
Memory 316, which, for example, may include volatile memory (e.g., RAM), non-volatile memory (e.g., disk memory, FLASH memory, EPROMs, etc.), unalterable memory, and/or other types of memory. In at least one implementation, the memory 316 may include functionality similar to at least a portion of functionality implemented by one or more commonly known memory devices such as those described herein and/or generally known to one having ordinary skill in the art. According to different embodiments, one or more memories or memory modules (e.g., memory blocks) may be configured or designed to store data, program instructions for the functional operations of the client system and/or other information relating to the functionality of the various Transaction Analytics System components described herein. The program instructions may control the operation of an operating system and/or one or more applications, for example. The memory or memories may also be configured to store data structures, metadata, timecode synchronization information, audio/visual media content, asset file information, keyword taxonomy information, advertisement information, and/or information/data relating to other features/functions described herein. Because such information and program instructions may be employed to implement at least a portion of the Transaction Analytics System components described herein, various aspects described herein may be implemented using machine readable media that include program instructions, state information, etc. Examples of machine-readable media include, but are not limited to, magnetic media such as hard disks, floppy disks, and magnetic tape; optical media such as CD-ROM disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.
Interface(s) 306 which, for example, may include wired interfaces and/or wireless interfaces. In at least one implementation, the interface(s) 306 may include functionality similar to at least a portion of functionality implemented by one or more computer system interfaces such as those described herein and/or generally known to one having ordinary skill in the art. For example, in at least one implementation, the wireless communication interface(s) may be configured or designed to communicate with selected Transaction Analytics System components, computer systems, remote servers, other wireless devices (e.g., PDAs, cell phones, player tracking transponders, etc.), etc. Such wireless communication may be implemented using one or more wireless interfaces/protocols such as, for example, 802.11 (WiFi), 802.15 (including Bluetooth™), 802.16 (WiMax), 802.22, Cellular standards such as CDMA, CDMA2000, WCDMA, Radio Frequency (e.g., RFID), Infrared, Near Field Magnetics, etc.
Device driver(s) 342. In at least one implementation, the device driver(s) 342 may include functionality similar to at least a portion of functionality implemented by one or more computer system driver devices such as those described herein and/or generally known to one having ordinary skill in the art.
At least one power source (and/or power distribution source) 343. In at least one implementation, the power source may include at least one mobile power source (e.g., battery) for allowing the client system to operate in a wireless and/or mobile environment. For example, in one implementation, the power source 343 may be implemented using a rechargeable, thin-film type battery. Further, in embodiments where it is desirable for the device to be flexible, the power source 343 may be designed to be flexible.
Geolocation module 346 which, for example, may be configured or designed to acquire geolocation information from remote sources and use the acquired geolocation information to determine information relating to a relative and/or absolute position of the client system.
Motion detection component 340 for detecting motion or movement of the client system and/or for detecting motion, movement, gestures and/or other input data from user. In at least one embodiment, the motion detection component 340 may include one or more motion detection sensors such as, for example, MEMS (Micro Electro Mechanical System) accelerometers, that can detect the acceleration and/or other movements of the client system as it is moved by a user.
User Identification/Authentication module 347. In one implementation, the User Identification module may be adapted to determine and/or authenticate the identity of the current user or owner of the client system. For example, in one embodiment, the current user may be required to perform a log in process at the client system in order to access one or more features. Alternatively, the client system may be adapted to automatically determine the identity of the current user based upon one or more external signals such as, for example, an RFID tag or badge worn by the current user that provides a wireless signal to the client system for determining the identity of the current user. In at least one implementation, various security features may be incorporated into the client system to prevent unauthorized users from accessing confidential or sensitive information.
One or more display(s) 335. According to various embodiments, such display(s) may be implemented using, for example, LCD display technology, OLED display technology, and/or other types of conventional display technology.
One or more user I/O Device(s) 330 such as, for example, keys, buttons, scroll wheels, cursors, touchscreen sensors, audio command interfaces, magnetic strip reader, optical scanner, etc.
Audio/Video device(s) 339 such as, for example, components for displaying audio/visual media which, for example, may include cameras, speakers, microphones, media presentation components, wireless transmitter/receiver devices for enabling wireless audio and/or visual communication between the client system 300 and remote devices (e.g., radios, telephones, computer systems, etc.). For example, in one implementation, the audio system may include componentry for enabling the client system to function as a cell phone or two-way radio device.
Other types of peripheral devices 331 which may be useful to the users of various client systems, such as, for example: PDA functionality; memory card reader(s); fingerprint reader(s); image projection device(s); social networking peripheral component(s); etc.
Information filtering module(s) 349 which, for example, may be adapted to automatically and dynamically generate, using one or more filter parameters, filtered information to be displayed on one or more displays of the mobile device. In one implementation, such filter parameters may be customizable by the player or user of the device. In some embodiments, information filtering module(s) 349 may also be adapted to display, in real-time, filtered information to the user based upon a variety of criteria such as, for example, geolocation information, contextual activity information, and/or other types of filtering criteria described and/or referenced herein.
Wireless communication module(s) 345. In one implementation, the wireless communication module 345 may be configured or designed to communicate with external devices using one or more wireless interfaces/protocols such as, for example, 802.11 (WiFi), 802.15 (including Bluetooth™), 802.16 (WiMax), 802.22, Cellular standards such as CDMA, CDMA2000, WCDMA, Radio Frequency (e.g., RFID), Infrared, Near Field Magnetics, etc.
Software/Hardware Authentication/validation components 344 which, for example, may be used for authenticating and/or validating local hardware and/or software components, hardware/software components residing at a remote device, Transaction Analytics System information, user information and/or identity, etc.
Operating mode selection component 348 which, for example, may be operable to automatically select an appropriate mode of operation based on various parameters and/or upon detection of specific events or conditions such as, for example: the mobile device's current location; identity of current user; user input; system override (e.g., emergency condition detected); proximity to other devices belonging to same group or association; proximity to specific objects, regions, zones, etc. Additionally, the mobile device may be operable to automatically update or switch its current operating mode to the selected mode of operation. The mobile device may also be adapted to automatically modify accessibility of user-accessible features and/or information in response to the updating of its current mode of operation.
Scanner/Camera Component(s) (e.g., 352) which may be configured or designed for use in scanning identifiers and/or other content from other devices and/or objects such as for example: mobile device displays, computer displays, static displays (e.g., printed on tangible mediums), etc.
OCR Processing Engine (e.g., 356) which, for example, may be operable to perform image processing and optical character recognition of images such as those captured by a mobile device camera, for example.
Speech Processing module (e.g., 354) which, for example, may be operable to perform speech recognition, and may be operable to perform speech-to-text conversion.

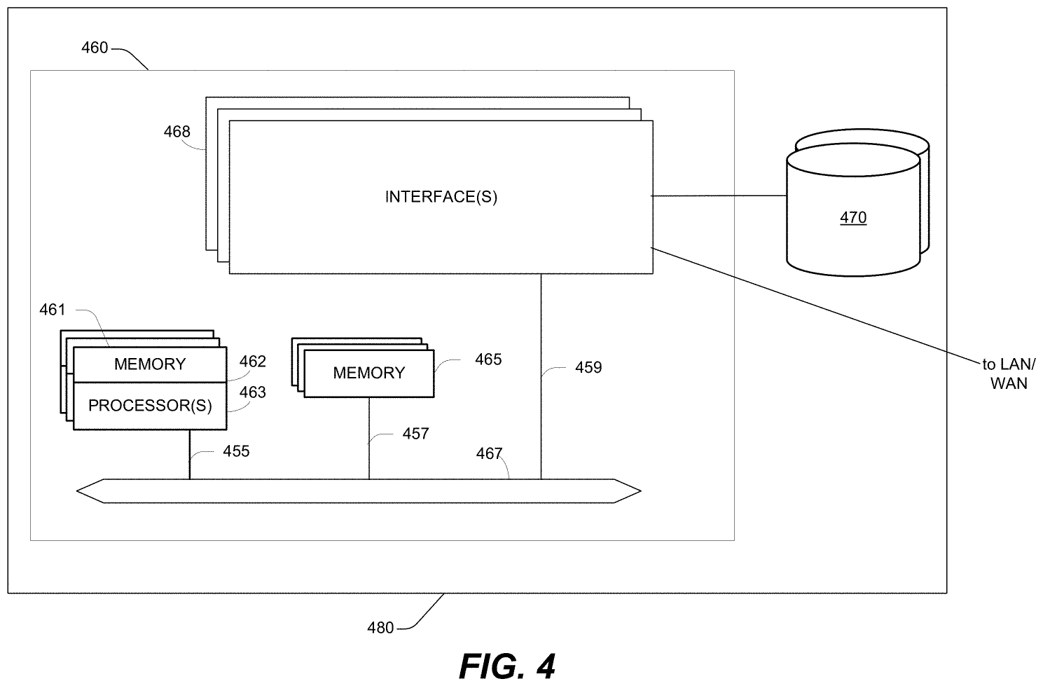

FIG. 4 illustrates in block diagram format an exemplary server system 480, which may be used for implementing various aspects/features described herein. In at least one embodiment, the server system 480 includes at least one network device 460, and at least one storage device 470 (such as, for example, a direct attached storage device). In one embodiment, server system 480 may be suitable for implementing at least some of the Transaction Analytics System techniques described herein.

According to one embodiment, network device 460 may include a master central processing unit (CPU) 462, interfaces 468, and a bus 467 (e.g., a PCI bus). When acting under the control of appropriate software or firmware, the CPU 462 may be responsible for implementing specific functions associated with the functions of a desired network device. For example, when configured as a server, the CPU 462 may be responsible for analyzing packets; encapsulating packets; forwarding packets to appropriate network devices; instantiating various types of virtual machines, virtual interfaces, virtual storage volumes, virtual appliances; etc. The CPU 462 preferably accomplishes at least a portion of these functions under the control of software including an operating system (e.g. Linux), and any appropriate system software (such as, for example, AppLogic™ software).

CPU 462 may include one or more processors 463 such as, for example, one or more processors from the AMD, Motorola, Intel and/or MIPS families of microprocessors. In an alternative embodiment, processor 463 may be specially designed hardware for controlling the operations of server system 480. In a specific embodiment, a memory 461 (such as non-volatile RAM and/or ROM) also forms part of CPU 462. However, there may be many different ways in which memory could be coupled to the system. Memory block 461 may be used for a variety of purposes such as, for example, caching and/or storing data, programming instructions, etc.

The interfaces 468 may be typically provided as interface cards (sometimes referred to as “line cards”). Alternatively, one or more of the interfaces 468 may be provided as on-board interface controllers built into the system motherboard. Generally, they control the sending and receiving of data packets over the network and sometimes support other peripherals used with the server system 480. Among the interfaces that may be provided may be FC interfaces, Ethernet interfaces, frame relay interfaces, cable interfaces, DSL interfaces, token ring interfaces, Infiniband interfaces, and the like. In addition, various very high-speed interfaces may be provided, such as fast Ethernet interfaces, Gigabit Ethernet interfaces, ATM interfaces, HSSI interfaces, POS interfaces, FDDI interfaces, ASI interfaces, DHEI interfaces and the like. Other interfaces may include one or more wireless interfaces such as, for example, 802.11 (WiFi) interfaces, 802.15 interfaces (including Bluetooth™), 802.16 (WiMax) interfaces, 802.22 interfaces, Cellular standards such as CDMA interfaces, CDMA2000 interfaces, WCDMA interfaces, TDMA interfaces, Cellular 3G interfaces, and the like.

Generally, one or more interfaces may include ports appropriate for communication with the appropriate media. In some cases, they may also include an independent processor and, in some instances, volatile RAM. The independent processors may control such communications intensive tasks as packet switching, media control and management. By providing separate processors for the communications intensive tasks, these interfaces allow the master microprocessor 462 to efficiently perform routing computations, network diagnostics, security functions, and the like.

In at least one embodiment, some interfaces may be configured or designed to allow the server system 480 to communicate with other network devices associated with various local area network (LANs) and/or wide area networks (WANs). Other interfaces may be configured or designed to allow network device 460 to communicate with one or more direct attached storage device(s) 470.

Although the system shown in FIG. 4 illustrates one specific network device described herein, it is by no means the only network device architecture on which one or more embodiments can be implemented. For example, an architecture having a single processor that handles communications as well as routing computations, etc. may be used. Further, other types of interfaces and media could also be used with the network device.

Regardless of network device configuration, a network may employ one or more memories or memory modules (such as, for example, memory block 465, which, for example, may include random access memory (RAM)) configured to store data, program instructions for the general-purpose network operations and/or other information relating to the functionality of the various Transaction Analytics System techniques described herein. The program instructions may control the operation of an operating system and/or one or more applications, for example. The memory or memories may also be configured to store data structures, and/or other specific non-program information described herein.

Because such information and program instructions may be employed to implement the systems/methods described herein, one or more embodiments relates to machine readable media that include program instructions, state information, etc. for performing various operations described herein. Examples of machine-readable storage media include, but are not limited to, magnetic media such as hard disks, floppy disks, and magnetic tape; optical media such as CD-ROM disks; magneto-optical media such as floptical disks; and hardware devices that may be specially configured to store and perform program instructions, such as read-only memory devices (ROM) and random access memory (RAM). Some embodiments may also be embodied in transmission media such as, for example, a carrier wave travelling over an appropriate medium such as airwaves, optical lines, electric lines, etc. Examples of program instructions include both machine code, such as produced by a compiler, and files containing higher level code that may be executed by the computer using an interpreter.

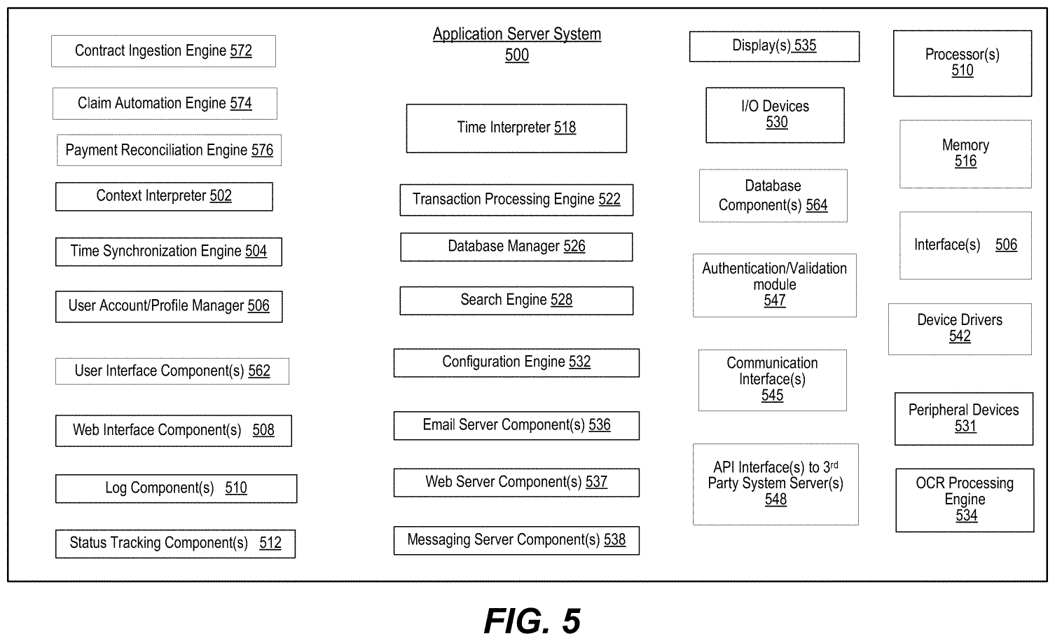

FIG. 5 provides a block diagram of an application server system. Application server system 500 may be operable to perform and/or implement various types of functions, operations, actions, and/or other features associated with providing a Transaction Analytics System. In at least one embodiment, application server system 500 may include a plurality of components operable to perform and/or implement various types of functions, operations, actions, and/or other features such as, for example:
Contract Ingestion Engine (e.g., 572) which, for example, may be operable to automatically intake and process contract data, as well as populate a Contract Rules Database with the processed contract data, as set forth below.
Claim Automation Engine (e.g., 574) which, for example, may be operable to automatically intake and process claims data, as well as populate a Claims Database with the processed claims data, as set forth below.
Payment Reconciliation Engine (e.g., 576) which, for example, may be operable to perform analytics on the processed contract data and processed claims data to result in a variety of different outputs, such as recovering lost assets, as set forth below.
Context Interpreter (e.g., 502) which, for example, may be operable to automatically and/or dynamically analyze contextual criteria relating to a detected set of event(s) and/or condition(s), and automatically determine or identify one or more contextually appropriate response(s) based on the contextual interpretation of the detected event(s)/condition(s). According to different embodiments, examples of contextual criteria which may be analyzed may include, but are not limited to, one or more of the following (or combinations thereof):
   location-based criteria (e.g., geolocation of client device, geolocation of agent device, etc.)
   time-based criteria
   identity of user(s)
   user profile information
   transaction history information
   recent user activities
   proximate business-related criteria
Time Synchronization Engine (e.g., 504) which, for example, may be operable to manages universal time synchronization (e.g., via NTP and/or GPS)
Search Engine (e.g., 528) which, for example, may be operable to search for transactions, logs, items, accounts, options in the Transaction Analytics System databases
Configuration Engine (e.g., 532) which, for example, may be operable to determine and handle configuration of various customized configuration parameters for one or more devices, component(s), system(s), process(es), and so forth.
Time Interpreter (e.g., 518) which, for example, may be operable to automatically and/or dynamically modify or change identifier activation and expiration time(s) based on various criteria such as, for example, time, location, transaction status, and the like.
Authentication/Validation Component(s) (e.g., 547) (password, software/hardware info, SSL certificates) which, for example, may be operable to perform various types of authentication/validation tasks such as, for example, one or more of the following (or combinations thereof):
   verifying/authenticating devices,
   verifying passwords, passcodes, SSL certificates, biometric identification information, and/or other types of security-related information
   verify/validate activation and/or expiration times
In one implementation, the Authentication/Validation Component(s) may be adapted to determine and/or authenticate the identity of the current user or owner of the mobile client system. For example, in one embodiment, the current user may be required to perform a log in process at the mobile client system in order to access one or more features. In some embodiments, the mobile client system may include biometric security components which may be operable to validate and/or authenticate the identity of a user by reading or scanning The user's biometric information (e.g., fingerprints, face, voice, eye/iris, etc.). In at least one implementation, various security features may be incorporated into the mobile client system to prevent unauthorized users from accessing confidential or sensitive information.
Transaction Processing Engine (e.g., 522) which, for example, may be operable to handle various types of transaction processing tasks such as, for example, one or more of the following (or combinations thereof):
   identifying/determining transaction type
   determining which payment gateway(s) to use
   associating databases information to identifiers
OCR Processing Engine (e.g., 534) which, for example, may be operable to perform image processing and optical character recognition of images such as those captured by a mobile device camera, for example.
Database Components(s) (eg, 564) which, for example, may include one or more local databases and/or may include components for accessing one or more remote databases. In at least one embodiment, the database components(s) 564 may be configured or designed to provide access to the Contract Rules Database, Claims Database, and/or Reconciliation Database.
Database Manager (e.g., 526) which, for example, may be operable to handle various types of tasks relating to database updating, database management, database access, etc. In at least one embodiment, the Database Manager may be operable to manage Transaction Analytics System Application databases.
Log Component(s) (e.g., 510) which, for example, may be operable to generate and manage transactions history logs, system errors, connections from APIs, and the like.
Status Tracking Component(s) (e.g., 512) which, for example, may be operable to automatically and/or dynamically determine, assign, and/or report updated transaction status information based, for example, on the state of the transaction. In at least one embodiment, the status of a given transaction may be reported as one or more of the following (or combinations thereof): Completed, Incomplete, Pending, Invalid, Error, Declined, Accepted, Other.
Gateway Component(s) (e.g., 514) which, for example, may be operable to facilitate and manage communications and transactions with external Payment Gateways.
Web Interface Component(s) (e.g., 508) which, for example, may be operable to facilitate and manage communications and transactions with Transaction Analytics System web portal(s).
API Interface(s) to Application Server System(s) (e.g., 546) which, for example, may be operable to facilitate and manage communications and transactions with API Interface(s) to Application Server System(s)
API Interface(s) to 3rd Party Server System(s) (e.g., 548) which, for example, may be operable to facilitate and manage communications and transactions with API Interface(s) to 3rd Party Server System(s)
OCR Processing Engine (e.g., 534) which, for example, may be operable to perform image processing and optical character recognition of images such as those captured by a mobile device camera, for example.
At least one processor 510. In at least one embodiment, the processor(s) 510 may include one or more commonly known CPUs which are deployed in many of today's consumer electronic devices, such as, for example, CPUs or processors from the Motorola or Intel family of microprocessors, etc. In an alternative embodiment, at least one processor may be specially designed hardware for controlling the operations of the mobile client system. In a specific embodiment, a memory (such as non-volatile RAM and/or ROM) also forms part of CPU. When acting under the control of appropriate software or firmware, the CPU may be responsible for implementing specific functions associated with the functions of a desired network device. The CPU preferably accomplishes all these functions under the control of software including an operating system, and any appropriate applications software.
Memory 516, which, for example, may include volatile memory (e.g., RAM), non-volatile memory (e.g., disk memory, FLASH memory, EPROMs, etc.), unalterable memory, and/or other types of memory. In at least one implementation, the memory 516 may include functionality similar to at least a portion of functionality implemented by one or more commonly known memory devices such as those described herein and/or generally known to one having ordinary skill in the art. According to different embodiments, one or more memories or memory modules (e.g., memory blocks) may be configured or designed to store data, program instructions for the functional operations of the mobile client system and/or other information relating to the functionality of the various Mobile Transaction techniques described herein. The program instructions may control the operation of an operating system and/or one or more applications, for example. The memory or memories may also be configured to store data structures, metadata, identifier information/images, and/or information/data relating to other features/functions described herein. Because such information and program instructions may be employed to implement at least a portion of the Transaction Analytics System techniques described herein, various aspects described herein may be implemented using machine readable media that include program instructions, state information, etc. Examples of machine-readable media include, but are not limited to, magnetic media such as hard disks, floppy disks, and magnetic tape; optical media such as CD-ROM disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.
Interface(s) 506 which, for example, may include wired interfaces and/or wireless interfaces. In at least one implementation, the interface(s) 506 may include functionality similar to at least a portion of functionality implemented by one or more computer system interfaces such as those described herein and/or generally known to one having ordinary skill in the art.
Device driver(s) 542. In at least one implementation, the device driver(s) 542 may include functionality similar to at least a portion of functionality implemented by one or more computer system driver devices such as those described herein and/or generally known to one having ordinary skill in the art.
One or more display(s) 535. According to various embodiments, such display(s) may be implemented using, for example, LCD display technology, OLED display technology, and/or other types of conventional display technology.
Email Server Component(s) 536, which, for example, may be configured or designed to provide various functions and operations relating to email activities and communications.
Web Server Component(s) 537, which, for example, may be configured or designed to provide various functions and operations relating to web server activities and communications.
Messaging Server Component(s) 538, which, for example, may be configured or designed to provide various functions and operations relating to text messaging and/or other social network messaging activities and/or communications.

Again, while the exemplary physical items and arrangements of computing systems and components thereof provided herein may be one way of implementing the functions of the disclosed transaction analysis and asset recovery systems and methods, many other types of computing systems and alternative components may also be used.

### Overall Transaction Analysis and Asset Recovery System

As noted above, the disclosed transaction analysis and asset recovery systems may be used for a variety of different industries and relational arrangements. For purposes of illustration and discussion moving forward, specific arrangements involving healthcare providers and insurance company payors will be used. This can involve terminology specific to this industry that may not reflect a more generalized terminology for other industries. For example, the specific term “claims” may be used rather than the more generic term “transactions” to refer to actual service instances or transactions. Other specific terms may also be used in the following discussions, and one of skill in the art will readily appreciate that such terms may be genericized or even altered to other specific terms to reflect a different industry or relational arrangement using the disclosed systems.

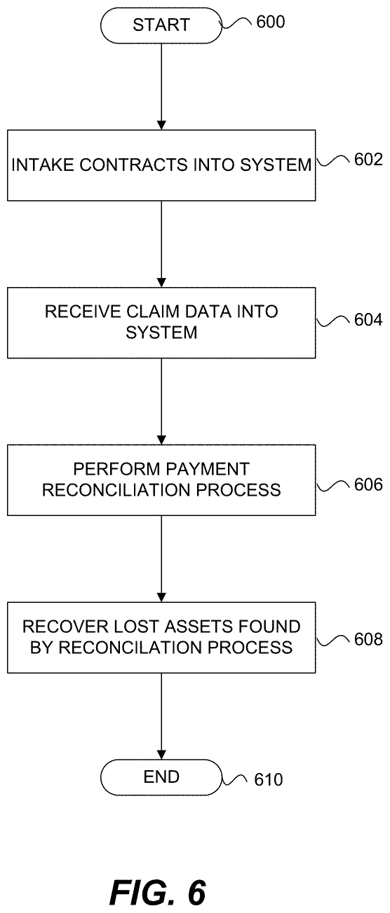

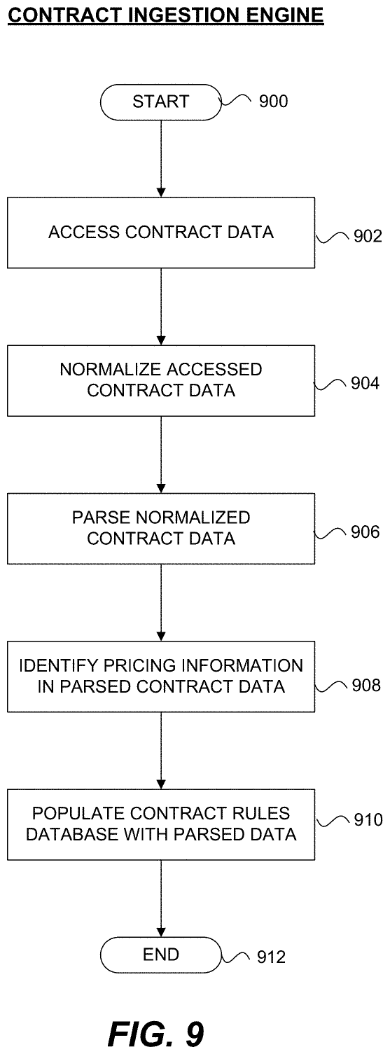

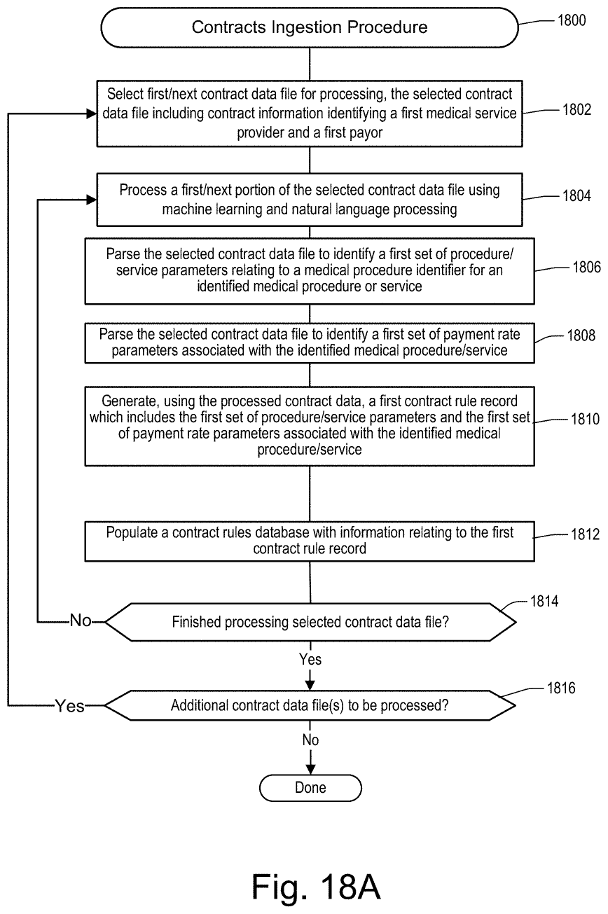

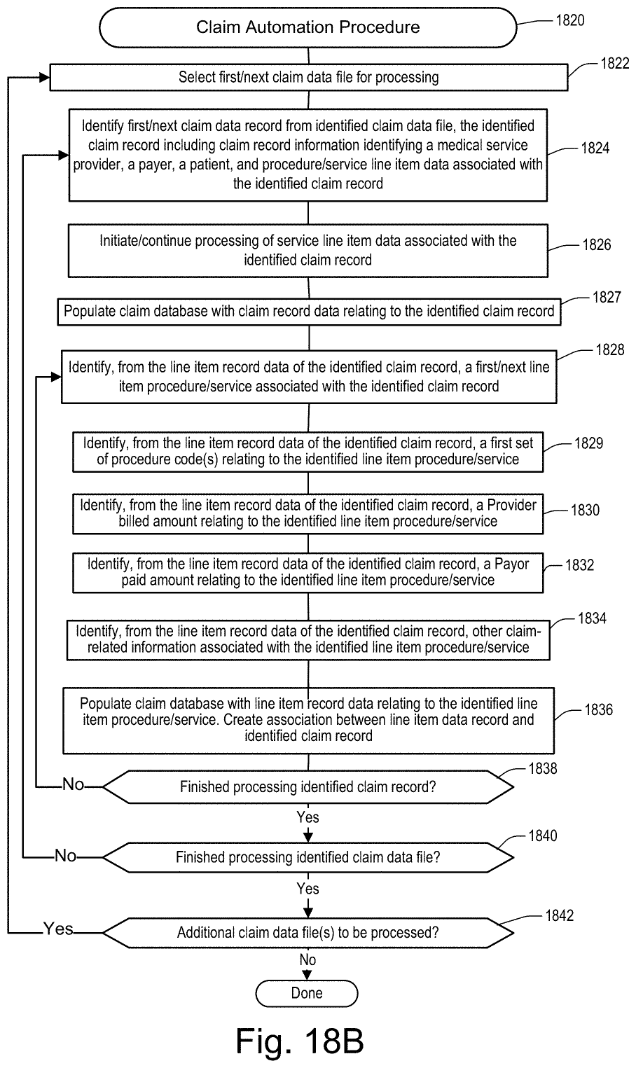

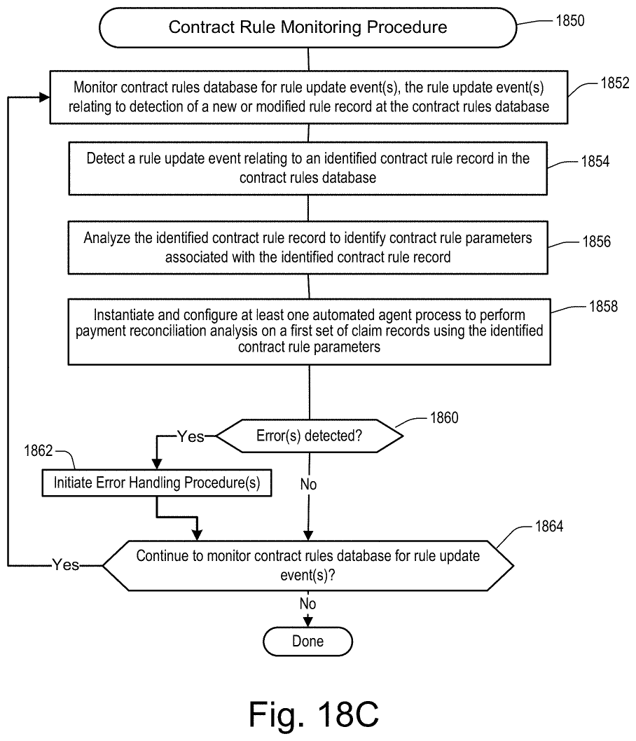

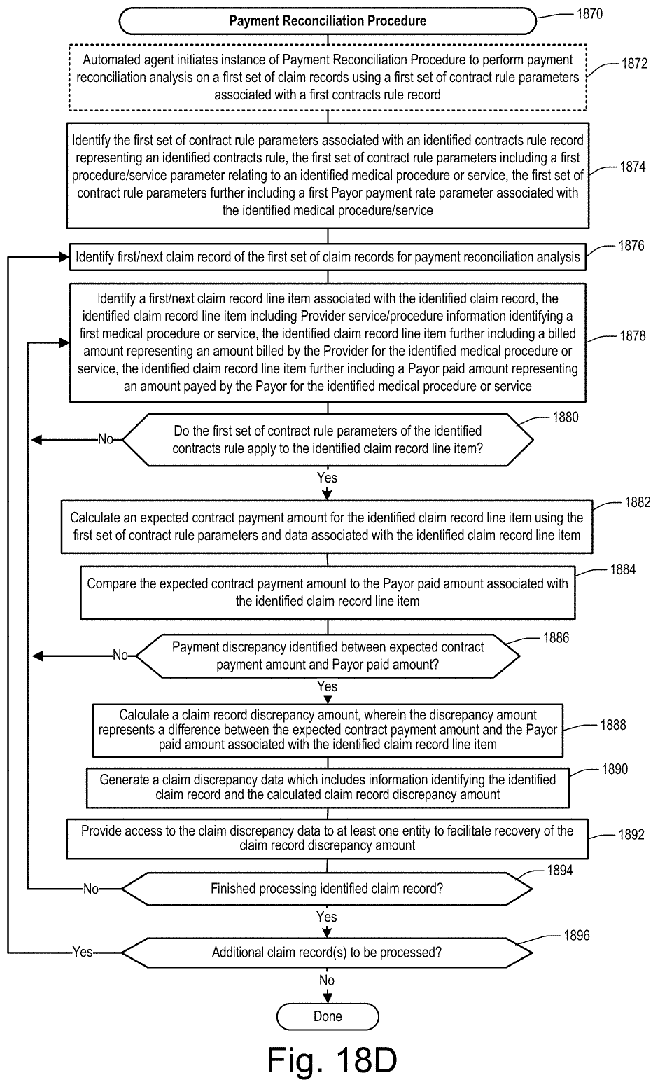

Transitioning now to FIG. 6, a flowchart of an exemplary overall method of analyzing transactions using a computing system is provided. It will be understood that the method shown here is a broad overall process, and that each step may be broken down into additional steps and details. After a start event 600, a first process step 602 can involve importing contract data into the computing system. This can include, for example, contracts between healthcare providers and health insurance companies as to how healthcare services and goods will be paid. Each healthcare provider may have various contracts with different insurance companies, and each insurance company may have various contracts with different healthcare providers. Contracts that are input into the system can be in a variety of different formats, and data from the contracts can be put into a database, such as a contract rules database. Multiple contract rules databases may be used. Further details regarding the intake of contracts are provided throughout this disclosure, such as at FIGS. 9 and 18A.

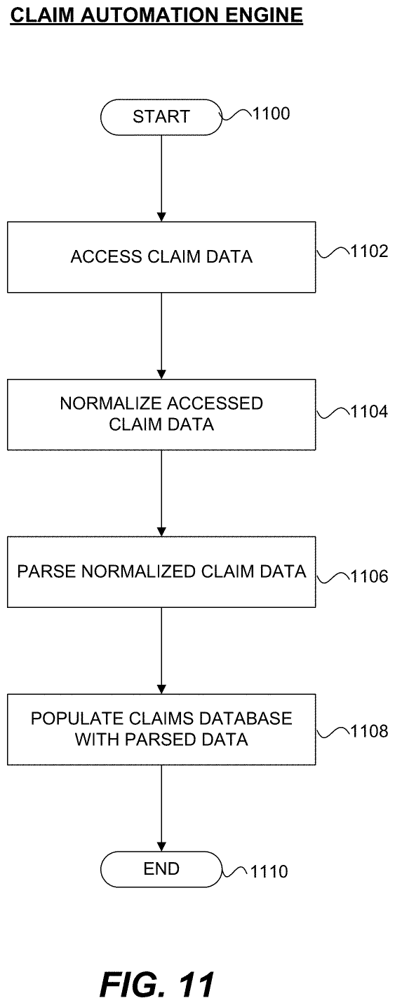

At the next process step 604, claim data can be received into the computing system. This can include, for example, various claims made for health services and goods provided to patients by the healthcare organizations. Each patient claim may correspond to a particular insurance company for that patient, and each claim may include multiple claim items. Claims received into the system can be in different formats, although many claims may be in a standardized electronic data interchange (“EDI”) format. Data from the claims can be placed into a database, such as a claim instances database, and multiple claim instances databases may be used. Further details regarding receiving claim data into the system are provided throughout this disclosure, such as at FIGS. 11, 12, and 18B.

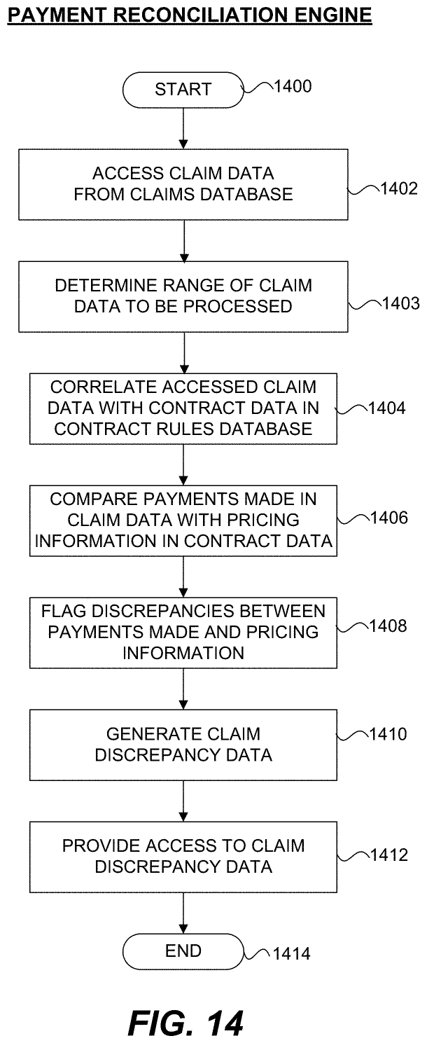

At subsequent process step 606, a payment reconciliation process can be performed. This can include, for example, comparing payments made for claims against payments that should have been made, both as billed and under contract terms for that claim Data regarding payments made and billed for claims can be collected from the claim instances database(s), while data regarding contract terms can be collected from the contract rules database(s). Discrepancies can be flagged for action by the system, which actions can include recovering lost assets based on billing or payment errors, searching the system for similar discrepancies, creating new rules based on flagged discrepancies, applying new rules to current and future billings, and identifying trends, among other possible actions. Further details regarding performing payment reconciliation processes are provided throughout this disclosure, such as at FIGS. 14-17 and 18C-18D.

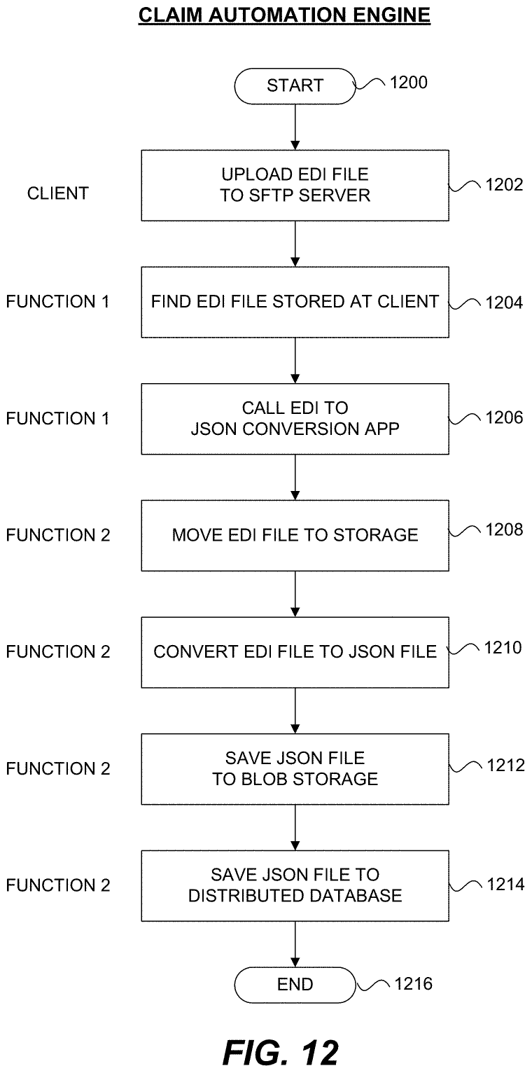

At final process step 608, lost assets found by the payment reconciliation process can be recovered. This can include, for example, rebilling insurance companies in full for various billing or payment errors, negotiating settlements for groups of similar types of billing or payment errors, filing appeals to recover lost or improper payments, and other forms of asset recovery. Further details regarding recovering lost assets found by reconciliation processes are provided throughout this disclosure, such as at FIG. 12. The method can then end at end event 610

For the foregoing flowchart, it will be readily appreciated that the overall method is presented at a high level, that not every method step provided is always necessary, and that further steps not set forth herein may also be included. For example, added steps can involve normalizing and parsing data, among other possible steps. Furthermore, the exact order of steps may be altered as desired, and some steps may be performed simultaneously. For example, steps 602 and 604 can be considered as being performed in parallel and asynchronously in various embodiments. Again, while the provided examples are with respect to transactions between healthcare providers and insurance companies, it will be readily appreciated that the provided methods can also be used with respect to other services and transactions, such as, for example, building construction contracting and subcontracting, labor procurement and delivery, commodities trading, supply chain and logistics, and media advertising, as well as any other alternative industry and relational arrangement with high volume transactions based on contractual obligations, as well as other alternative embodiments.

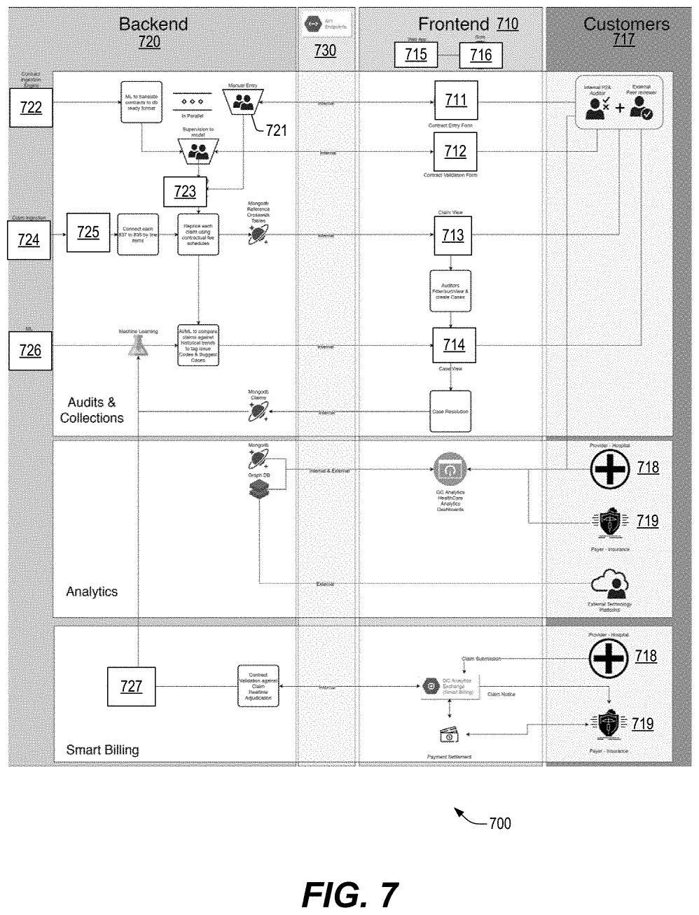

Moving next to FIG. 7, a block diagram of an exemplary overall computing system for analyzing transactions and recovering assets is provided. System 700 can include a system front end 710 having user terminals 711, 712, 713, 714 configured for inputting data and reviewing data analysis results, as well as a system back end 720 having multiple different engines performing functions on the input data. An application programming interface (“API”) layer 730 can facilitate communications between various disparate front end and back end components.

System front end, which can be implemented on web applications 715 and robotic helpers 716, for example, among other possibilities, can include a manual contract entry terminal 711 configured for the manual entry of contract data, an automated contract entry and validation terminal 712 configured for the automated entry and validation of contract data, a claim entry terminal 713 configured for the entry of claim instances, and a case view terminal 714 configured for the review of data and analysis results, among other possible terminals In some embodiments, one or more of terminals 711, 712, 713, 714 can be configured for multiple or all of the foregoing input and output system functions.

System front end 710 can be accessible to various front-end users, which can include the host of the system as well as various clients. Front end users can include internal and external reviewers and auditors 717, healthcare providers 718, and insurance companies 719, among other possible users. Front end users can have the ability to input contract data into the system, such as at terminals 711 and 712, input claim instance data into the system, such as at terminal 713, and review various types of data and data analysis results, such as at terminal 714. In various embodiments, system user profiles types can include, for example:
Host personnel
Hospitals
Doctors
Patients
Insurance Companies
Peer Review—Auditors
Other

System back end 720 can be distributed away from system front end 710 and can be accessible only to the host 721 of the system in some embodiments. System back end 720 can include a Contract Ingestion Engine 722, a Contract Rules Database 723, a Claim Automation Engine 724, a Claim Instances Database 725, a Payment Reconciliation Engine 726, and a Smart Billing Module 727. Various functions and capabilities for each of these system components are set forth in greater detail below.

In various arrangements, the disclosed transaction analysis and asset recovery systems can include system documentation for some or all of the different portions of the system. System documentation may be made available to various parties, such as host operators, auditors, regulators, and the like. Such system documentation can include, for example, but are not limited to:
Data dictionary, design and schema
Functional design elements—dB architecture
Issue code generation/maintenance
Revenue code association
CPT codes
Care Intelligence
Standard Operating Procedures (SOP)
Cloud Infrastructure—Azure I A WS/Google
Collaboration—code and team
Standard integrations
Other

In addition, the disclosed transaction analysis and asset recovery systems can include security components for some or all of the different portions of the system. This can be particularly useful in the exemplary arrangement involving healthcare providers and insurers so that information subject to HIPPA requirements is properly secured, for example. Such system security items can include, for example, but are not limited to:
New VPN protocols to connect to any of the VMs
Integrations for using Azure AD
Shutdown of public facing ports
Data encryption on disk and in database
Corp centralized systems administration
Security reviews for HIPPA compliance
Masking of non-essential subscriber data
Other

### System Back End

The system back end can include various engines for processing and analyzing large amounts of data in multiple databases. Such engines can include a Contract Ingestion Engine, a Claim Automation Engine, and a Payment Reconciliation Engine, among other possible engines and modules. Distributed databases such as a Contract Rules Database and a Claims Database can be used to support these engines, and a Smart Billing Module can be used to support the Payment Reconciliation Engine.

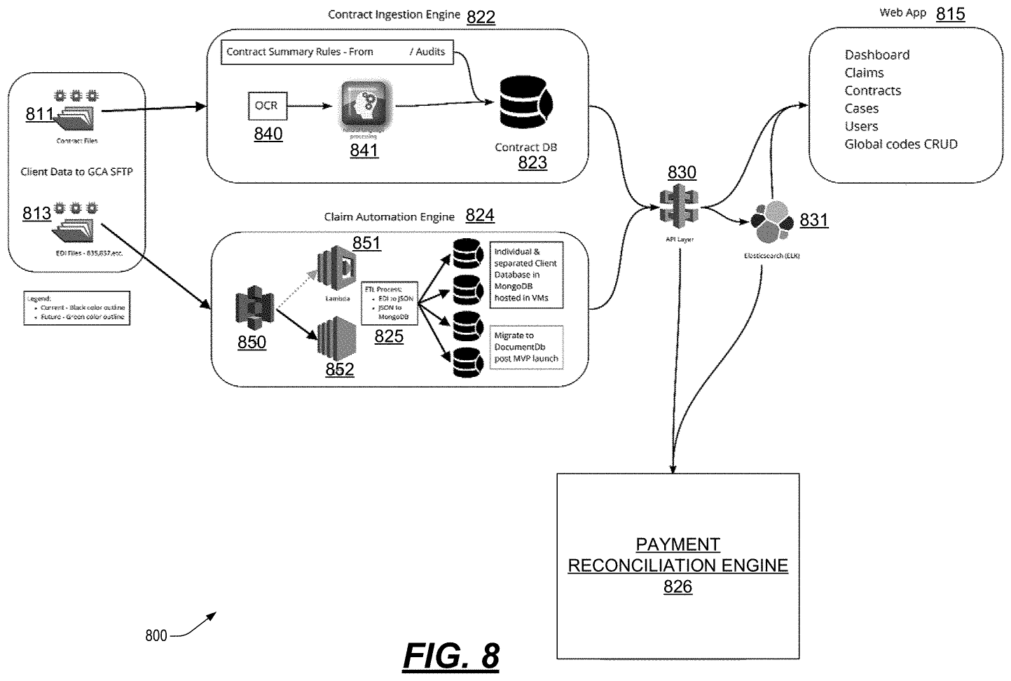

Turning next to FIG. 8, a block diagram of an exemplary back end portion of a computing system for analyzing transactions and recovering assets is illustrated. Transaction analysis and asset recovery system backend 800 can include a Contract Ingestion Engine 822, a Claim Automation Engine 824, and a Payment Reconciliation Engine 826. A Contract Rules Database 823 can be part of the Contract Ingestion Engine 823, while a Claims Database 825 can be part of the Claim Automation Engine 824. In various embodiments, one or both of Contract Rules Database 823 and Claims Database 825 can be distributed and separated databases with individual portions. For example, Claims Database 825 can be separated by client, such that data for only one client resides on any individual portion of the overall distributed and separated Claims Database. In some arrangements, one or both of Contract Rules Database 823 and Claims Database 825 can reside outside of their respective engines, such as that which is shown in FIG. 1A. In any such arrangement, one or both of Contract Rules Database 823 and Claims Database 825 may be accessible to other system components in addition to their respective engines. For example, Payment Reconciliation Engine 826 and its various autonomous agents may have direct access to Contract Rules Database 823, Claims Database 825, or both.

Client data can be in the form of contract files and claim files, among other forms of client data. Contract files can be input into the Contract Ingestion Engine 822 by way of a contract file input portal or terminal 811, while claim files can be input into the Claim Automation Engine 824 by way of a claim file input portal or terminal 813. Contract file data imported into Contract Ingestion Engine 822 can be automatically processed by various engine modules or components, such as an OCR module 840, natural language processing (“NLP”) module 841, and machine learning programs, before then being stored as contract rules in the Contract Rules Database 823.

Claim data can be in the form of EDI files, for example, which can include 837 EDI files for invoices and 835 EDI files for payments in the case of healthcare provider and insurance payor relationship arrangements. Claim file data imported into Claim Automation Engine 824 can be automatically processed by various engine modules or components, such as temporary storage and service function modules 850, 851, 852, which can be microservices on a serverless cloud architecture that convert data from an EDI format to a JavaScript Object Notation (“JSON”) format, among other functions, before then being stored as claim data sets in the Claims Database 825.

Payment Reconciliation Engine 836 and its various agents can have direct access to Contract Ingestion Engine 822, its Contract Rules Database 823, Claim Automation Engine 824, and its Claims Database 825, or access between these engines can be by way of an API layer 830. Further details regarding Payment Reconciliation Engine 836, its agents, and its various functions are provided below. API layer 830 can also exist between the various engines and system front end, which can include a search module 831 and various front-end items, such as a web application 815.

### Example Procedures and Flow Diagrams

FIGS. 9-17 and 18A-18D below illustrate various example embodiments of different automatic transaction analysis and asset recovery procedures and/or procedural flows, as well as example record creation outcomes, which may be used for facilitating activities relating to one or more of the transaction analysis and asset recovery aspects disclosed herein. Again, as shown in the specific examples provided herein for purposes of illustration, these automated procedural flows and outcomes reflect relationships between healthcare providers and insurance company payors, such that specific terminology is used in the examples given. For example, transactions are referred to as claims throughout these examples. It will be readily appreciated that the following examples may be applied to other service provider and payor relationships, and that the specific terminologies, procedural steps, and outcomes provided can be extrapolated to such other arrangements.

According to different embodiments, at least a portion of the various types of functions, operations, actions, outcomes, and/or other features provided by the example transaction analysis and asset recovery procedures provided may be implemented at one or more client systems(s), at one or more server systems (s), and/or combinations thereof. For purposes of discussion, the overall sum of the various procedural flows and possible outcomes described herein shall be termed the “Transaction Analysis Procedure.” In at least one embodiment, the Transaction Analysis Procedure may be operable to perform and/or implement various types of functions, operations, actions, and/or other features such as one or more of those described and/or referenced herein.

In at least one embodiment, the Transaction Analysis Procedure may be operable to utilize and/or generate various different types of data and/or other types of information when performing specific tasks and/or operations. This may include, for example, input data/information and/or output data/information. For example, in at least one embodiment, the Transaction Analysis Procedure may be operable to access, process, and/or otherwise utilize information from one or more different types of sources, such as, for example, one or more local and/or remote memories, devices and/or systems.

Additionally, in at least one embodiment, the Transaction Analysis Procedure may be operable to generate one or more different types of output data/information, which, for example, may be stored in memory of one or more local and/or remote devices and/or systems. Examples of different types of input data/information and/or output data/information which may be accessed and/or utilized by the Transaction Analysis Procedure may include, but are not limited to, one or more of those described and/or referenced herein.

In at least one embodiment, a given instance of the Transaction Analysis Procedure may access and/or utilize information from one or more associated databases. In at least one embodiment, at least a portion of the database information may be accessed via communication with one or more local and/or remote memory devices. Examples of different types of data which may be accessed by the Transaction Analysis Procedure may include, but are not limited to, one or more of those described and/or referenced herein.

According to specific embodiments, multiple instances or threads of the Transaction Analysis Procedure may be concurrently implemented and/or initiated via the use of one or more processors and/or other combinations of hardware and/or hardware and software. For example, in at least some embodiments, various aspects, features, and/or functionalities of the Transaction Analysis Procedure may be performed, implemented and/or initiated by one or more of the various systems, components, systems, devices, procedures, processes, etc., described and/or referenced herein, and may be performed asynchronously and substantially concurrently.

According to different embodiments, one or more different threads or instances of the Transaction Analysis Procedure may be initiated in response to detection of one or more conditions or events satisfying one or more different types of minimum threshold criteria for triggering initiation of at least one instance of the Transaction Analysis Procedure . Various examples of conditions or events which may trigger initiation and/or implementation of one or more different threads or instances of the Transaction Analysis Procedure may include, but are not limited to, one or more of those described and/or referenced herein.

According to different embodiments, one or more different threads or instances of the Transaction Analysis Procedure may be initiated and/or implemented manually, automatically, statically, dynamically, concurrently, and/or combinations thereof. Additionally, different instances and/or embodiments of the Transaction Analysis Procedure may be initiated at one or more different time intervals (e.g., during a specific time interval, at regular periodic intervals, at irregular periodic intervals, upon demand)

In at least one embodiment, initial configuration of a given instance of the Transaction Analysis Procedure may be performed using one or more different types of initialization parameters. In at least one embodiment, at least a portion of the initialization parameters may be accessed via communication with one or more local and/or remote memory devices. In at least one embodiment, at least a portion of the initialization parameters provided to an instance of the Transaction Analysis Procedure may correspond to and/or may be derived from the input data/information.

### Contract Ingestion Engine

A Contract Ingestion Engine can be used to intake contracts between service providers and responsible payors. Again, this can be healthcare providers and insurers in the specific relationship disclosed herein for purposes of illustration. It will be appreciated that contracts of many types and formats may exist between various healthcare providers and insurance companies, and such types and formats can include, for example, portable document format (“PDF”) files, Excel files, Word files, and paper contracts, among other types of contracts. Each of these different types of contracts can be input into the system by way of the Contract Ingestion Engine due to the OCR, NLP, and machine learning programs used by the Contract Ingestion Engine. Accordingly, a primary function of the Contract Ingestion Engine is to intake contracts and place relevant contract data in an organized and readily readable fashion into a Contract Rules Database for further use.

FIG. 9 illustrates a flowchart of an exemplary method of processing contract data using a Contract Ingestion Engine. A start event 900 can be triggered, for example, by a contract or contract portion being entered into a raw contract data file database in a remote database system. This can be, for example, Unprocessed Contract Data Database 181 in FIG. 1A. This can involve reacting to raw data being placed into Unprocessed Contact Data Database 181 from anywhere in the system. Alternatively, the system can proactively poll one or more remote clients or system entities to inquire whether new contract data files may be available. After start event 900, contract data can be accessed by the Contract Ingestion Engine at a first process step 902. This can take place at any of various user terminals, as noted above. A PDF or other readable file portion of the contract can be split into individual image pages, and images can be extracted from the PDF using an extraction module, such as, for example, GhostScript. The images can then be fed into an OCR module, such as, for example, an Azure Computer Vision module. A paper contract can be scanned into a PDF format, and this may be of a medium quality, slightly askew scan that is still readable by the OCR module. The input image pages can then be stored for further processing and analysis, which can involve the use of, for example, Microsoft.Net/C#.

At a following process step 904, the accessed contract data can be normalized. This can involve logically organizing text fragments into rows and columns and correcting for any skew where needed. Text elements can be subjected to a machine learning process that categorizes each text element. This can involve, for example, use of an Azure Machine Learning module. The learning process can use a previously trained Multiclass Neural Network model to perform the categorizations. Although specific examples of Azure and Microsoft products and services have been recited for purposes of illustration, it will be appreciated that any other suitable products or services may alternatively be used for the disclosed OCR, parsing, NLP, cloud storage, machine learning, artificial intelligence, augmented reality, virtual reality, cognitive services, network content and delivery, and other computing products and services disclosed herein, and that such alternative products or services can be cloud based, server based, or provided via any other suitable means.

At the next process step 906, the normalized contract data can be parsed. This can involve using the categorized text to guide analysis of the different text items for appropriate contract rule formation and storage. For example, if a text block is categorized as “Amount Per Diem” the system knows to analyze that text block for a currency value and to parse it as a billing rate. As another example, if a text block is categorized as “Nursery Level 1” the system knows to parse it as a service type.

At a following process step 908, pricing information can be identified in the parsed contract data. Pricing information can be particularly useful in the formation of contract rules to be stored on the Contract Rules Database. Pricing can involve several different categories, such as, for example, per diem, per procedure, or per visit, among other possible categories. Contract rules can be formed using the pricing information and various other data items, such as a service, a medical procedure identifier or code (e.g., billing code), copayment and deductible information, healthcare provider, responsible payor, insurance plan type, start date, and end date, among other possible data items.

Formed contract rules can be validated to detect errors in the OCR processing or analysis by the Contract Ingestion Engine. For example, extracted medical procedure codes can be compared to a master list of known allowed medical procedure codes to detect and flag incorrect values. The Contract Ingestion Engine may also analyze past contracts to detect unreasonable fluctuations in pricing amounts and flag for review. In addition, the Contract Ingestion Engine may also analyze contracts and transaction properties to create machine learning insights for future contract data processing.

At the next process step 910, the Contract Rules Database can be populated with the parsed contract data. This can take place after contract rules formed with the parsed contract data are validated. Populating the database can include arranging the parsed contract data into contract rules, which may be arranged into contract rules records, that are then readily accessible by other system components, such as the Payment Reconciliation Engine. The method can then repeat if needed or can end at end event 912. In some embodiments, multiple instances of method 900 can run in parallel. Because the system can constantly monitor for new contact data and then process any new contract data to create and store contract rules accordingly, and may use multiple instantiation agents to process data and create rules in parallel, the system can consistently have a complete set of contract rules that is constantly updated and available for system use when any new claims data is processed. This provides a significant improvement over existing systems that may take weeks or months to update new contracts and may never have an up to date set of contract rules or guidelines for processing new claims data. Further details regarding Contract Ingestion Engine functions and procedures are provided in FIG. 18A below.

For each new contract rule record, one or more Message Bots may be automatically instantiated for one or more of the following (or combinations thereof):
Revenue Code
HCPCS code (Healthcare Common Procedure Coding System)
CPT Code (Common Procedure Technology)
DRG Code (Diagnosis Related Group)
CARC Code (Claim Adjustment Reason Code)
RARC Code (Remittance Advice Remark Code)
Other

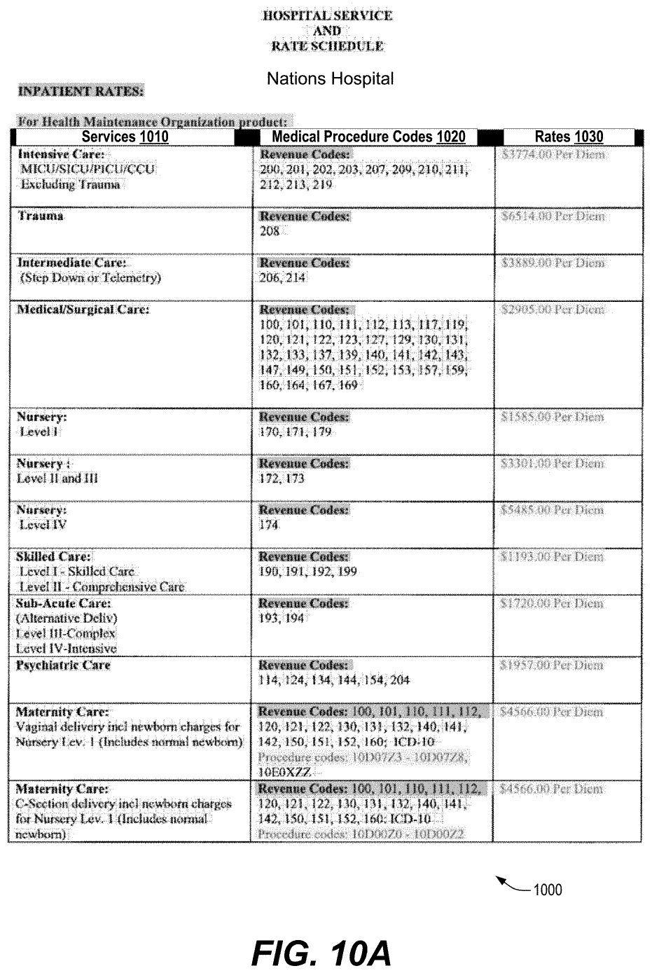

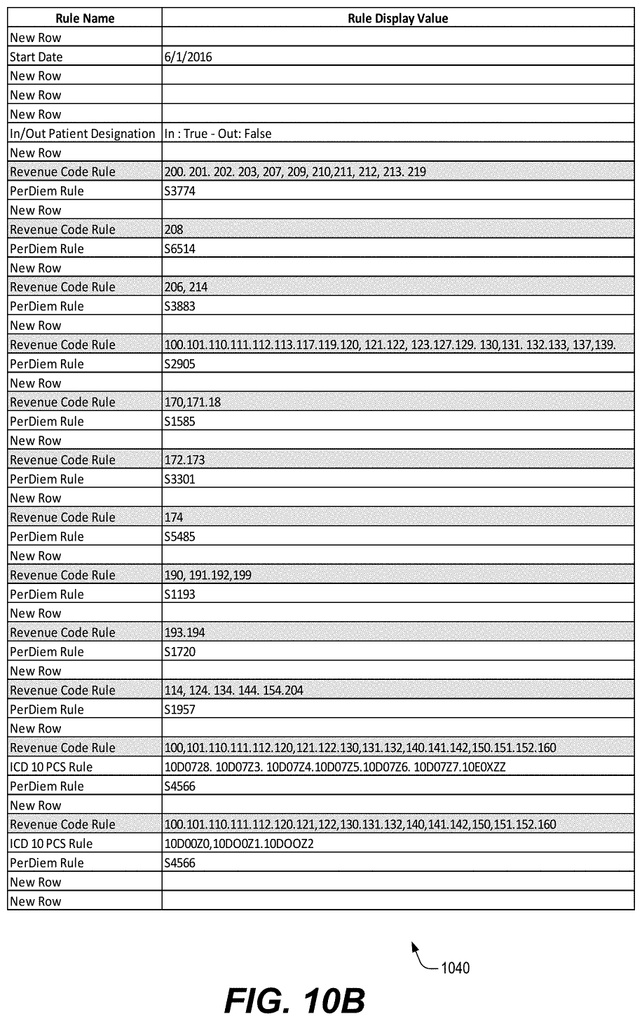

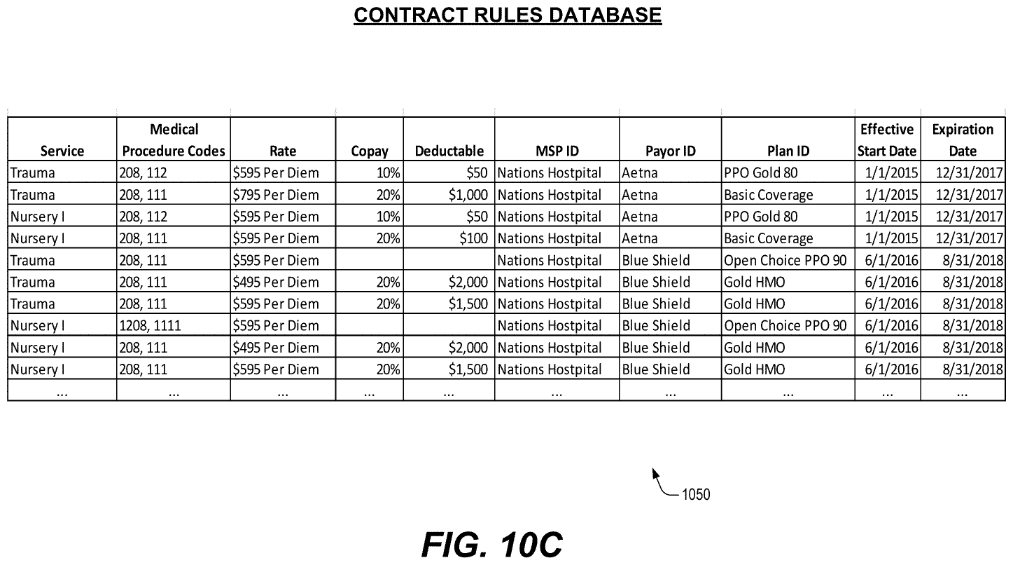

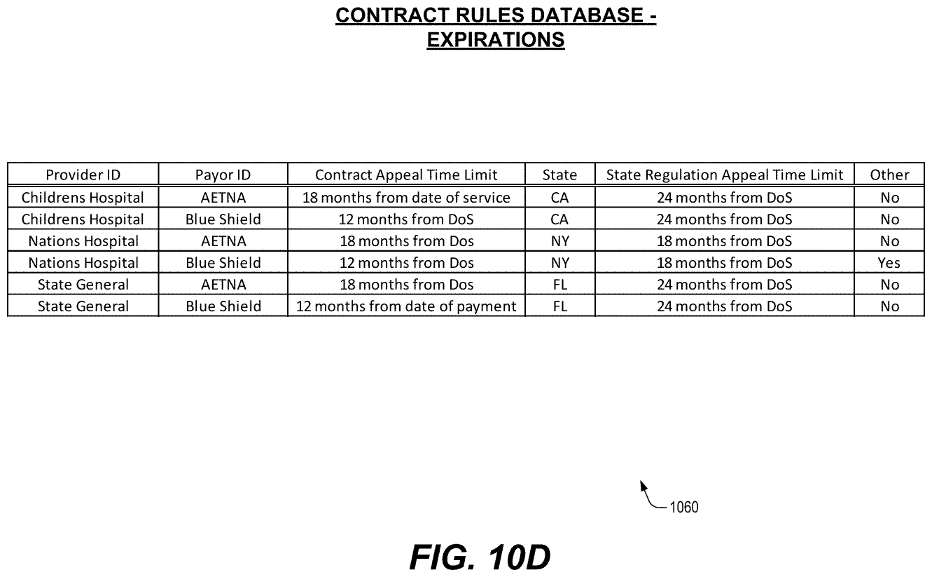

FIG. 10A illustrates an exemplary portion of contract data being parsed by a Contract Ingestion Engine. Contract data portion 1000 can include various data items found in a contract between a given healthcare provider and insurance company payor. These can include, for example, services 1010, medical procedure codes 1020, and payment rates 1030, among other data items. As shown in FIG. 10A, an OCR module is in the process of detecting columns and rows, as indicated by shading on various portions of text. The OCR module can further well recognize text strings in highlighted text and create bounding boxes around them for further processing by NLP and machine learning modules, as will be readily appreciated by those of skill in the art.

FIG. 10B illustrates an exemplary portion of parsed and reorganized contract data by a Contract Ingestion Engine. Contract data portion 1040 can be stored in temporary memory at one or more locations as the Contract Ingestion Engine parses and organizes data into rules before populating the Contract Rules Database with the contract rules. Contract data portion 1040 can include rule names and rule display values as the Contract Ingestion Engine starts to parse data and insert the data into contract rules as may be appropriate. Contract rules can include various data items, such as revenue codes and pricing terms, among other possible data items. Revenue codes may be related to medical procedure codes, and pricing terms may be per diem, per procedure, or per visit, for example.

FIG. 10C illustrates an exemplary portion of contract rules stored on a Contract Rules Database by a Contract Ingestion Engine. Contract rules portion 1050 can include contract rules organized in table format by row, such that each row represents a separate contract rule. Each contract rule can include various different data categories, such as service types, medical procedure codes, payment rates, copayments, deductibles, healthcare providers, payors, payor plans, start dates, and end dates, among other possible data categories. For example, the first contract rule listed can reflect the contract between Nations Hospital and Aetna on a PPO Gold 80 plan from Jan. 1, 2015 to Dec. 31, 2017, and in particular the payment rate of $595 per diem, copayment of 10%, and deductible of $50 for a trauma service under medical procedure codes 208 and 212. In some arrangements, contract rules can be across individual distributed databases that are separated by healthcare provider, payor, or both.

FIG. 10D illustrates another exemplary portion of contract rules stored on a Contract Rules Database by a Contract Ingestion Engine. Different rule types can exist in the Contract Rules Database. For example, the rules shown in FIG. 10C can be a first type of contract rule, while the rules shown in FIG. 10D can be a second type of contract rule. In particular, contract rules portion 1060 can relate to expiration dates with respect to certain medical providers, payors (e.g., insurance companies), state laws and regulations, specific claims, and other factors that may apply. For example, the ability to file an appeal or otherwise recoup revenue for a given claim may expire after a certain time period. This time period can be set forth in a contract between parties, can be specified or limited by state law or regulation, or may be subject to other factors, such as specific details that relate to a given claim or specifics of a given claim Time periods may vary according to each of these and other possible factors. Accordingly, contract rules portion can include appeal expiration dates with respect these and other possible factors, such that data to be recorded in expiration rules can include providers, payors, state regulations, and other factors, as shown. These appeal expiration dates can then be calculated for each claim from the date that service is provided on a claim by claim basis. As noted elsewhere herein, specific claims can be ignored during data processing when the system determines that any ability to appeal that claim has expired.

### Claim Automation Engine

A Claim Automation Engine can be used to receive claim data into the system, particularly for the specific relationship between healthcare providers and insurance companies as disclosed herein. For other types of relational arrangements, a similar engine can be more generically termed a Transaction Automation Engine. As such, a primary function of the Claim Automation Engine is to receive claim data and place it in an organized and readily readable fashion into a Claims Database for further use.

FIG. 11 illustrates a flowchart of an exemplary method of processing claim data using a Claim Automation Engine. A start event 1100 can be triggered by, for example, a claim data file being entered into a raw claim data file database in a database system. This can be, for example, Unprocessed Claims Data Database 183 in FIG. 1A. This can involve reacting to raw data being placed into Unprocessed Claims Data Database 183 from anywhere in the system. Alternatively, the system can proactively poll one or more remote clients or system entities to inquire whether new claim data files may be available. After a start event 1100, claim data can be accessed by the Claim Automation Engine at a process step 1102. This can involve receiving itemized claim invoices and claim payments in the form of 837 and 835 EDI files, for example. Of course, other types of file formats may also be used to hold claim invoices, claim payments, and other claim data, and it is specifically contemplated that appropriate system modules be used to accommodate such other file formats if needed.

At a following process step 1104, the accessed claim data can be normalized. This can involve converting the claim data from an EDI file format as received to a more usable format for further processing, such as, for example, a JSON format. Further details regarding normalizing claim data from and EDI to a JSON format are set forth in FIG. 12 below.

At the next process step 1106, the normalized claim data can be parsed. This can involve recognizing and separating different portions of the normalized claim data by category. Claim data categories can include, but are not limited to, for example, healthcare providers, insurance company payors, patients, services performed, medical procedure codes, payment rate data, and the payor payment amounts, among other claim data items and categories. In addition, various services performed under a single claim for a given patient and patient visit may be delineated by claim line items, such that multiple services performed, medical procedure codes, and payment rate data can fall under the same claim or claim set.

In some arrangements, accessing, normalizing, and/or parsing data may involve only a subset of data from the accessed claim data. For example, different medical providers and/or different payors (e.g., insurance companies) may only require certain parts of data from a claim to be used. This can involve preferences on the part of the parties to the contract data, and/or can involve the service level being provided to a party, such as, for example, the level of service provided to a medical provider based on a service subscription desired by the medical provider. Accordingly, one or more of process steps 1102, 1104, and 1106 may involve using only some of the accessed data. Once data has been accessed, the system (e.g., Claim Automation Engine) can read the data to determine the parties, find service level agreement terms for the parties, and then process only a subset of the data according to the existing service level agreement for the pertinent party or parties. For example, the system can determine the parties, appropriate service level agreement(s), and thereby determine which parts of the claim data are needed and which are not at the accessing data step 1102, such that only the relevant data is normalized and parsed. By processing just the portion of data that is relevant, time and processing capability can be conserved, which can be substantial over the course of processing massive amounts of claims

At the following process step 1108, a Claims Database can be populated with the parsed and normalized claim data. This can involve organizing and formatting the claim data into individual claim records and storing them by claim numbers or other identifiers on the Claims Database. In various arrangements, the claim records can be stored in a manner that allows for ready searching and cross-checking by claim category as well as by individual claim record. The method can then repeat if needed or end at end event 1110. In some embodiments, multiple instances of method 1100 can run in parallel. Further details regarding Claim Automation Engine functions and procedures are provided in FIG. 18B below.

FIG. 12 illustrates a flowchart of an exemplary method of normalizing claim data using a Claim Automation Engine of a computing system according to one embodiment of the present disclosure. A start event can be triggered by, for example, accessing claim data that is in an EDI file format. After start event 1200, an EDI file or data therefrom can be uploaded to a secure file transfer protocol (“SFTP”) server at process step 1202. This can take place at a client, such as by way of a user terminal, for example.

At a following process step 1204, the uploaded EDI file stored at the client can be found by a first function application or module, such as an EDIfindfile module, which can be performed by a findfile application, for example. At the next process step 1206, the first function application can call an EDI to JSON conversion application.

At subsequent process step 1208, which can be performed by a second function application, such as an EDIfileconversion module, the EDI file can be moved to a temporary storage location, such as, for example, an Azure Blob storage location. The remaining process steps can all be performed by the same second function application. Again, a different brand or source of cloud-based storage may alternatively be used.

At the next process step 1210, the EDI file can be converted to a JSON file using the EDI to JSON conversion application. The JSON file can then be saved to the Azure Blob storage or other temporary storage location at process step 1212. At subsequent process step 1214, the JSON file can be stored to a distributed database, such as the Claims Database noted above. In addition to storing the JSON file at the Claims Database, the second function application can format the JSON file so that it is more readily legible to a reviewer or auditor. The method can then repeat if needed or can end at end event 1216. In some embodiments, multiple instances of method 1200 can run in parallel.

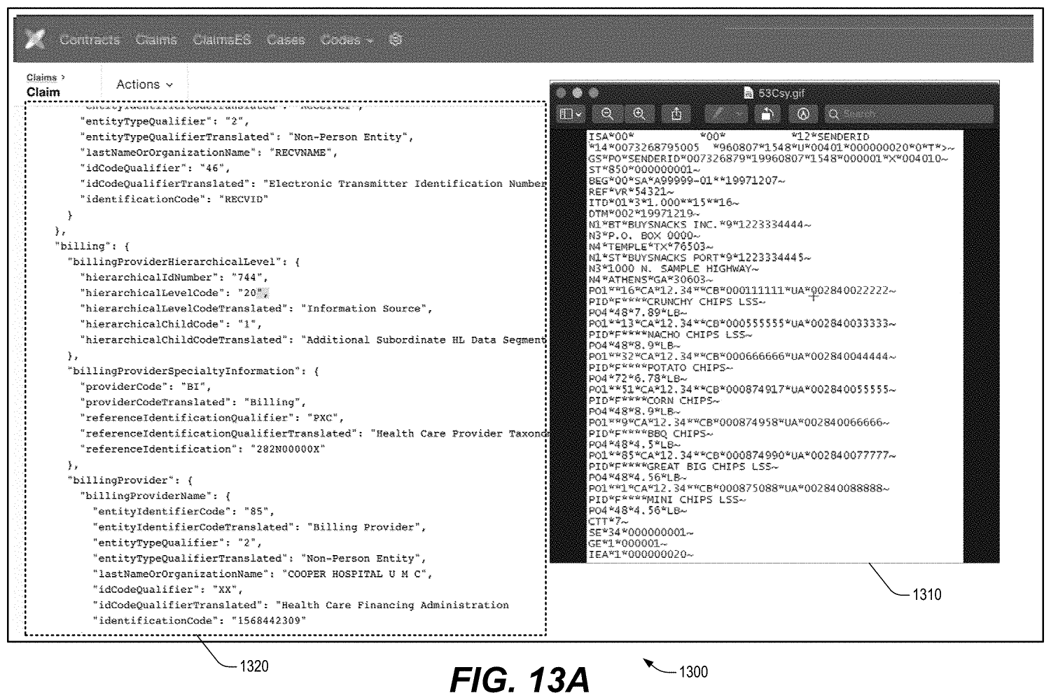

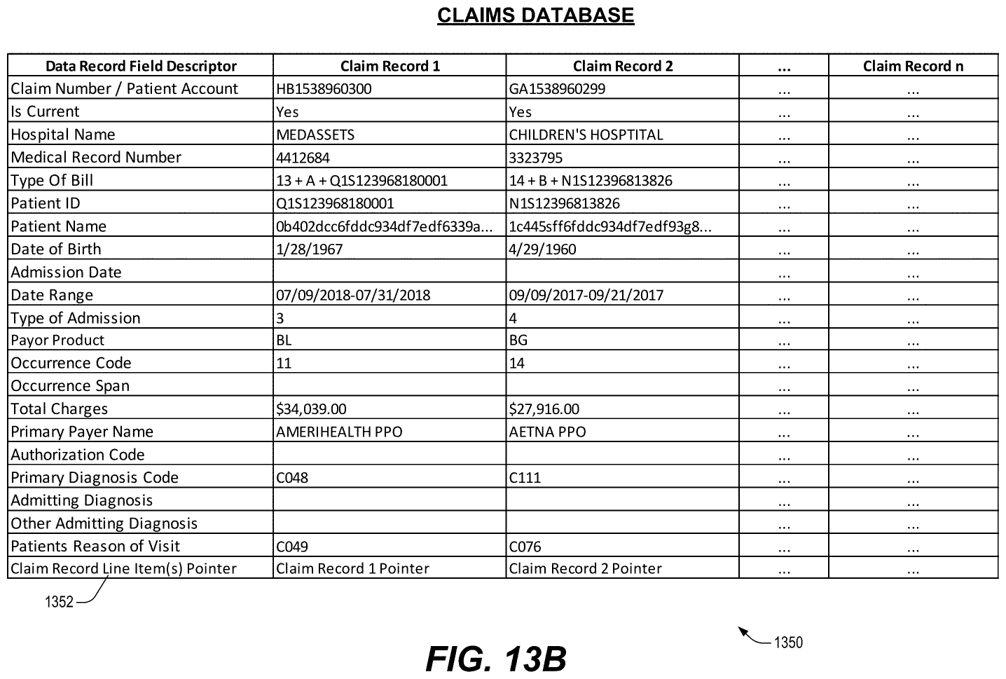

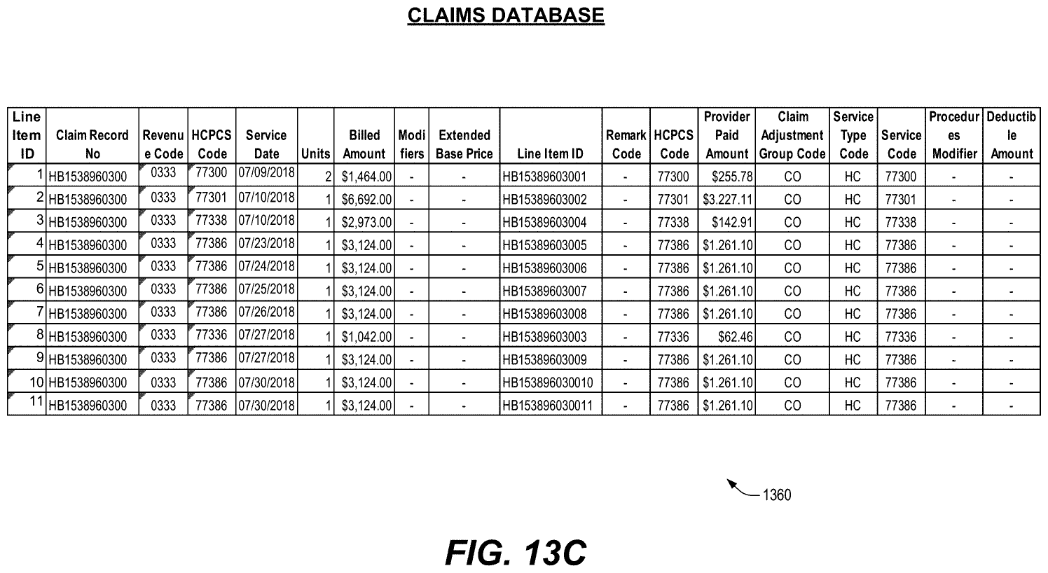

FIG. 13A illustrates an exemplary portion of claims data being normalized and organized by a Claim Automation Engine. Graphical user interface (“GUI”) 1300 shows a first window 1310 that contains claim data in its original EDI format, as well as a second window 1320 that shows the same claim data in its normalized JSON format. As shown, the JSON formatted data has also been organized to be more readily legible by a user, such as a user viewing the claim data on GUI 1300. In various embodiments, the ability to review claim data in both of its EDI and JSON formats as shown may be made available to users for inspection, such as for auditing and error determination purposes.

FIG. 13B illustrates an exemplary portion of parsed and organized claim data by a Claim Automation Engine. Claim records portion 1350 can include separate claim records organized into columns, with data in each of the claim records organized according to data record field descriptors. Each claim record can represent a separate claim for a given patient at a given healthcare provider for a given insurance company payor. Because each claim record can include multiple services and/or goods provided to a patient, claim records can be further categorized by claim line items, which are described in FIG. 13C below. Data record field descriptors for each claim record can include, for example, a claim or patient account number, a status indicator, a healthcare providers, a medical record number, a type of invoice or bill, a patient identifier, a patient name, a patient data of birth, and admission data, a service date range, a type of admission, a payor insurance plan or product, an occurrence code, an occurrence span, total pricing or charges, a payor identifier, an authorization code, a primary diagnosis code, an admitting diagnosis, other diagnoses, and patient reason for visit, It will be understood that not every data field record descriptor listed here must be included, and that other data field record descriptor categories not shown may also be included in a claim record. In addition, a claim record line item pointer 1352 may be associated with each claim record. Such a pointer can assist the system with rapid search and retrieval of data in some instances.

FIG. 13C illustrates an exemplary portion of claim records stored on a Claims Database by a Claims Automation Engine of a computing system according to one embodiment of the present disclosure. Claim record 1360 can include multiple line items, which reflect various services and/or goods provided under the same claim or claim record. As shown in this illustrative example, each of 11 different line items under the same claim record identifier can have various data fields and data entries. Data fields can include, for example, a line item identifier, a claim record number, a revenue code, an HCPCS code, a service date, number of units, a billed amount, modifiers, an extended base price, a line item identifier, a remarks code, a paid amount, a claim adjustment group code, a service type code, a service code, a procedures modifier, and a deductible amount. It will be understood that not every data field category listed here must be included, and that other data field categories not shown may also be included in a claim record.

### Payment Reconciliation Engine

After contract and claim data has been stored to system databases by way of the Contract Ingestion Engine and Claim Automation Engine, the Payment Reconciliation Engine can perform analytics on these various items to perform a variety of functions automatically. These functions can include, for example, detecting billing errors and payment errors, rebilling payors properly or otherwise facilitating recovery of lost assets due to billing or payment errors, creating new contract rules for the contract rules database when similar errors are repeated, instantiating agents to perform asynchronous and substantially concurrent processes on large amounts of data, and analyzing large amounts of data for trends, among other possible functions.

In various embodiments, the Payment Reconciliation Engine can capture at least three data elements for a claim and analyze the data elements according to the stored contract rules that reflect the relevant contract between the provider and payor, such as a healthcare provider and insurance company payor. These three data elements can include, for example, a service provided, a medical procedure identifier, and a payor amount paid for the claim The Payment Reconciliation Engine can also analyze what was actually paid in various similar claims or transactions. Another element can involve determining what a party to a transaction estimated for a given claim or transaction and compare this to what the original payment operation or system expected, which can be compared against the actual billed or invoiced amount as shown in the claim data, as well as the amount expected from data in the Contract Rules Database.

When the Payment Reconciliation Engine identifies a billing error or payment error by revenue diagnosis or otherwise, the system can automatically adjudicate and rebill the payor for repayment of the missing payment amount without human interaction or intervention. These billing or payment errors can include, for example, singular errors for individual cases or claims, as well as identical or substantially similar errors across multiple cases or claims Such identical or similar errors across many cases or claims can then be batched together for a single rebilling to the payor. In various embodiments, the disclosed system can adjudicate up to 80-90% of claim discrepancies without human intervention using this approach.

The Payment Reconciliation Engine can flag an error in the analyzed data, tie an issue code to that error with a tag or flag, for example, and can link tags of issue codes to corresponding codes, for example a benefit code for a hospital or on a payer side. A dynamic analysis component of the Payment Reconciliation Engine can analyze for duplications and unfulfilled requirements for similar claims or transactions. As an example, an insurer can pay one kind of claim in New York for a hospital, which mirrors a transaction for a hospital in California with an identical benefit code. Rather than compounding the error by making the same mistake if a system set up incorrectly due to a local nature of, for example, hospitals and providers, a national paradigm can catch the error(s), including by changing mis-application of codes or tags to right the error on the faulty analysis at the corresponding facility. The Payment Reconciliation Engine can thus tie benefit codes in the software of multiple parties, can fix a systemic mistake that concerns many clients, and can fix many missed or erroneous payments by payors.

In various embodiments, the Payment Reconciliation Engine can include a business intelligence element that can analyze for and spot trends in a variety of areas. For example, it may aggregate lessons from analyzing previous transactions on a payer side of, for example, a hospital system, and on an insurance side of a hospital system. Another Payment Reconciliation Engine feature can involve fraud detection. As an illustrative example in a hospital setting, deeper machine learning insights may identify unexpected returns to surgery or other matters. For example, the Payment Reconciliation Engine can measure the number of patients who must return to surgery unexpectedly because of complications during a surgery and compare this to expected values based on long term data tracking and machine learning techniques. This can result in a prediction as to whether a certain patient has a high risk of encountering an unexpected return to surgery, based on a trained model. A flag can be raised for further inquiry in the event that an unexpected or unlikely event occurs, such as a patient returning to surgery where the risk of such an occurrence was low.

This “unsupervised machine learning” can result in databricks considering all possible data fields for use in prediction. In various arrangements, the Payment Reconciliation Engine can use databricks with the SciKit API, for example, and attempt to choose a model class that conforms to the data. This can include starting with a Naive-Bayesian classification and assessing suitability to the data. Data from one healthcare provider can be used as a training model for one or more other healthcare providers. Data from the next healthcare provider can be used to predict outcomes using the model and validate the predicted outcomes. For example, records from the test healthcare provider can be manipulated to omit records showing a specific event, a prediction using the model can be made to actual patients who encountered a code for that specific event after receiving care, and the machine learning predicted results can then be compared with the actual records when the omitted data is restored. Using this approach, accuracy has been determined to be at above a 90% level.

Extrapolating to other types of relationships and transactions, similar machine learning training steps may be taken in connection with other types of financial transactions, such as in preparation for acquisitions, buyouts, bankruptcy filings, and other matters where due diligence steps seek to determine a more accurate picture of trapped assets before a buyout, or to reduce the risk of learning of a potentially mis-stated reports, or to reduce the risk of fraud Various exemplary procedures that can be performed by the Payment Reconciliation Engine will now be presented at a relatively high level. It will be understood that the Payment Reconciliation Engine may use multiple automated agents to perform some or all of these procedures, as well as multiple instances of the same procedures, asynchronously and substantially concurrently as processing capabilities allow. Further details regarding Payment Reconciliation Engine functions and procedures are provided in FIGS. 18C-18D below.

FIG. 14 illustrates a flowchart of an exemplary method of analyzing transaction data using a Payment Reconciliation Engine. A start event 1400 can be triggered by, for example, a claim data file being entered into a raw claim data file database in a database system. After start event 1400, the Payment Reconciliation Engine can access claim data from the Claims Database at process step 1402. At a process step 1403, a range of claim data to be processed can be determined. Again, this can be based on the level of a service agreement for the parties to the claim data, which may include, for example, a medical provider. The system can determine the parties and the appropriate service level agreement(s), and thereby determine which parts of the claim data are needed and which are not. Data not to be processed can be ignored. For example, data that is too old for appeals to apply according to the contract between the parties can be ignored. As another example, data involving payment amounts or amounts owed that are below a minimum threshold amount according to the relevant service agreement(s) can be ignored. Other rules outside service agreements may also be followed regarding which claims and which data are processed.

At a following process step 1404, the received claim data can be correlated with contract data in the Contract Rules database. At the next process step 1406, payor payments made in the claim data can be compared with pricing information in the contract data. At subsequent process step 1408, discrepancies between payments made and pricing information can be flagged. At the next process step 1410, claim discrepancy data can be generated. At following process step 1412, access to the claim discrepancy data can be provided. The method can then repeat if needed or can end at end event 1414. In some embodiments, multiple instances of method 1400 can run in parallel.

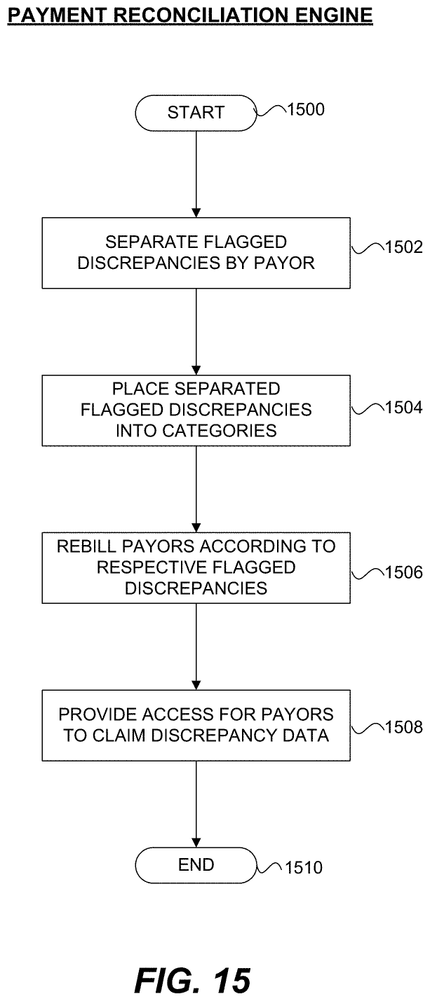

FIG. 15 illustrates a flowchart of an exemplary method of recovering lost assets found using a Payment Reconciliation Engine. A start event 1500 can be triggered by, for example, an instantiation agent initiating a lost asset recovery process. After start event 1500, the Payment Reconciliation Engine can separate flagged discrepancies by payor at process step 1502. At a following process step 1504, the separated flagged discrepancies can be placed into categories. At the next process step 1506, payors can be rebilled according to the respective flagged discrepancies. At subsequent process step 1508, access to the claim discrepancy data is provided to the payors, which can be in a variety of different ways. For example, data can be provided in raw data form, in report form, or as part of an appeal, among other ways of providing data. Delivery of these data items can be automatically provided, such as by online access, by API data, by facsimile, by automated phone calls, by mail, or by electronic mail, among other possible ways of delivering the data. The method can then repeat if needed or can end at end event 1510. In some embodiments, multiple instances of method 1500 can run in parallel.

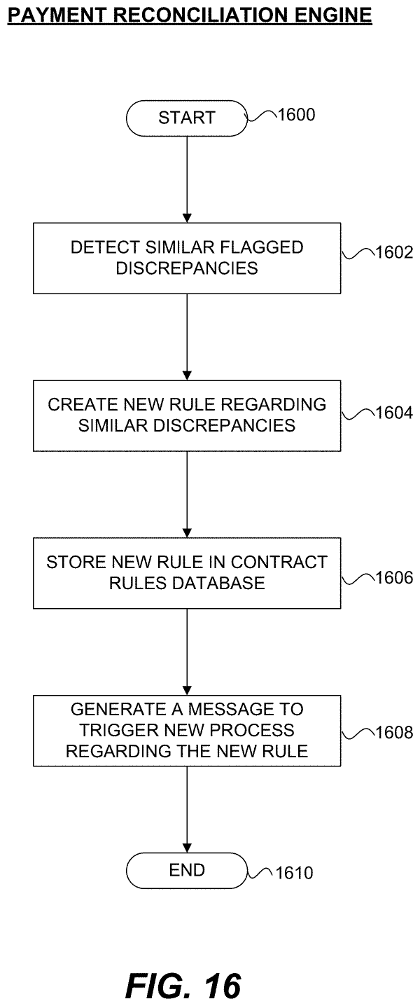

FIG. 16 illustrates a flowchart of an exemplary method of creating a new system rule using a Payment Reconciliation Engine. A start event 1600 can be triggered by, for example, an instantiation agent initiating a new rule creation process. After start event 1600, the Payment Reconciliation Engine can detect similar flagged discrepancies at process step 1602. This can involve, for example, large sets of new data from parties already known to the system, such as existing payors with known issues. At a following process step 1604, a new contract rule regarding the similar discrepancies can be created. At the next process step 1606, the new contract rule can be stored in the Contract Rules Database. At subsequent process step 1608, a message can be generated to trigger a new process regarding the new rule. The method can then repeat if needed or can end at end event 1610. In some embodiments, multiple instances of method 1600 can run in parallel. Again, further details regarding various Payment Reconciliation Engine functions and procedures are provided in FIGS. 18C-18D below.

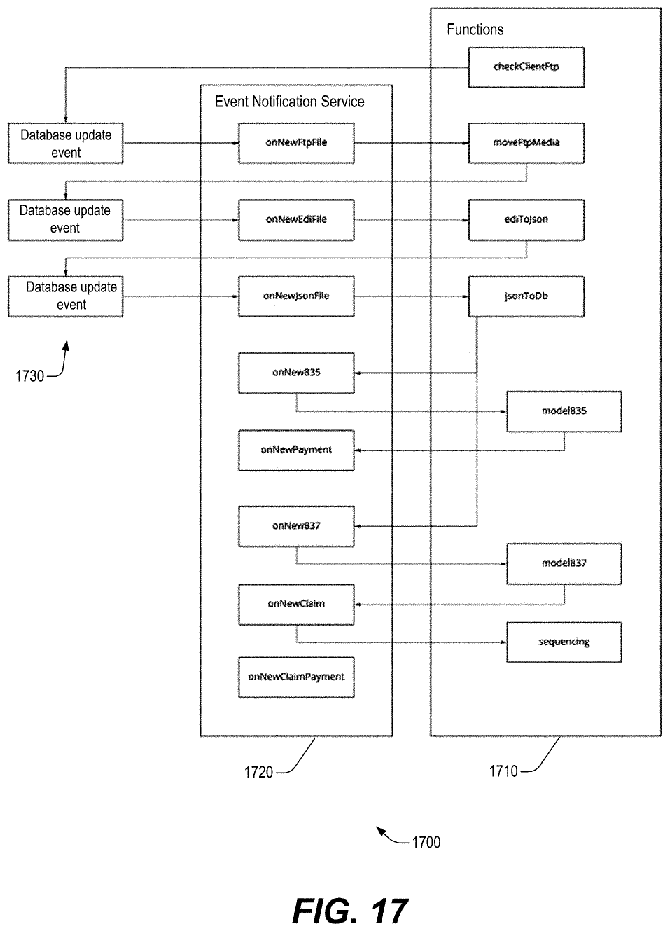

FIG. 17 illustrates a timing diagram of an exemplary new rule message queue for use by a Payment Reconciliation Engine. New rule message queue 1700 can include a stored function stack 1710, a message queue stack 1720, and an event order stack 1730. As an event takes place at event order stack 1730, such as a file delivered to a FTP, an event is generated that adds a message to the message queue stack 1720, which in turn fires off another event that executes a stored stand-alone function at the stored function stack 1710. Stored functions are subscribed to specific message types in the queue and the presence of a new message triggers the function to execute. In general, each item in each stack can be its own event or function, which may be triggered or queued by another item or event. Each stack has a particular order or queue that all items fall into, and whenever an instantiation agent is available it can take the item that is in the front of the queue of the stack to which it is assigned.

FIGS. 18A-18D illustrate more detailed procedural flow diagrams of an exemplary overall detailed method of analyzing and auditing medical claim transactions using a computing system which includes system engines, modules, and components that may operate asynchronously and substantially concurrently according to one embodiment of the present disclosure.

More specifically, FIG. 18A shows a flow diagram of a Contracts Ingestion Procedure 1800 in accordance with a specific embodiment; FIG. 18B shows a flow diagram of a Claim Automation Procedure 1820 in accordance with a specific embodiment; FIG. 18C shows a flow diagram of a Contract Rule Monitoring Procedure 1850 in accordance with a specific embodiment; and FIG. 18D shows a flow diagram of a Payment Reconciliation Procedure 1870 in accordance with a specific embodiment.

According to different embodiments, at least a portion of the various types of functions, operations, actions, and/or other features provided by the computer-implemented procedures of FIGS. 18A-D may be implemented at one or more client systems(s), at one or more server systems (s), and/or combinations thereof.

In at least one embodiment, the computer-implemented procedures of FIGS. 18A-D may be operable to perform and/or implement various types of functions, operations, actions, and/or other features such as one or more of those described and/or referenced herein.

In at least one embodiment, the computer-implemented procedures of FIGS. 18A-D may be operable to utilize and/or generate various different types of data and/or other types of information when performing specific tasks and/or operations. This may include, for example, input data/information and/or output data/information. For example, in at least one embodiment, the computer-implemented procedures of FIGS. 18A-D may be operable to access, process, and/or otherwise utilize information from one or more different types of sources, such as, for example, one or more local and/or remote memories, devices and/or systems. Additionally, in at least one embodiment, the computer-implemented procedures of FIGS. 18A-D may be operable to generate one or more different types of output data/information, which, for example, may be stored in memory of one or more local and/or remote devices and/or systems. Examples of different types of input data/information and/or output data/information which may be accessed and/or utilized by the computer-implemented procedures of FIGS. 18A-D may include, but are not limited to, one or more of those described and/or referenced herein.

In at least one embodiment, one or more instances of the computer-implemented procedures of FIGS. 18A-D may access and/or utilize information from one or more associated databases. In at least one embodiment, at least a portion of the database information may be accessed via communication with one or more local and/or remote memory devices. Examples of different types of data which may be accessed by the computer-implemented procedures of FIGS. 18A-D may include, but are not limited to, one or more of those described and/or referenced herein.

According to specific embodiments, multiple instances or threads of one or more of the computer-implemented procedures of FIGS. 18A-D may be concurrently implemented and/or initiated via the use of one or more processors and/or other combinations of hardware and/or hardware and software. For example, in at least some embodiments, various aspects, features, and/or functionalities of the computer-implemented procedures of FIGS. 18A-D may be performed, implemented and/or initiated by one or more of the various systems, components, systems, devices, procedures, processes, etc., described and/or referenced herein.

According to different embodiments, one or more different threads or instances of the computer-implemented procedures of FIGS. 18A-D may be initiated in response to detection of one or more conditions or events satisfying one or more different types of minimum threshold criteria for triggering initiation of at least one instance of the computer-implemented procedures of FIGS. 18A-D. Various examples of conditions or events which may trigger initiation and/or implementation of one or more different threads or instances of the computer-implemented procedures of FIGS. 18A-D may include, but are not limited to, one or more of those described and/or referenced herein.

According to different embodiments, one or more different threads or instances of the computer-implemented procedures of FIGS. 18A-D may be initiated and/or implemented manually, automatically, statically, dynamically, concurrently, and/or combinations thereof. Additionally, different instances and/or embodiments of the computer-implemented procedures of FIGS. 18A-D may be initiated at one or more different time intervals (e.g., during a specific time interval, at regular periodic intervals, at irregular periodic intervals, upon demand, etc.).

In at least one embodiment, initial configuration of a given instance of the computer-implemented procedures of FIGS. 18A-D may be performed using one or more different types of initialization parameters. In at least one embodiment, at least a portion of the initialization parameters may be accessed via communication with one or more local and/or remote memory devices. In at least one embodiment, at least a portion of the initialization parameters provided to one or more instances of the computer-implemented procedures of FIGS. 18A-D may correspond to and/or may be derived from the input data/information.

In at least one embodiment, one or more of the computer-implemented procedures of FIGS. 18A-D may be implemented at one or more computer systems by causing at least one processor to execute a plurality of instructions stored in non-transient memory.

FIG. 18A shows a flow diagram of a Contracts Ingestion Procedure 1800 in accordance with a specific embodiment. It will be understood that multiple instantiations of Contracts Ingestion Procedure 1800 may take place in parallel and/or simultaneously. For example, a 100 page contract file being processed may involve the use of 100 separate instantiation agents processing that contract file in parallel and/or simultaneously.

As illustrated at 1802, the system may cause at least one processor to execute a plurality of instructions stored in non-transient memory to select a first/next contract data file for processing, the selected contract data file including contract information identifying a first medical service provider and a first payor. This contract data file can be taken from, for example, Unprocessed Contract Data Database 181 in FIG. 1A.

As illustrated at 1804, the system may cause at least one processor to execute a plurality of instructions stored in non-transient memory to process a first/next portion of the selected contract data file using OCR, machine learning, and natural language processing.

As illustrated at 1806, the system may cause at least one processor to execute a plurality of instructions stored in non-transient memory to parse the selected contract data file to identify a first set of procedure/service parameters relating to a medical procedure identifier for an identified medical procedure or service. This can involve, for example, sets located under item 1020 in FIG. 10A, among other possible instances. In at least one embodiment, the medical procedure identifier may be determined using one or more of the following types of information (or combinations thereof): Revenue Code(s); HCPCS code(s) (Healthcare Common Procedure Coding System); CPT Code(s) (Common Procedure Technology Code); DRG Code(s) (Diagnosis Related Group Code); etc.

As illustrated at 1808, the system may cause at least one processor to execute a plurality of instructions stored in non-transient memory to parse the selected contract data file to identify a first set of payment rate parameters associated with the identified medical procedure/service. This can involve, for example, rates located under item 1030 in FIG. 10A, among other possible instances.

As illustrated at 1810, the system may cause at least one processor to execute a plurality of instructions stored in non-transient memory to generate, using the processed contract data, a first contract rule record which includes the first set of procedure/service parameters and the first set of payment rate parameters associated with the identified medical procedure/service.

As illustrated at 1812, the system may cause at least one processor to execute a plurality of instructions stored in non-transient memory to populate a contract rules database with information relating to the first contract rule record.

As illustrated at 1814, the system may cause at least one processor to execute a plurality of instructions stored in non-transient memory to determine if processing has been completed for selected contract data file. In at least one embodiment, if processing has not been completed for the selected contract data file, the system may continue to identify and process additional portions of the selected contract data file in order to identify and generate additional contract rule records.

As illustrated at 1816, when the system has completed or finished contract rule processing of the selected contract data file, it may continue to perform contract rule processing on additional contract rule data files, as desired. Such additional contract data files can be taken from, for example, Unprocessed Contract Data Database 181 in FIG. 1A.

FIG. 18B shows a flow diagram of a Claim Automation Procedure 1820 in accordance with a specific embodiment. It will be understood that multiple instantiations of Claim Automation Procedure 1820 may take place in parallel and/or simultaneously. For example, 50 claims files being processed may involve the use of 50 separate instantiation agents processing all claims files in parallel and/or simultaneously.

As illustrated at 1822, the system may cause at least one processor to execute a plurality of instructions stored in non-transient memory to select first/next claim data file for processing. Such a claim data file can be taken from, for example, Unprocessed Claims Data Database 183 in FIG. 1A.

As illustrated at 1824, the system may cause at least one processor to execute a plurality of instructions stored in non-transient memory to identify first/next claim data record from identified claim data file, the identified claim record including claim record information identifying, for example, a medical service provider, a payer, a patient, and procedure/service line item data associated with the identified claim record. Specific non-limiting examples can be seen at FIGS. 13A-13B, for example.

As illustrated at 1826, the system may cause at least one processor to execute a plurality of instructions stored in non-transient memory to initiate/continue processing of service line item data associated with the identified claim record. Numerous separate line items may be associated with the same claim Specific non-limiting examples of line item data for a claim can be seen at FIG. 13C, for example.

As illustrated at 1827, the system may cause at least one processor to execute a plurality of instructions stored in non-transient memory to populate claim database with claim record data relating to the identified claim record.

As illustrated at 1828, the system may cause at least one processor to execute a plurality of instructions stored in non-transient memory to identify, from the line item record data of the identified claim record, a first/next line item procedure/service associated with the identified claim record.

As illustrated at 1829, the system may cause at least one processor to execute a plurality of instructions stored in non-transient memory to identify, from the line item record data of the identified claim record, a first set of procedure code(s) relating to the identified line item procedure/service. In at least one embodiment, the first set of procedure codes may include at least one Revenue code and/or at least one HCPCS code.

As illustrated at 1830, the system may cause at least one processor to execute a plurality of instructions stored in non-transient memory to identify, from the line item record data of the identified claim record, a Provider billed amount relating to the identified line item procedure/service.

As illustrated at 1832, the system may cause at least one processor to execute a plurality of instructions stored in non-transient memory to identify, from the line item record data of the identified claim record, a Payor paid amount relating to the identified line item procedure/service.

As illustrated at 1834, the system may cause at least one processor to execute a plurality of instructions stored in non-transient memory to identify, from the line item record data of the identified claim record, other claim-related information associated with the identified line item procedure/service.

As illustrated at 1836, the system may cause at least one processor to execute a plurality of instructions stored in non-transient memory to populate claim database with line item record data relating to the identified line item procedure/service. Create association between line item data record and identified claim record. In at least one embodiment, the line item record data may include information identifying one or more Claim Adjustment Group Code(s) associated with that line item data record (e.g., as illustrated at item 1352 in FIG. 13B and to FIG. 13C).

As illustrated at 1838, the system may cause at least one processor to execute a plurality of instructions stored in non-transient memory to determine if processing has been completed for identified claim record. In at least one embodiment, if processing has not been completed for the identified claim record, the system may continue to identify and process additional line items associated with the identified claim record. Accordingly, steps 1828-1838 can be repeated until all claim line items for the claim have been processed.

As illustrated at 1840, the system may cause at least one processor to execute a plurality of instructions stored in non-transient memory to determine if processing has been completed for identified data file. In at least one embodiment, if processing has not been completed for the identified claim data file, the system may continue to identify and process additional claim records from the current claim data file. Accordingly, steps 1824-1840 can be repeated until all claims in the data file have been processed.

As illustrated at 1842, when the system has completed or finished claim analysis and processing of the current claim data file, it may continue to perform claim analysis and processing on additional claim data files, as desired. Accordingly, steps 1822-1842 can be repeated until all claim data files have been processed.

FIG. 18C shows a flow diagram of a Contract Rule Monitoring Procedure 1850 in accordance with a specific embodiment. It will be understood that similar procedures or portions thereof can be done in parallel with and with respect to the procedures of FIGS. 18A and/or 18B above. Again, this can involve the use of multiple instantiation agents to perform multiple processes in parallel and/or simultaneously.

As illustrated at 1852, the system may cause at least one processor to execute a plurality of instructions stored in non-transient memory to monitor contract rules database for rule update event(s), the rule update event(s) relating to detection of a new or modified rule record at the contract rules database.

As illustrated at 1854, the system may cause at least one processor to execute a plurality of instructions stored in non-transient memory to detect a rule update event relating to an identified contract rule record in the contract rules database.

As illustrated at 1856, in response to detecting a rule update event, the system may cause at least one processor to execute a plurality of instructions stored in non-transient memory to analyze the identified contract rule record to identify contract rule parameters associated with the identified contract rule record.

As illustrated at 1858, the system may cause at least one processor to execute a plurality of instructions stored in non-transient memory to instantiate and configure at least one automated agent process (e.g., message broker) to perform payment reconciliation analysis on a first set of claim records using the identified contract rule parameters. In at least one embodiment, the automated agents may each perform their respective payment reconciliation analysis via execution of respective instances of the Payment Reconciliation Procedure 1870 (FIG. 18D).

According to different embodiments, one or more separate automated agents may be independently instantiated based on one or more of the following types of data (or combinations thereof) included in the identified contract rule record: Revenue Code(s); HCPCS Code(s) (Healthcare Common Procedure Coding System); CPT Code(s) (Common Procedure Technology Code); DRG Code(s) (Diagnosis Related Group Code); CARC Code(s) (Claim Adjustment Reason Code); RARC Code(s) (Remittance Advice Remark Codes); etc.

As illustrated at 1860, 1662, any errors which are detected may be handled by automatically initiating appropriate error handling procedures.

As illustrated at 1864, the Contract Rule Monitoring Procedure may be configured or designed to continue to monitor the contract rules database for additional rule update event(s), if desired.

FIG. 18D shows a flow diagram of a Payment Reconciliation Procedure 1870 in accordance with a specific embodiment.

As illustrated at 1872, it is assumed that an automated agent has initiated an instance of the Payment Reconciliation Procedure to perform payment reconciliation analysis on a first set of claim records using a first set of contract rule parameters associated with a first contracts rule record. The first set of contract rule parameters can be predetermined, can be determined by the system independently of Payment Reconciliation Procedure 1870, and/or may occur prior to or in parallel with the procedure. The first set of contract rule parameters can be determined based on a variety of factors. For example, this can be based on the level of a service agreement for the parties to the claim data, which may include, for example, a medical provider, and/or an insurance company or other payor. The system can determine the parties and the appropriate service level agreement(s), and thereby determine which parts of the data are needed and which are not for the first contracts rule record. Data not to be processed can be ignored. For example, data involving payment amounts or amounts owed that are below a minimum threshold amount according to the relevant service agreement(s) can be ignored.

As another example, data that is too old for appeals to apply can be ignored. Date ranges for whether appeal can still apply may be based on a contract between the relevant parties, on state regulation rules, or on other factors. Data can be separated into two categories, such as records that are appealable and records for which the possibility of appeal has expired. Data regarding whether a claim appeal possibility has expired can be checked by data in the system and contract expiration rules that may be stored in the Contract Rules Database, such as that which is shown in FIG. 10D. Accordingly, an instantiation agent can initially check against an expiration type of rule stored in the Contract Rules Database to determine whether a given claim should even be further processed. Other rules outside service agreements may also be followed regarding which claims and which data are processed.

As illustrated at 1874, the system may cause at least one processor to execute a plurality of instructions stored in non-transient memory to identify the first set of contract rule parameters associated with an identified contracts rule record representing an identified contracts rule, the first set of contract rule parameters including a first procedure/service parameter relating to an identified medical procedure or service, the first set of contract rule parameters further including a first Payor payment rate parameter associated with the identified medical procedure/service.

As illustrated at 1876, the system may cause at least one processor to execute a plurality of instructions stored in non-transient memory to Identify first/next claim record of the first set of claim records for payment reconciliation analysis.

As illustrated at 1878, the system may cause at least one processor to execute a plurality of instructions stored in non-transient memory to identify a first/next claim record line item associated with the identified claim record, the identified claim record line item including Provider service/procedure information identifying a first medical procedure or service, the identified claim record line item further including a billed amount representing an amount billed by the Provider for the identified medical procedure or service, the identified claim record line item further including a Payor paid amount representing an amount payed by the Payor for the identified medical procedure or service.

As illustrated at 1880, the system may cause at least one processor to execute a plurality of instructions stored in non-transient memory to determine if the first set of contract rule parameters of the identified contracts rule apply to the identified claim record line item. In at least one embodiment, such a determination may involve searching, identifying, comparing and/or matching one or more medical procedure identifiers (e.g., Revenue Code(s); HCPCS Code(s)) identified in the identified contracts rule record and claim record line item.

As illustrated at 1882, in response to determining that the first set of contract rule parameters of the identified contracts rule apply to the identified claim record line item, the system may cause at least one processor to execute a plurality of instructions stored in non-transient memory to calculate an expected contract payment amount for the identified claim record line item using the first set of contract rule parameters and data associated with the identified claim record line item.

As illustrated at 1884, the system may cause at least one processor to execute a plurality of instructions stored in non-transient memory to compare the expected contract payment amount to the Payor paid amount associated with the identified claim record line item.

As illustrated at 1886, the system may cause at least one processor to execute a plurality of instructions stored in non-transient memory to determine if there exists a payment discrepancy between the expected contract payment amount and Payor paid amount.

As illustrated at 1888, in response to identifying a payment discrepancy between the expected contract payment amount and Payor paid amount, the system may cause at least one processor to execute a plurality of instructions stored in non-transient memory to calculate a claim record discrepancy amount, wherein the discrepancy amount represents a difference between the expected contract payment amount and the Payor paid amount associated with the identified claim record line item. The expected contract payment amount can be calculated based on what is allowed according to the relevant contract, terms or rules for which can be stored in the Contract Rules Database and can apply to every relevant line item in a given claim

As illustrated at 1890, the system may cause at least one processor to execute a plurality of instructions stored in non-transient memory to determine if processing has been completed for identified claim record. In at least one embodiment, if processing has not been completed for the identified claim record, the system may continue to identify and process additional line items associated with the identified claim record.

As illustrated at 1892, when the system has completed or finished analysis and processing of the current claim record, it may continue to perform payment reconciliation analysis on additional claim records of the first set of claim records, as desired.

It will be appreciated that different embodiments of the computer-implemented procedures of FIGS. 18A-D (not shown) may include additional features and/or operations than those illustrated in the specific embodiments of FIGS. 18A-D, and/or may omit at least a portion of the features and/or operations of the computer-implemented procedures of FIGS. 18A-D.

### System Front End

The system front end can include various user devices, portals, terminals, and other access points, as noted above. In various embodiments, the system can be accessed from a variety of devices, such as dedicated terminal stations, desktop computers, laptop computers, tablets, smart phones, and other personal electronic devices. In various arrangements, an app such as the “TransactionAnalytics” app noted above may be used on smart phones for users to access the system and utilized various functions and features of the system. In these arrangements, a customized GUI or set of customized GUIs can be provided for users to have various ways of using the system regardless of the exact device being used for system access.

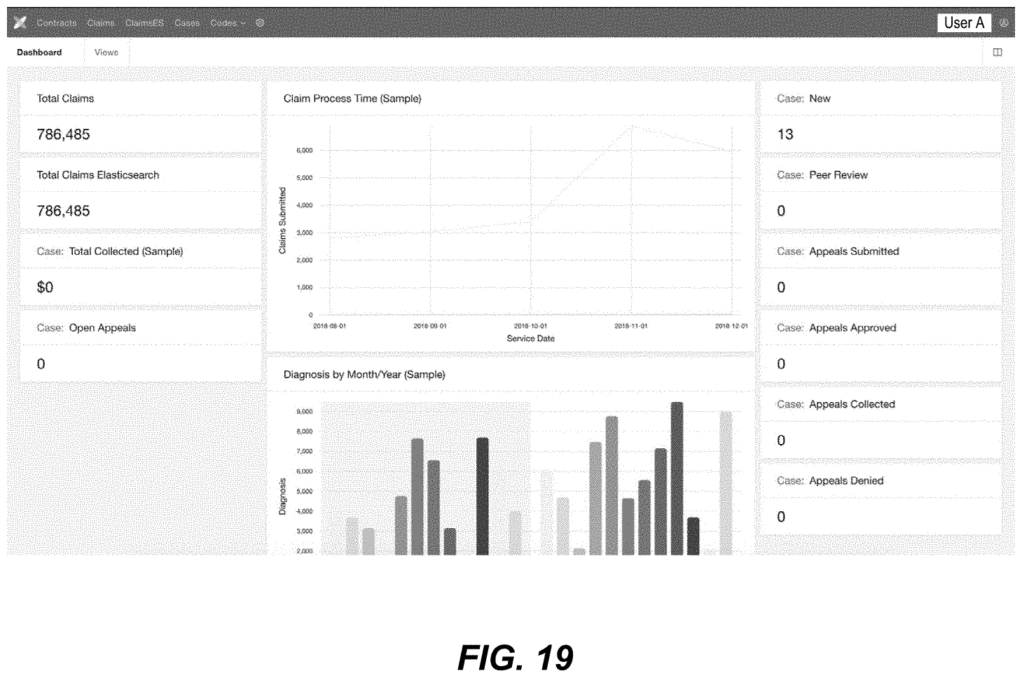

FIGS. 19-55 illustrate screenshots of an exemplary GUI that may be used to facilitate, initiate and/or perform various operation(s) and/or action(s) relating to the disclosed transaction analysis and asset recovery systems and overall Transaction Analysis Procedure. The particular displays provided by the provided front end GUI can depend upon user and developer inputs, as well as available system data. Regular user and developer operations and actions available in conjunction with the system GUI can include, for example, at least one or more of the following (or combinations thereof):
view summaries on dashboard pages (e.g., upon login)
customize data displayed on dashboard pages
select different sections and pages from readily available selection bars
view claim listings by claim numbers and other identifying categories and filters
create read update delete (“CRUD”) any data based on permissions
view cases created from discrepancies
case management processing (generate appeal letters, comments, status, etc.)
manage global variables and lists (CPT codes, users, clients, payors, etc.)
view and manage contracts rules
other

In the specific embodiment of FIG. 19, it is assumed that a user has used the system GUI to display an initial dashboard home page having data displays, charts, and summaries for various system items and categories. This can take place, for example, by a user selecting a “home” button or icon from within various pages within the overall system GUI. Alternatively, the user might be able to input a keyboard shortcut (e.g., CTL+H) to arrive at the same initial dashboard home page. The initial dashboard home page can be customized to present data and information in a manner preferred by a given user. Data and information on this GUI page can include, for example, total claims, total claims available for search, total collections, open appeals, claim process times, diagnoses over time, number of new cases, number of peer reviewed cases, number of case appeals submitted, number of case appeals approved, number of case appeals collected, and number of case appeals denied, among other possible data categories. A regular user accessing this GUI page can get an account snapshot with key performance indicators such as claims, collection details, case status, and the like. The account is displayed on the top right next to the profile icon in the blue bar. A developer accessing this GUI page can aggregate detailed reports about the account and serve regular users with metrics upon login on a home dashboard so users can get an understanding on various account statuses.

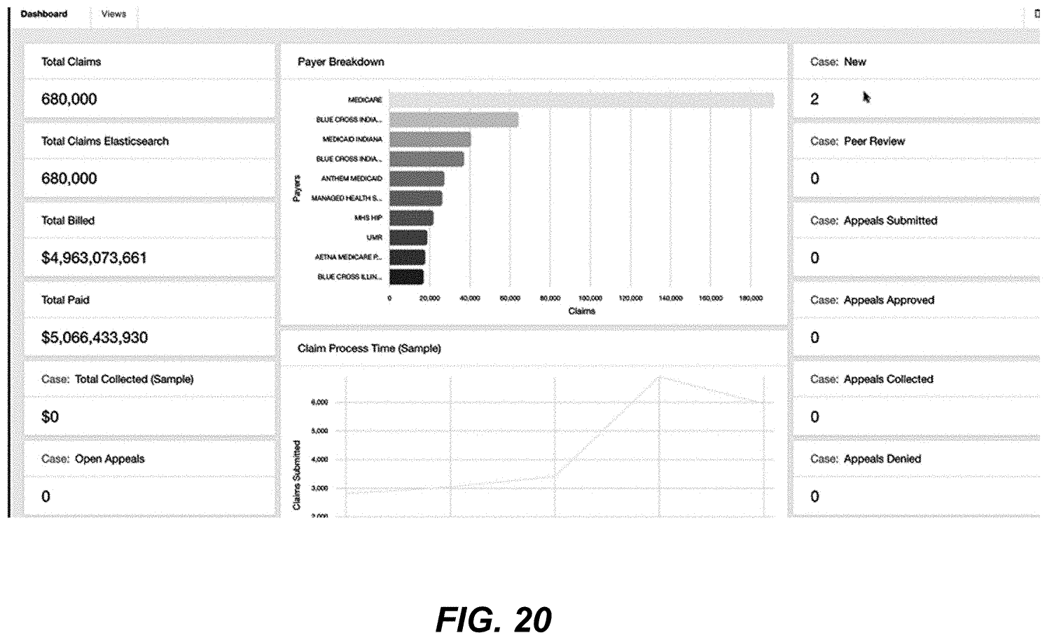

In the specific embodiment of FIG. 20, it is assumed that a user has used the system GUI to display a secondary dashboard page having further data displays, charts, and summaries for various system items and categories. This can take place, for example, by a user selecting a toggle page function within the GUI, such as a selection under a “Views” tab next to the “Dashboard” header. Alternatively, the user can input a keyboard shortcut (e.g., CTL+PGDN) to arrive at the same secondary dashboard page. This secondary dashboard page can also be customized to present data and information in a manner preferred by a given user. Data and information on this GUI page can include, for example, total claims, total claims available for search, total amounts billed, total amounts paid, case total collected, open appeals, claim breakdowns by payer, claim process times, number of new cases, number of peer reviewed cases, number of case appeals submitted, number of case appeals approved, number of case appeals collected, and number of case appeals denied, among other possible data categories.

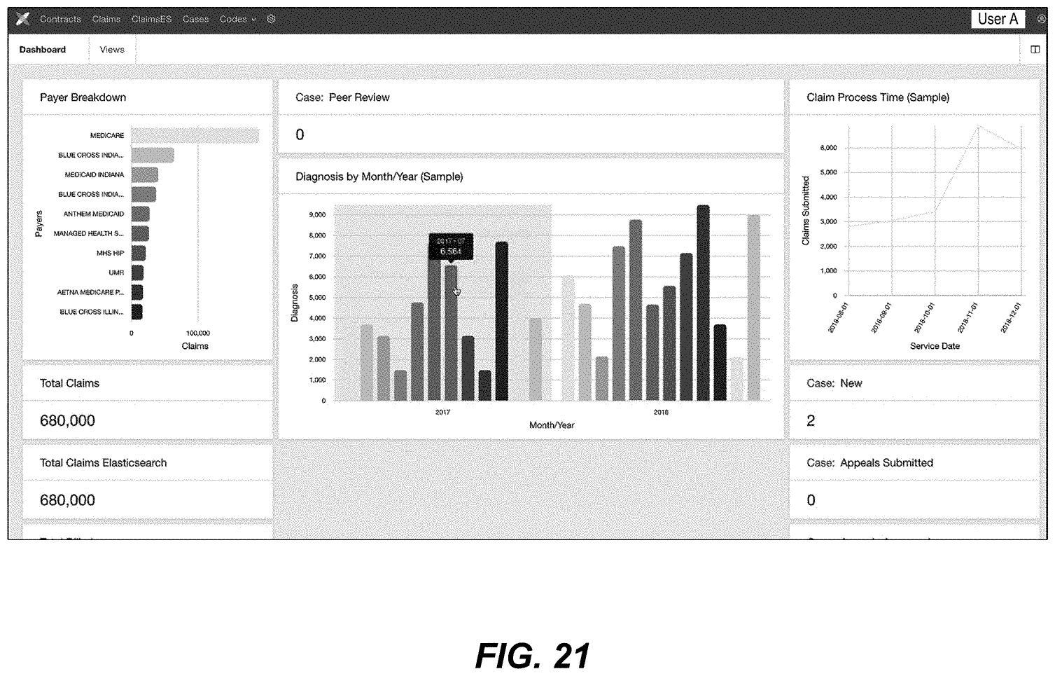

In the specific embodiment of FIG. 21, it is assumed that a user has used the system GUI to display a tertiary dashboard page having further data displays, charts, and summaries for various system items and categories. This can also take place, for example, by a user selecting a toggle page function within the GUI, such as a selection under a “Views” tab next to the “Dashboard” header. Alternatively, the user can input a keyboard shortcut (e.g., CTL+PGUP) to arrive at the same tertiary dashboard page. This tertiary dashboard page can also be customized to present data and information in a manner preferred by a given user. Data and information on this GUI page can include, for example, a breakdown of claims by payer, total claims, total claims available for search, total amounts billed, diagnoses amounts by month and year, number of new cases, number of peer reviewed cases, number of case appeals submitted, number of case appeals approved, number of case appeals collected, and number of case appeals denied, among other possible data categories.

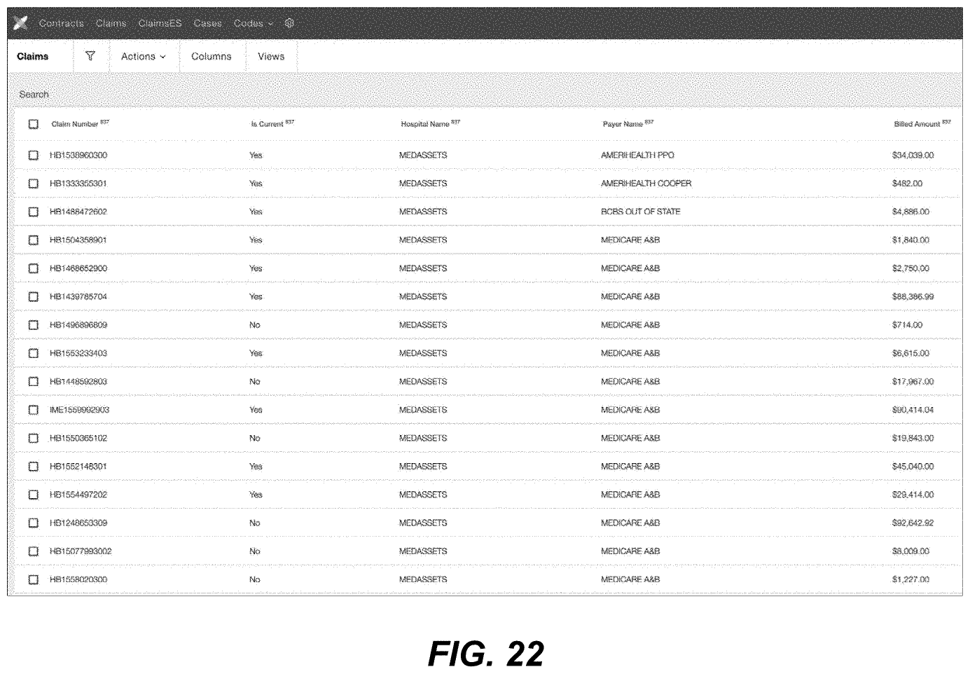

In the specific embodiment of FIG. 22, it is assumed that a user has used the system GUI to display a Claims page that lists individual claims, such as by claim number. This can take place, for example, by a user selecting a “Claims” tab at a top selection bar within the GUI. Alternatively, the user can input a designated keyboard shortcut to arrive at the same Claims listing page. Data and information on this GUI page can include, for example, individual claims listed by claim number, with additional information for each listed claim including current status, hospital name, payer name, and billed amount. Each separate listed claim may be selected to bring up further details regarding that claim A regular user accessing this GUI page can browse some or all claims in an account with a simple grid view. A developer accessing this GUI page can give users the ability to review a listing of claims in a grid view as shown.

In the specific embodiment of FIG. 23, it is assumed that a user has used the system GUI to display a primary filters pop up box with different data fields to allow a user to search through a large number of claims This can take place, for example, by a user selecting a filtering icon next to the Claims header within a Claims page on the GUI. Alternatively, the user can input a designated keyboard shortcut to arrive at the same filters pop up box. Data fields on this primary filter pop up box can include, for example, claim number, hospital name, payer name, and billed amounts. Clear and Apply buttons can assist the user with filling in and applying data to be used in filtering a desired search.

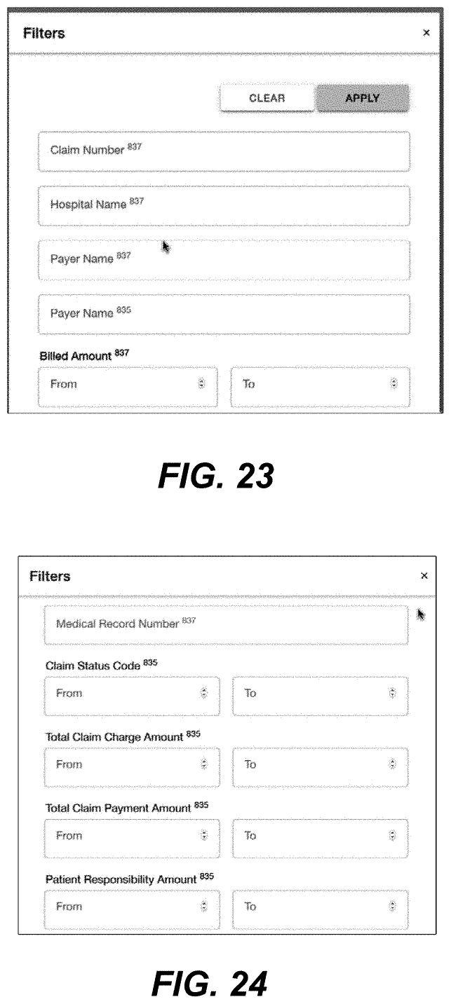

In each of the specific embodiments of FIGS. 24-28, it is assumed that a user has used the system GUI to display a respective further filters pop up box with further data fields to allow a user to search through a large number of claims This can take place, for example, by a user selecting a filtering icon next to the Claims header within a Claims page on the GUI and selecting a respective further filtered data field from a drop down menu on the filtering icon. Alternatively, the user can input a designated keyboard shortcut to arrive at the same respective further filters pop up box.

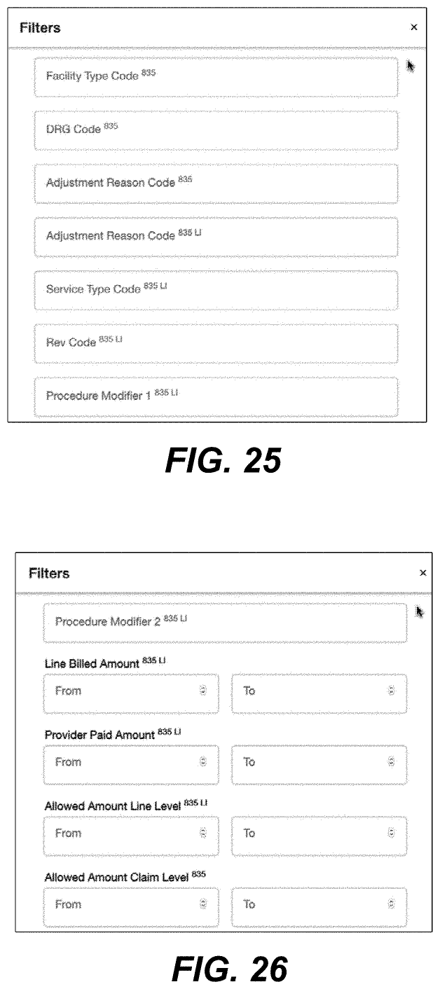

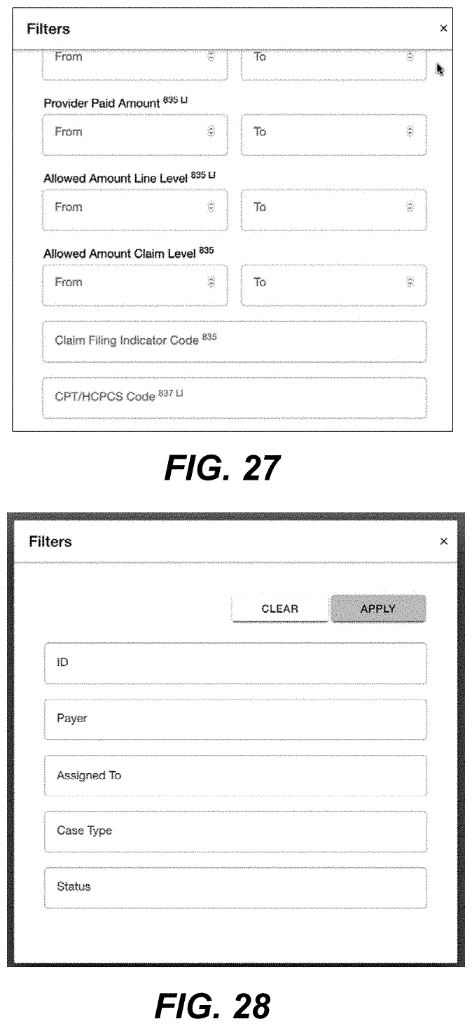

Data fields on the further filters pop up box for FIG. 24 can include, for example, medical record number, claim status codes, total claim charge amounts, total claim payment amounts, and patient responsibility amounts. Data fields on the further filters pop up box for FIG. 25 can include, for example, facility type code, DRG code, adjustment reason code per entire claim, adjustment reason code per claim line item, service type code, revenue code, and a first procedure modifier. Data fields on the further filters pop up box for FIG. 26 can include, for example, a second procedure modifier, line billed amounts, provider paid amounts, allowed amounts at a claim line item level, and allowed amounts at an entire claim level. Data fields on the further filters pop up box for FIG. 27 can include, for example, provider paid amounts, allowed amounts at a claim line item level, allowed amounts at an entire claim level, claim filing indicator code, and CPT/HCPCS code. Data fields on the further filters pop up box for FIG. 28 can include, for example, ID, payer, assigned to, case type, and status. Clear and Apply buttons can assist the user with filling in and applying data to be used in filtering a desired search.

Alternatively, each of the filter pop up boxes shown in FIGS. 23-28 can be provided in series with a single selection of the filtering icon. Moving between the various data fields can simply involve scrolling up or down within the currently displayed filter pop up box. A regular user accessing any of these GUI filter pop up boxes can filter a search to provide specific claims in case view. A developer accessing any of these GUI filter pop up boxes can allow users to sort and filter to specific cases that are applicable.

In the specific embodiment of FIG. 29, it is assumed that a user has used the system GUI to display a columns pop up box with different drop down menu selections to allow a user to select column categories for display when displaying a list of claims This can take place, for example, by a user selecting a “Columns” input next to the Claims and filter icon header within a Claims page on the GUI. Alternatively, the user can input a designated keyboard shortcut to arrive at the same columns pop up box. Drop down menu selections on the columns pop up box can include, for example, ID, payer, projected amount due, collected amount, assigned to, case type, and status. A regular user accessing this GUI pop up box can select which columns to be shown in a displayed grid of data. A developer accessing this GUI pop up box can provide users with abilities to customize their views with the data elements applicable for their particular roles. In this manner, the system can allow regular users to customize the data that they are able to view without requiring developers to create different views for each user, such that the system delivers a quality user experience for many different roles.

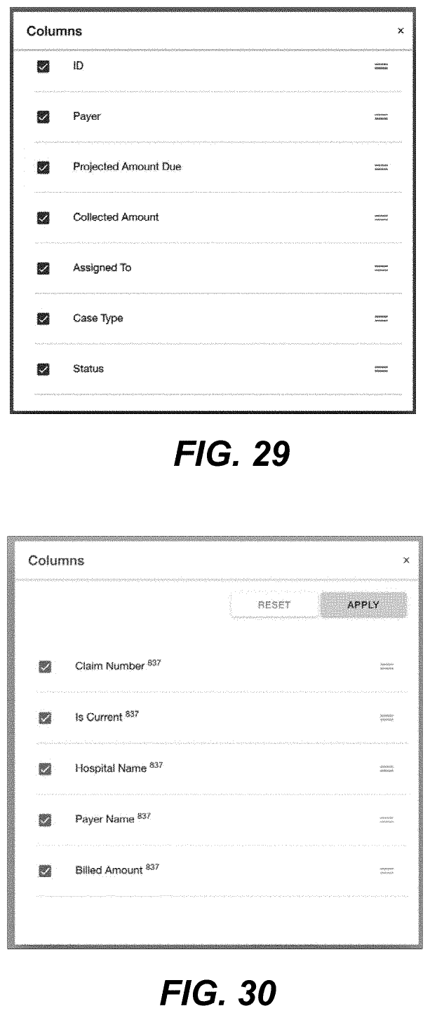

In the specific embodiment of FIG. 30, it is assumed that a user has used the system GUI to display a secondary columns pop up box with different drop down menu selections to allow a user to select column categories for display when displaying a list of claims user to select various filters by column to facilitate a search through a large number of claims This can take place, for example, by a user selecting a “Columns” input next to the Claims and filter icon header within a Claims page on the GUI and then selecting a respective further columns option from a drop down menu on the “Columns” input. Alternatively, the user can input a keyboard shortcut to arrive at the same secondary columns pop up box. Also alternatively, each of the columns pop up boxes shown in FIGS. 29-30 can be provided in series with a single selection of the “Columns” input. Moving between the various selections can simply involve scrolling up or down within the currently displayed columns pop up box. Data and information on this secondary column pop up box can include, for example, claim number, current status, hospital name, payer name, and billed amount. Reset and Apply buttons can assist the user with making selections and applying selections to be used in displaying desired columns.

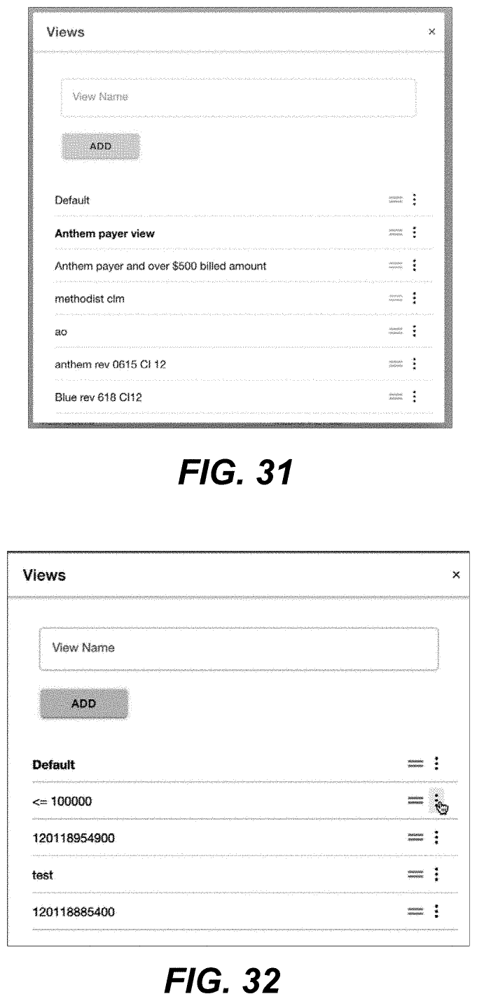

In the specific embodiments of FIGS. 31-32, it is assumed that a user has used the system GUI to display a views pop up box with different categorical menu selections to allow a user to select various data view categories for display when displaying a list of claims This can take place, for example, by a user selecting a “Views” input next to the Claims, filter icon, and Columns header within a Claims page on the GUI. Alternatively, the user can input a designated keyboard shortcut to arrive at the same views pop up box. Categorical menu selections on the views pop up box can include, for example, default views, as well as customized view categories created by an individual user, which may be by payer name, payer name and minimum billed about, hospital name, payer name and revenue code, a less than billed amount, a specific claim number, a test category, and any other customized combination a user might desire. An Add button can assist the user in saving a customized view for later use. A scroll feature can allow a user to toggle between a list of different customized view categories, such as the differently displayed views pop up boxes of FIGS. 31 and 32. A regular user accessing this views pop up box can save customized filters views that can be rerun at any time in the future without having to spend the time to refilter. A developer accessing this views pop up box can allow users to save their workloads and reapply them to future views.

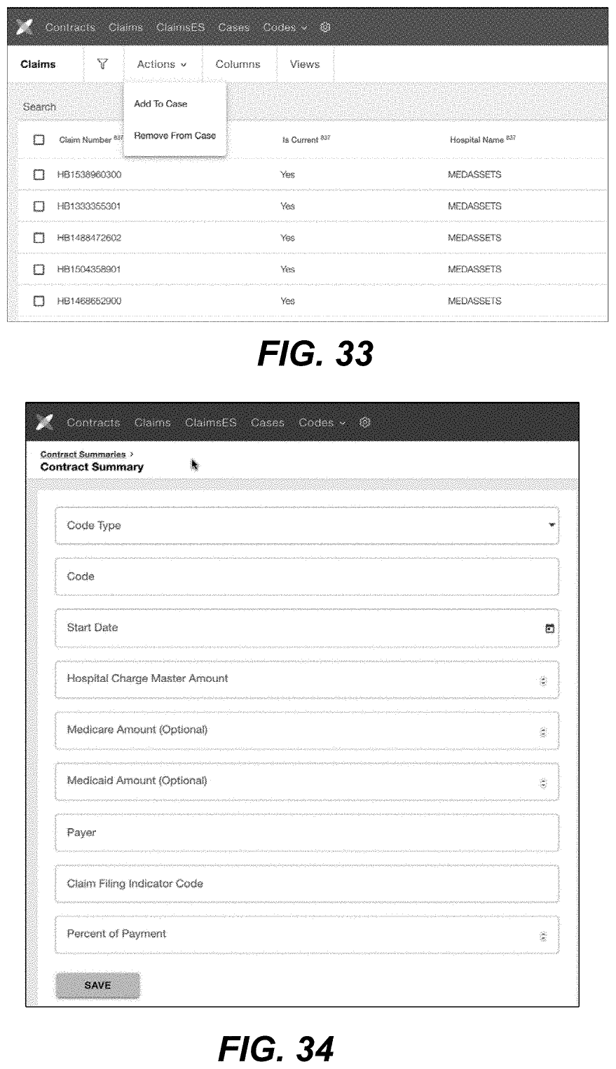

In the specific embodiment of FIG. 33, it is assumed that a user has used the system GUI to display an Actions menu on a Claims page that lists individual claims, such as by claim number. This can take place, for example, by a user selecting a “Claims” tab at a top selection bar within the GUI and then selecting an Actions drop down menu along a page header. Alternatively, the user can input a designated keyboard shortcut to arrive at the same Actions menu on a Claims listing page. Available actions on the drop down Actions menu can include add to case and remove from case, among other possible available actions. A regular user accessing this GUI page and drop down menu can take a customized filtered view of claims and perform actions to add claims to a case or remove from a case. A developer accessing this GUI page can give the user ability to manage all claims and associations to cases.

In the specific embodiment of FIG. 34, it is assumed that a user has used the system GUI to display a Contract Summary page having data entry fields to allow searches for specific contracts in the system. This can take place, for example, by a user selecting a “Contracts” tab at a top selection bar within the GUI. Alternatively, the user can input a keyboard shortcut to arrive at the same Contract Summary page. Data fields to aid in contract searches on this GUI page can include, for example, a code type from various available code type possibilities, start date, hospital charge master amount, Medicare amount, Medicaid amount, payer, claim filing indicator code, and percent of payment. A Save button can assist the user in saving specific terms for future searching.

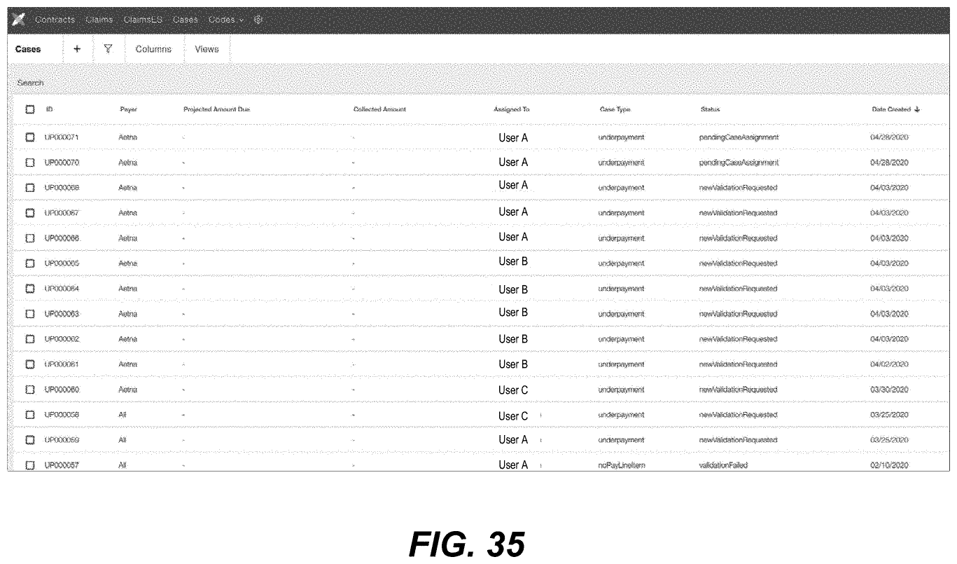

In the specific embodiment of FIG. 35, it is assumed that a user has used the system GUI to display a Cases page listing cases by identification number for a given account. This can take place, for example, by a user selecting by a user selecting a “Cases” tab at a top selection bar within the GUI. Alternatively, the user can input a keyboard shortcut to arrive at the same Cases page. Cases can be listed by case number, and additional information can be arranged in columns on this GUI page, which columns can include, for example, a payer, a projected amount due, a collected amount, who the case is assigned to, a case type, a status, and a date created. A regular user accessing this GUI page can see all cases in an account and get information regarding statuses. A developer accessing this GUI page can provide the user a grid view of the cases by account.

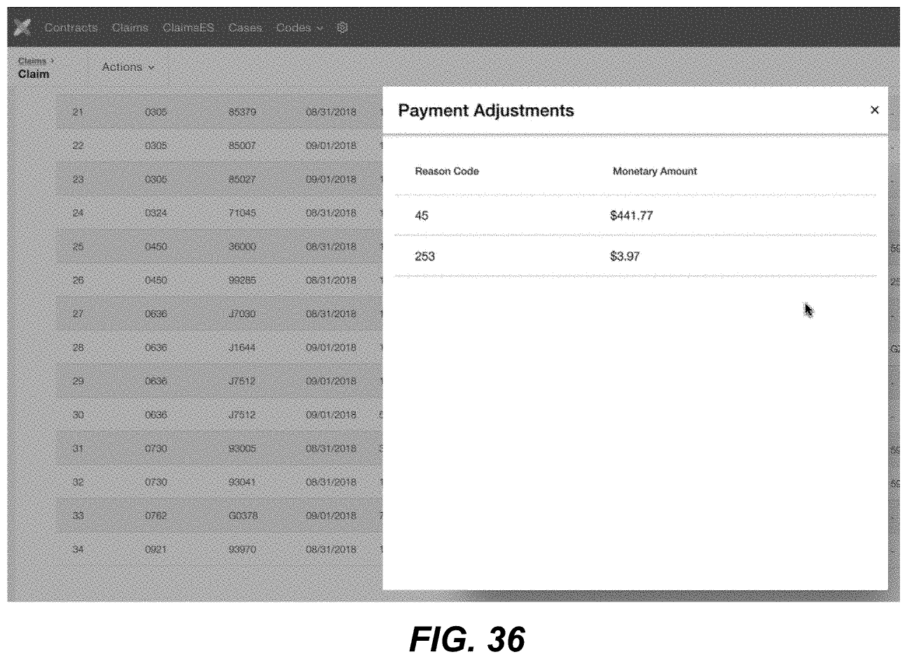

In the specific embodiment of FIG. 36, it is assumed that a user has used the system GUI to display a payment adjustments pop up box within a Claims page as noted above. This can take place, for example, by a user selecting a specific claim category within the GUI while on the Claims page. Alternatively, the user can input a keyboard shortcut to arrive at the same payment adjustments pop up box. Data and information on this payment adjustments pop up box can include, for example, reason codes and monetary amounts for payment adjustments that have been made for a selected claim

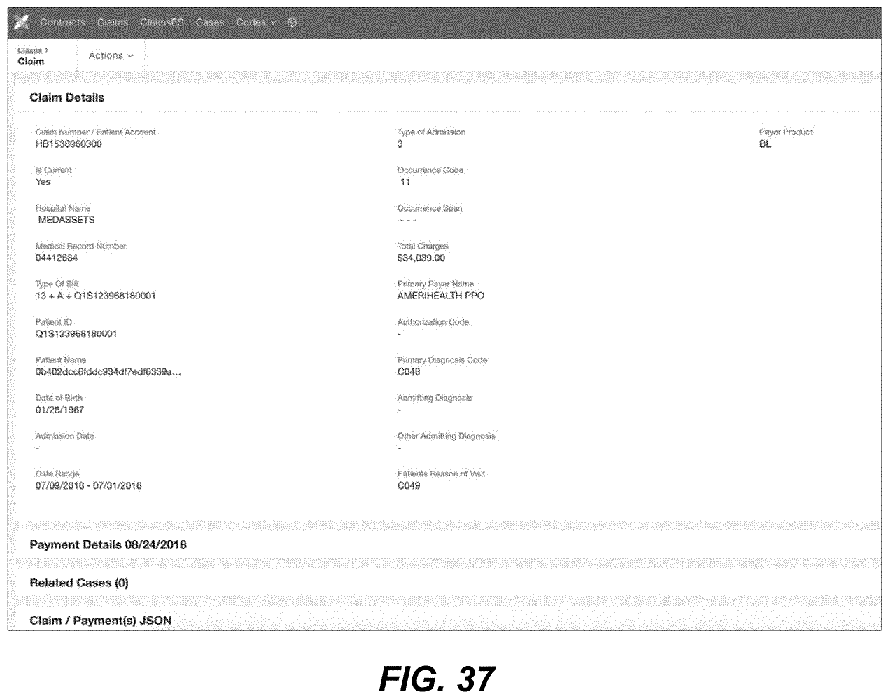

In the specific embodiment of FIG. 37, it is assumed that a user has used the system GUI to display a specific claim details page having pertinent details relating to a specific claim This can take place, for example, by a user selecting a specific claim within the GUI while on the Claims page. Alternatively, the user can input a keyboard shortcut to arrive at the same specific claim details page. Data and information on this GUI page can include, for example, claim number or patient account, current status, hospital name, medical record number, type of bill, patient ID, patient name (coded for HIPPA security purposes), patient data of birth, admission date, date range, type of admission, occurrence code, occurrence span, total charges, primary payor name, authorization code, primary diagnosis code, admitting diagnosis, other admitting diagnosis, and patient reason for visit, among other possible data items and selections. A regular user accessing this GUI page can see an expanded display of claim details for a given claim from the Claims page. A developer accessing this GUI page can allow users to get claim details by section.

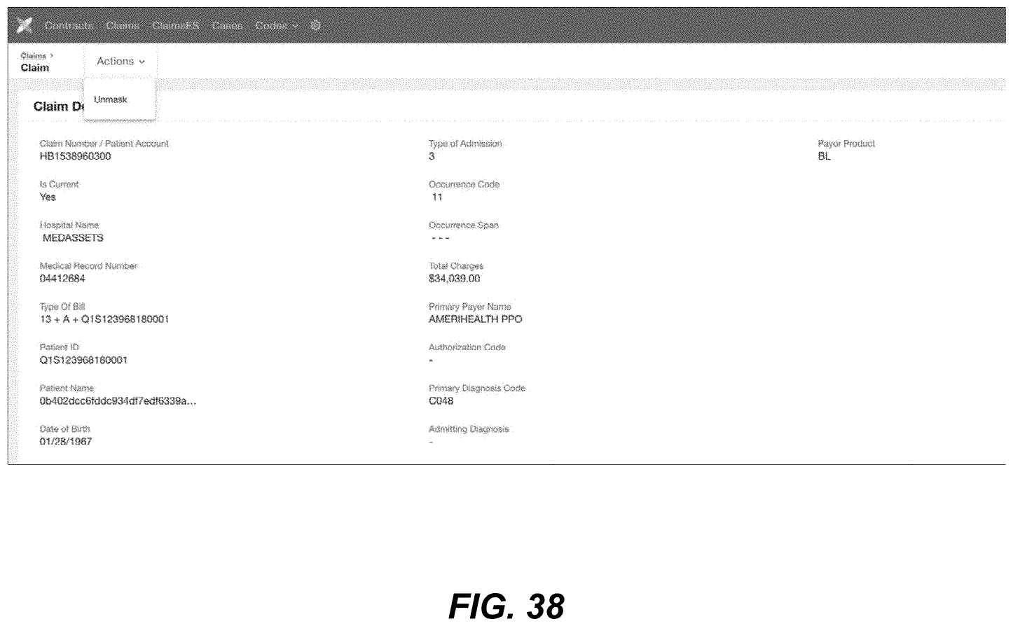

In the specific embodiment of FIG. 38, it is assumed that a user has used the system GUI to display the specific claim details page of FIG. 37 and has selected the action to unmask data. This added selection can take place, for example, by a user selecting an unmask option from an actions drop down menu at the top of the page. This selection can then unmask data on the page according to the security clearance of the user. A regular user accessing this GUI page option can unmask protected health information data (such as patient name, for example) per claim by access as provisioned. A developer accessing this GUI page option can log any user actions that see protected health information data in this manner.

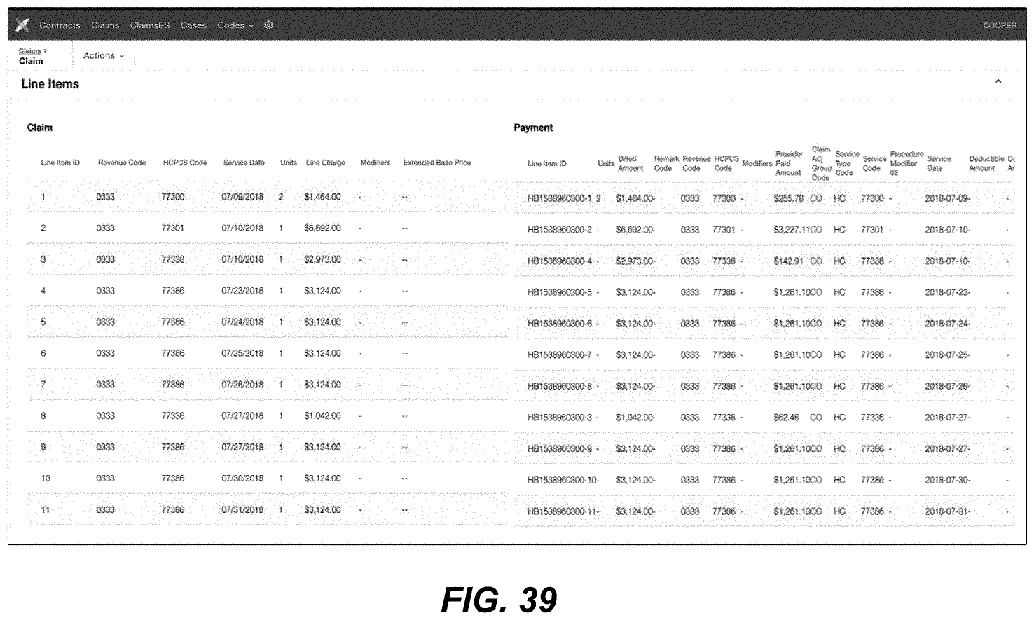

In the specific embodiment of FIG. 39, it is assumed that a user has used the system GUI to display a claim line items page section having all line items for a given claim, which can be an extended part of the specific claim details page of FIG. 37. This can take place, for example, by a user taking the same actions as above for FIG. 37 and then scrolling down the page to arrive at the claim line items page section within the GUI. Alternatively, the user can input a keyboard shortcut to arrive at the same claim line items page section. Data and information on this GUI page can be organized by lines for each claim line item and columns for information relating to each claim line item, which column information can include, for example, on the claim side a line item number, revenue code, HCPCS code, service data, units, line charge, modifiers, and extended base price, and on the payment side a line item ID, billed amount, remark code, revenue code, HCPCS code, modifiers, provider paid amount, claim adjustment group code, service type code, service code, procedure modifier, service date, deductible amount, and copayment amount, among other possible informational items.

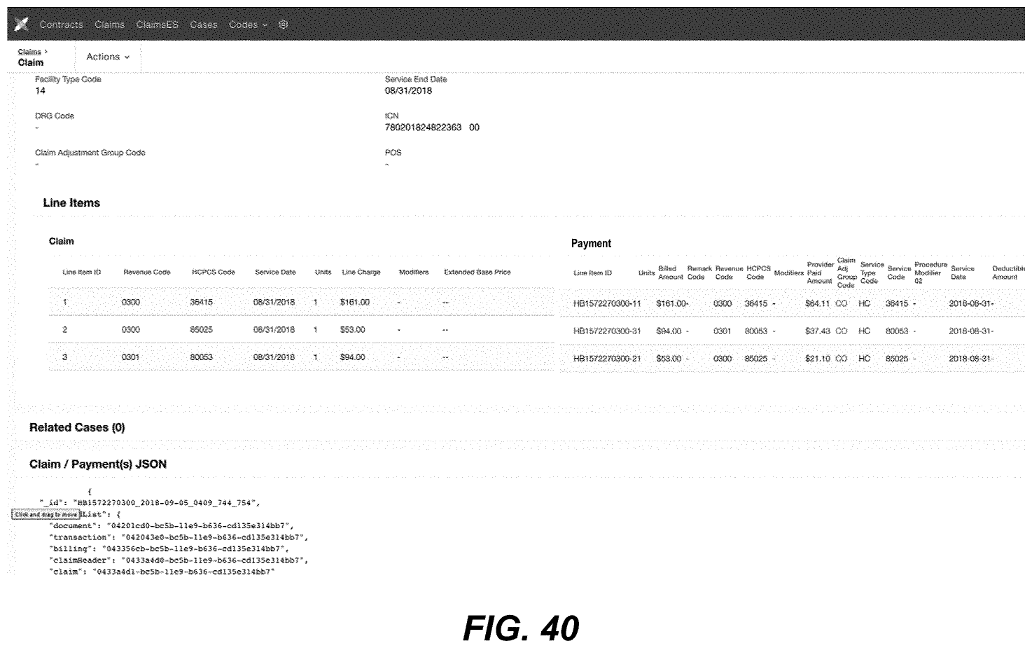

In the specific embodiment of FIG. 40, it is assumed that a user has used the system GUI to display an expanded specific claim page including claim information and claim line item information, as well as JSON data relating to this specific information. This can take place, for example, by a user selecting a specific claim within the GUI while on the Claims page and selecting an expanded specific JSON data view option, such as for an accordion GUI presentation. Alternatively, the user can input a keyboard shortcut to arrive at the same expanded presentation. Data and information on this expanded GUI page can include, for example, that which is listed above for FIGS. 37-40, as well as JSON data opened at the bottom of the page, which can be opened to further expand the page by selecting the hyperlink “Claim/Payment(s) JSON” located at the bottom of the page. In the event that the page is not large enough to display all of these items at once, a scroll feature can enable the user to scroll up and down the expanded page to view the desired information or data.

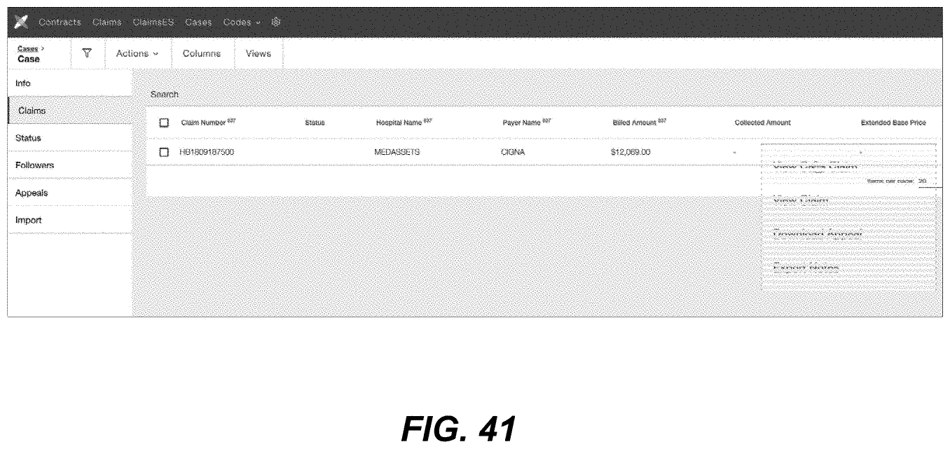

In the specific embodiment of FIG. 41, it is assumed that a user has used the system GUI to display a claims summary page for a given case. This can take place, for example, by a user selecting a “Cases” tab at a top selection bar within the GUI and then selecting “Claims” for a selected case from a drop down menu. Alternatively, the user can input a keyboard shortcut to arrive at the same claims summary page for a given case. Data and information on this GUI page can include, for example, a claim number, status, hospital name, payer name, billed amount, collected amount, and extended base price. A regular user accessing this GUI page can see all claims that are part of a case. A developer accessing this GUI page can give users a singular view to review the claims part of the case without the need to sort and filter.

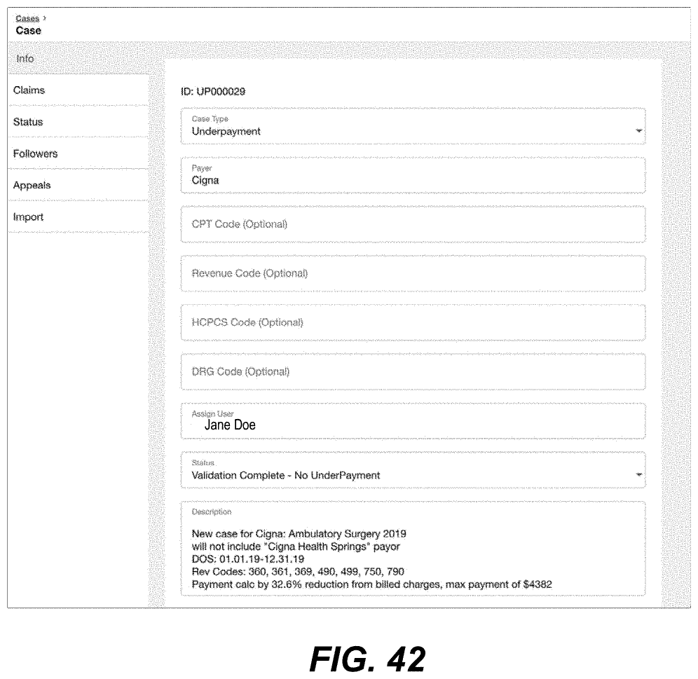

In the specific embodiment of FIG. 42, it is assumed that a user has used the system GUI to display a case info page for a given case. This can take place, for example, by a user selecting a “Cases” tab at a top selection bar within the GUI and then selecting “Info” for a selected case from a drop down menu. Alternatively, the user can input a keyboard shortcut to arrive at the same case info page. Data and information on this GUI page can include, for example, a case identifier, case type, payor, CPT code, revenue code, HCPCS code, DRG code, assigned user, status, and description for user notes. A regular user accessing this GUI page can retrieve key details on a case and can also perform CRUD operations as well. A developer accessing this GUI page can provide a view for users to maintain and view the case details.

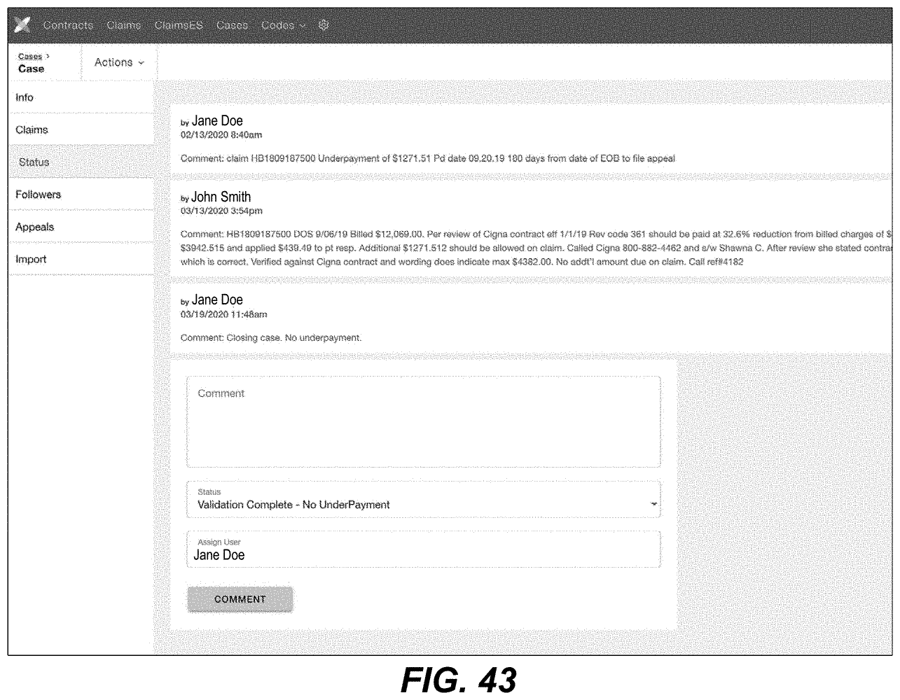

In the specific embodiment of FIG. 43, it is assumed that a user has used the system GUI to display a case status page for a given case. This can take place, for example, by a user selecting a “Cases” tab at a top selection bar within the GUI and then selecting “Status” for a selected case from a drop down menu. Alternatively, the user can input a keyboard shortcut to arrive at the same case info page. Data and information on this GUI page can include, for example, case status and various notes added by users. A regular user accessing this GUI page can add comments ad hoc to any case level details. A developer accessing this GUI page can allow users to free form add text that can be associated to a case.

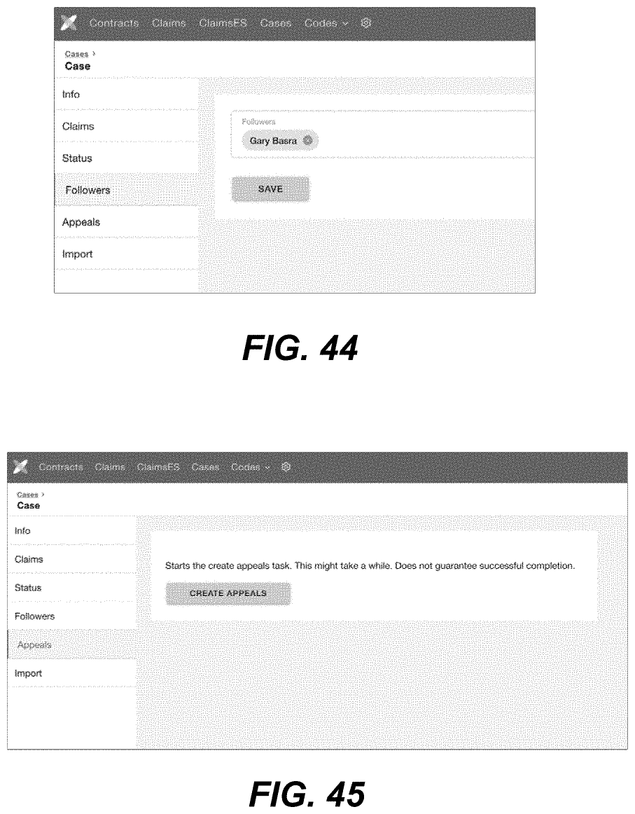

In the specific embodiment of FIG. 44, it is assumed that a user has used the system GUI to display a case followers page for a given case. This can take place, for example, by a user selecting a “Cases” tab at a top selection bar within the GUI and then selecting “Followers” for a selected case from a drop down menu. Alternatively, the user can input a keyboard shortcut to arrive at the same case info page. Data and information on this GUI page can include, for example, all users following the selected case. A regular user accessing this GUI page can add additional users to a case as followers. A developer accessing this GUI page can allow users to associate multiple authenticated users to a case.

In the specific embodiment of FIG. 45, it is assumed that a user has used the system GUI to display an appeals page for a given case. This can take place, for example, by a user selecting a “Cases” tab at a top selection bar within the GUI and then selecting “Appeals” for a selected case from a drop down menu. Alternatively, the user can input a keyboard shortcut to arrive at the same case appeals page. A regular user accessing this GUI page can select a Create Appeals button to automatically generate prefilled appeal forms for all of the claims in a case for appeals processing and correspondence. A developer accessing this GUI page can allow users to trigger the appeals letter generation that are used to further work the case with external entities. These forms can be predetermined and approved layouts.

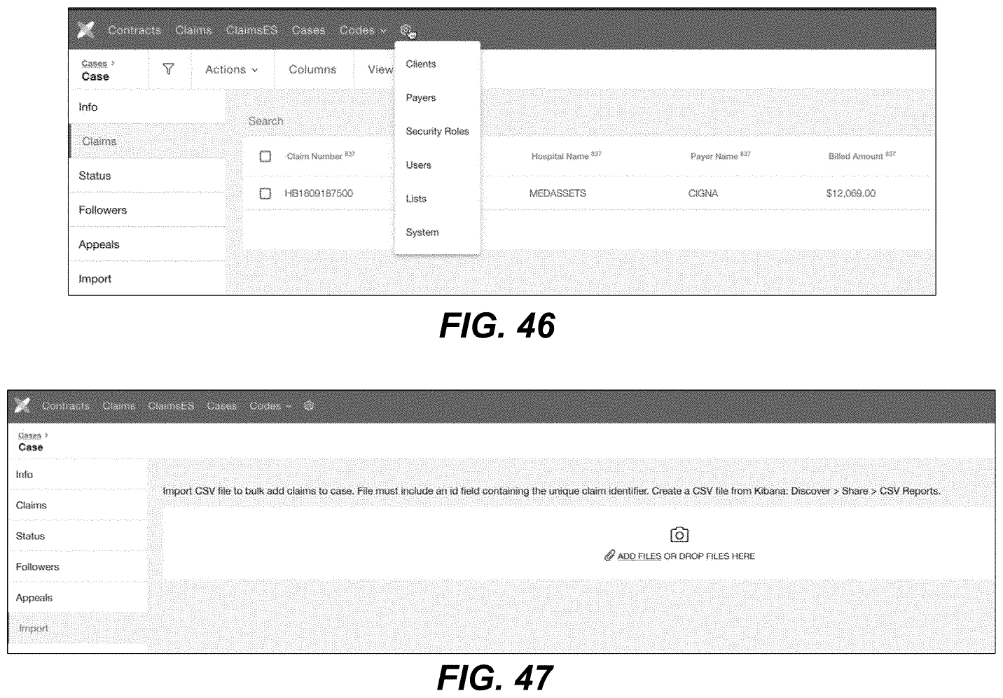

In the specific embodiment of FIG. 46, it is assumed that a user has used the system GUI to display utilities options. This can take place, for example, by a user selecting a utilities tab at a top selection bar within the GUI, which will then present a drop down menu of utilities options. Alternatively, the user can input a keyboard shortcut to arrive at the same utilities options. In various embodiments, access to these utilities options can be had from any page within the GUI. Utilities menu options can include, for example, clients, payers, security roles, users, lists, and system, among other possible options.

In the specific embodiment of FIG. 47, it is assumed that a user has used the system GUI to display an import page for a given case. This can take place, for example, by a user selecting a “Cases” tab at a top selection bar within the GUI and then selecting “Import” for a selected case from a drop down menu. Alternatively, the user can input a keyboard shortcut to arrive at the same case import page. A regular user accessing this GUI page can import external lists of claim numbers that can be assigned to the same case, which allows users to easily add more claims to an existing case. A developer accessing this GUI page can allow users to manage existing cases.

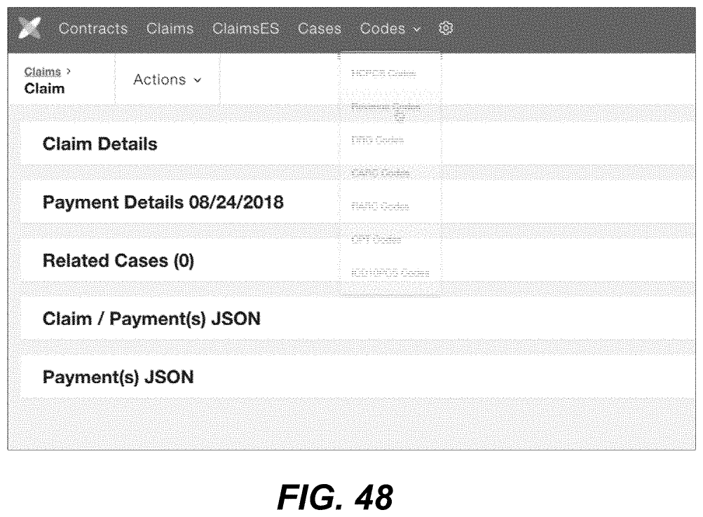

In the specific embodiment of FIG. 48, it is assumed that a user has used the system GUI to display standard codes options. This can take place, for example, by a user selecting a “Codes” tab at a top selection bar within the GUI, which will then present a drop down menu of different types of standard codes. Alternatively, the user can input a keyboard shortcut to arrive at the same menus of different types of codes. In various embodiments, access to these listed standard codes options can be had from any page within the GUI. Codes menu options can include, for example, HCPCS codes, revenue codes, DRG codes, CARC codes, RARC codes, CPT codes, and ICD10PCS codes.

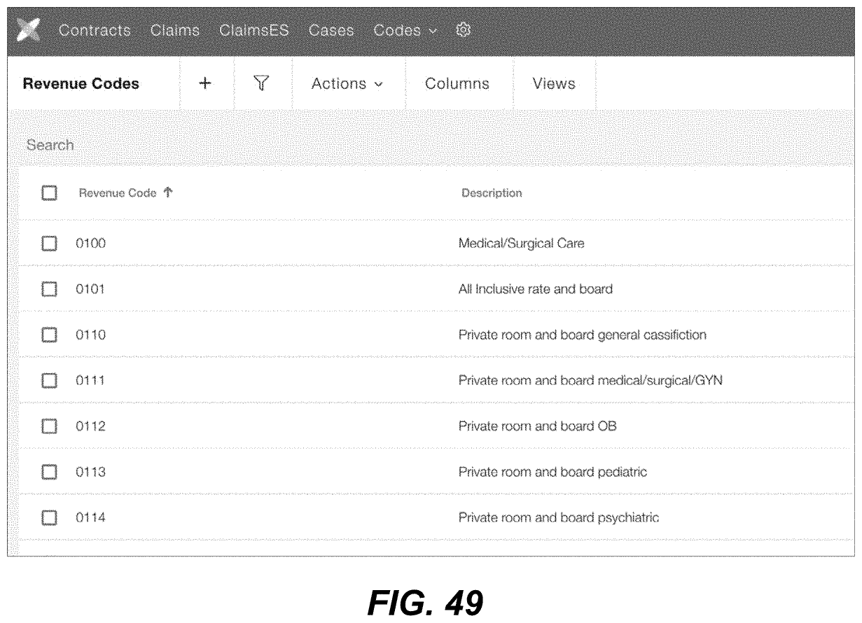

In the specific embodiment of FIG. 49, it is assumed that a user has used the system GUI to display a Codes page listing revenue codes. This can take place, for example, by a user selecting a “Codes” tab at a top selection bar within the GUI and then selecting revenue codes from a drop down menu from the “Codes” tab. Alternatively, the user can input a keyboard shortcut to arrive at the same revenue codes listing page. A regular user accessing this GUI page can view standard industry revenue codes, while a developer accessing this GUI page can CRUD operations revenue codes.

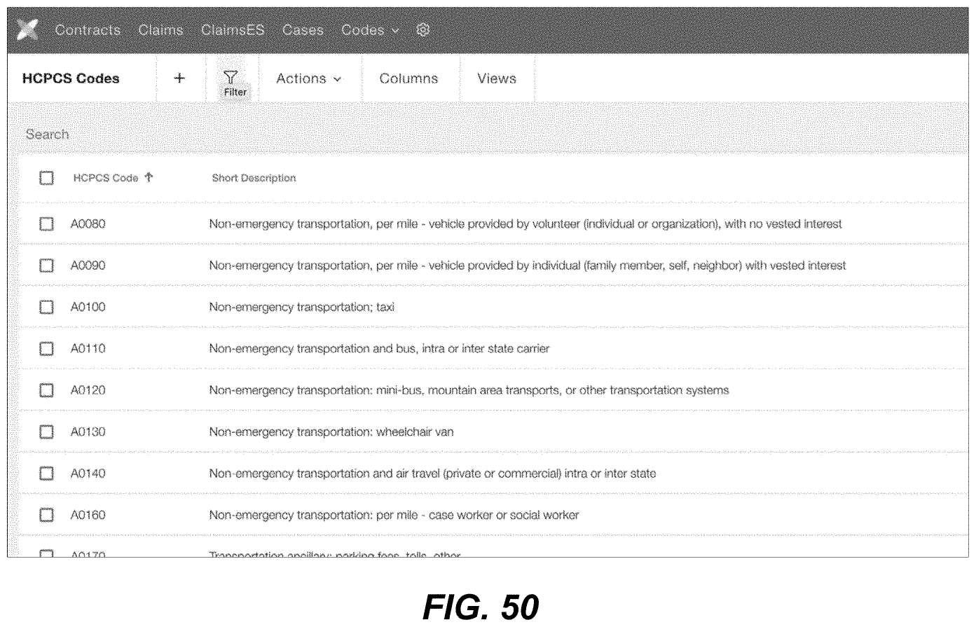

In the specific embodiment of FIG. 50, it is assumed that a user has used the system GUI to display a Codes page listing HCPCS codes. This can take place, for example, by a user selecting a “Codes” tab at a top selection bar within the GUI and then selecting HCPCS codes from a drop down menu from the “Codes” tab. Alternatively, the user can input a keyboard shortcut to arrive at the same HCPCS codes listing page. A regular user accessing this GUI page can view standard industry HCPCS codes, while a developer accessing this GUI page can CRUD operations HCPCS codes.

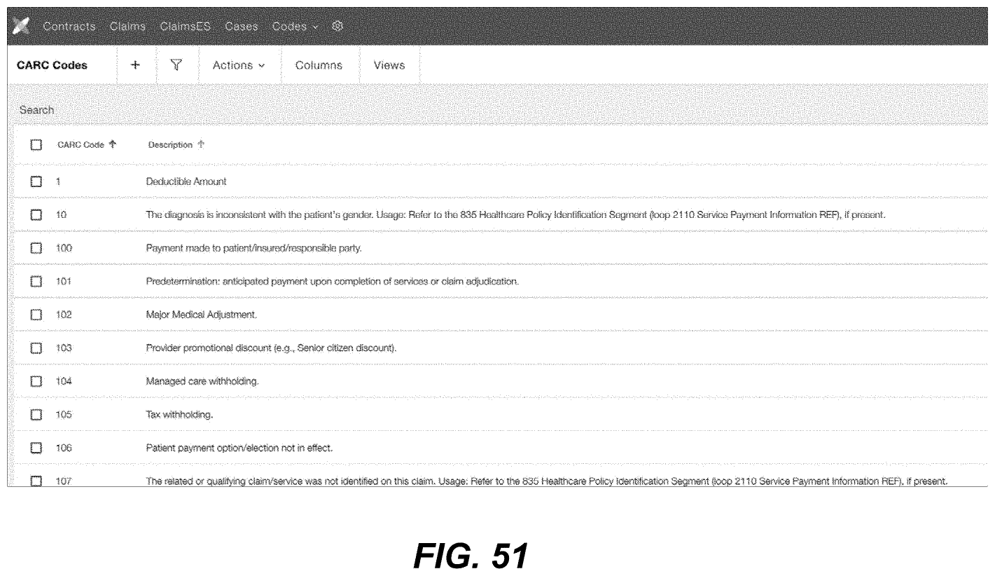

In the specific embodiment of FIG. 51, it is assumed that a user has used the system GUI to display a Codes page listing CARC codes. This can take place, for example, by a user selecting a “Codes” tab at a top selection bar within the GUI and then selecting CARC codes from a drop down menu from the “Codes” tab. Alternatively, the user can input a keyboard shortcut to arrive at the same CARC codes listing page. A regular user accessing this GUI page can view standard industry CARC codes, while a developer accessing this GUI page can CRUD operations CARC codes.

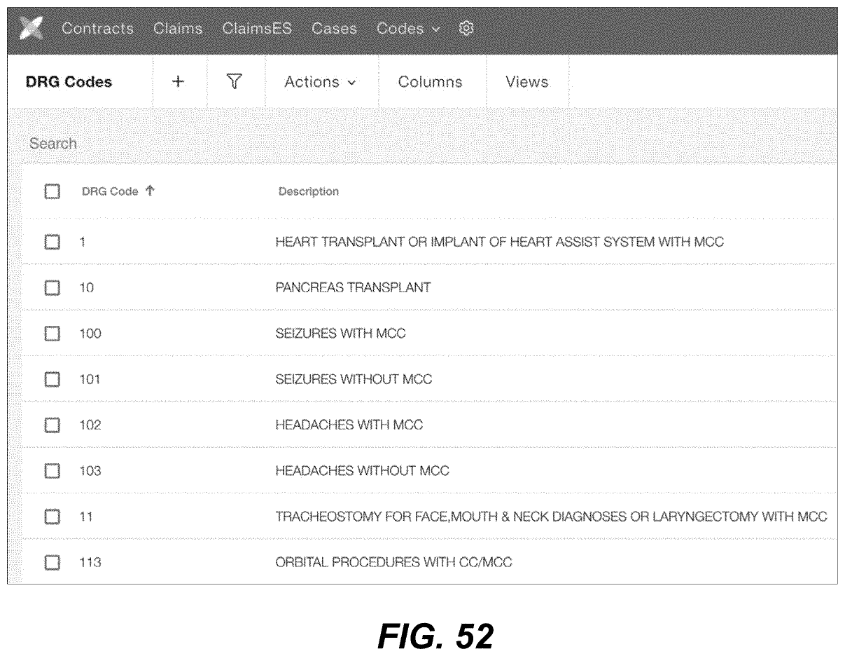

In the specific embodiment of FIG. 52, it is assumed that a user has used the system GUI to display a Codes page listing DRG codes. This can take place, for example, by a user selecting a “Codes” tab at a top selection bar within the GUI and then selecting DRG codes from a drop down menu from the “Codes” tab. Alternatively, the user can input a keyboard shortcut to arrive at the same DRG codes listing page. A regular user accessing this GUI page can view standard industry DRG codes, while a developer accessing this GUI page can CRUD operations DRG codes.

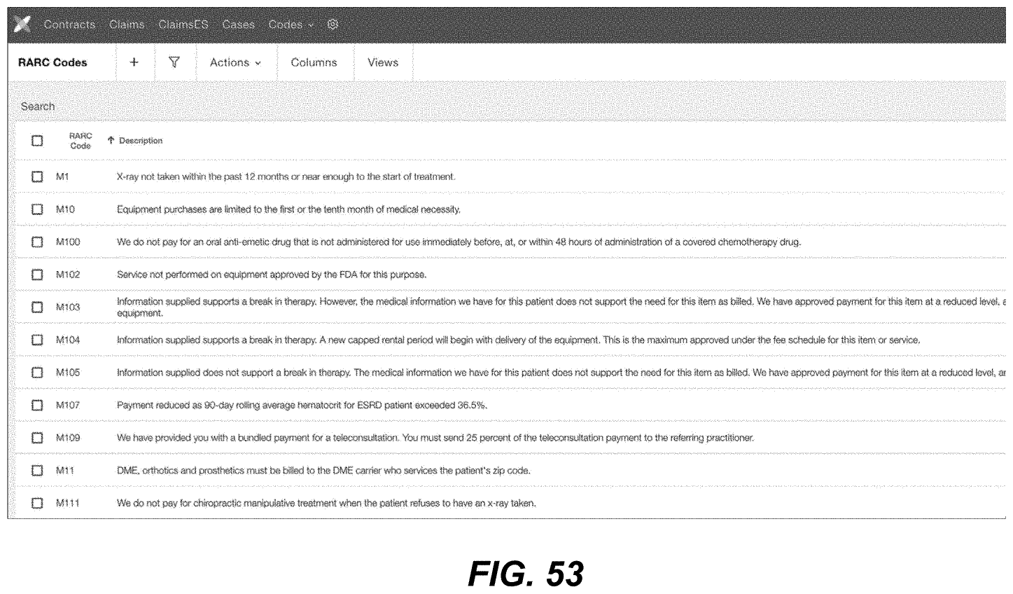

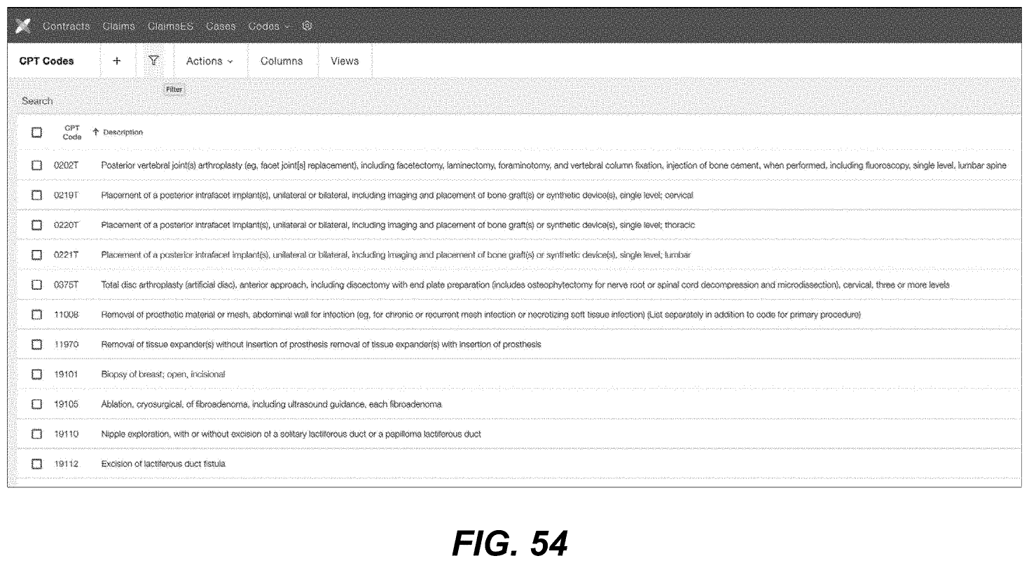

In the specific embodiment of FIG. 53, it is assumed that a user has used the system GUI to display a Codes page listing RARC codes. This can take place, for example, by a user selecting a “Codes” tab at a top selection bar within the GUI and then selecting RARC codes from a drop down menu from the “Codes” tab. Alternatively, the user can input a keyboard shortcut to arrive at the same RARC codes listing page. A regular user accessing this GUI page can view standard industry RARC codes, while a developer accessing this GUI page can CRUD operations RARC codes.

In the specific embodiment of FIG. 54, it is assumed that a user has used the system GUI to display a Codes page listing CPT codes. This can take place, for example, by a user selecting a “Codes” tab at a top selection bar within the GUI and then selecting CPT codes from a drop down menu from the “Codes” tab. Alternatively, the user can input a keyboard shortcut to arrive at the same CPT codes listing page. A regular user accessing this GUI page can view standard industry CPT codes, while a developer accessing this GUI page can CRUD operations CPT codes.

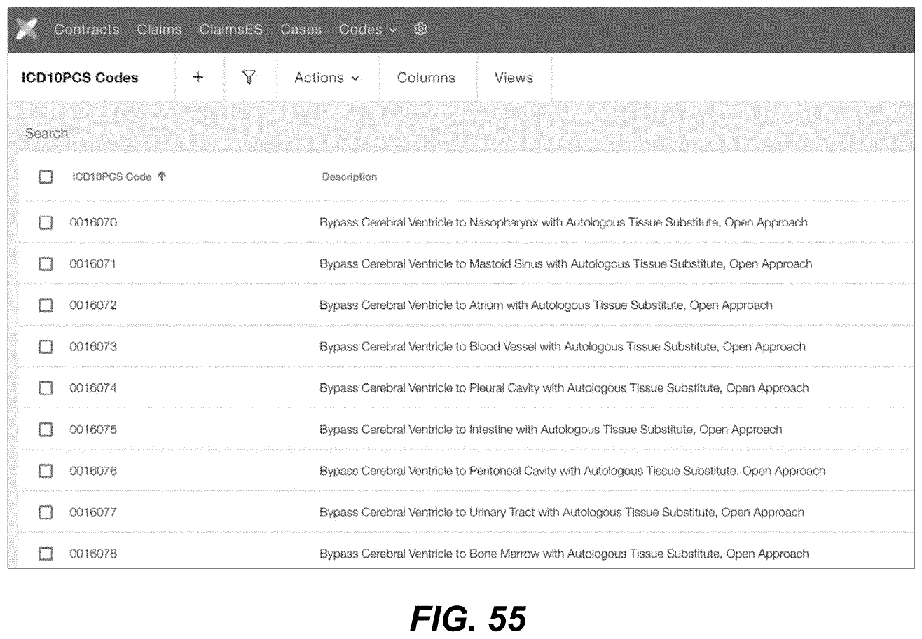

In the specific embodiment of FIG. 55, it is assumed that a user has used the system GUI to display a Codes page listing ICD10PCS codes. This can take place, for example, by a user selecting a “Codes” tab at a top selection bar within the GUI and then selecting ICD10PCS codes from a drop down menu from the “Codes” tab. Alternatively, the user can input a keyboard shortcut to arrive at the same ICD10PCS codes listing page. A regular user accessing this GUI page can view standard industry ICD10PCS codes, while a developer accessing this GUI page can CRUD operations ICD10PCS codes.

Those skilled in the art will readily appreciate that any of the systems and methods of the disclosure may include various computer and network related software and hardware, such as programs, operating systems, memory storage devices, data input/output devices, data processors, servers with links to data communication systems, wireless or otherwise, and data transceiving terminals, and may be a standalone device or incorporated in another platform, such as a portable computing device or electronic platform. In addition, the system of the disclosure may be provided at least in part on a personal computing device, such as home computer, laptop or mobile computing device through an online communication connection or connection with the Internet. Those skilled in the art will further appreciate that the precise types of software and hardware used are not vital to the full implementation of the methods of the disclosure so long as operators thereof are provided with useful access thereto as described herein.

The various aspects, embodiments, implementations or features of the described embodiments can be used separately or in any combination. Various aspects of the described embodiments can be implemented by software, hardware or a combination of hardware and software. Computer readable medium can be any data storage device that can store data which can thereafter be read by a computer system. Examples of computer readable medium include read-only memory, random-access memory, flash drives, USB drives, CD-ROMs, DVDs, magnetic disk drives, magnetic tape, optical data storage devices, and carrier waves. The computer readable medium can also be distributed over network-coupled computer systems so that the computer readable code is stored and executed in a distributed fashion.

Moreover, it will be appreciated that, via the use of specifically configured computer hardware and software, the problems which are solved and/or overcome by the various data analysis and information filtering techniques described herein are necessarily rooted in computer technology in order to overcome problems specifically arising in the realm of computer networks. For example, as described previously, numerous problems and limitations are typically encountered when attempting to use conventional manual based systems to implement data analysis and information filtering in claim reconciliation environments. Such problems and limitations specifically arise in the realm of computer networks, and the solutions to these data analysis and information filtering environment problems and limitations (e.g., as described herein) are necessarily rooted in computer technology.

Although the foregoing disclosure has been described in detail by way of illustration and example for purposes of clarity and understanding, it will be recognized that the above described disclosure may be embodied in numerous other specific variations and embodiments without departing from the spirit or essential characteristics of the disclosure. Certain changes and modifications may be practiced, and it is understood that the disclosure is not to be limited by the foregoing details, but rather is to be defined by the scope of the appended claims

What is claimed is:

1. A computer-implemented system for analyzing transactions between healthcare providers and insurance company payors, the system being deployed in a wide area network, the system comprising:
at least one processor;
at least one interface operable to provide a communication link to at least one network device;
non-transient memory;
a contract ingestion engine;
a claim automation engine;
a payment reconciliation engine;
the at least one processor being operable to execute a plurality of instructions stored in the memory for causing the contract ingestion engine to access a first contract data file, the first contract data file including contract information identifying a first healthcare provider and a first insurance payor,
the at least one processor being operable to execute a plurality of additional instructions stored in the memory for causing the contract ingestion engine to automatically process the first contract data file using optical character recognition, machine learning, and natural language processing;
wherein the processing of the first contract data file includes parsing the first contract data file to identify a first set of medical procedure identifier parameters identifying a first medical procedure;
wherein the processing of the first contract data file further includes parsing the first contract data file to identify a first set of contract payment rate parameters relating to the first medical procedure;
the at least one processor being operable to execute a plurality of additional instructions stored in the memory for causing the contract ingestion engine to generate, using the processed data of the first contract data file, a first contract rule record which includes the first set of medical procedure identifier parameters and includes the first set of contract payment rate parameters;
the at least one processor being operable to execute a plurality of additional instructions stored in the memory for causing the contract ingestion engine to populate a first contract rules database with information relating to the first contract rule, including the first set of medical procedure identifier parameters and the first set of contract payment rate parameters;
the at least one processor being operable to execute a plurality of additional instructions stored in the memory for causing the claim automation engine to access a first claim data file the first claim data file including a first portion of claim information identifying a first claim record, the first claim record including: a healthcare provider identifier identifying a second healthcare provider, an insurance payor identifier identifying a second insurance payor, and a patient identifier identifying a first patient;
the at least one processor being operable to execute a plurality of additional instructions stored in the memory for causing the claim automation engine to process the first portion of claim information to identify the healthcare provider identifier, the insurance payor identifier, and the patient identifier;
wherein the first portion of claim information includes a first portion of medical procedure identifier data identifying a first performed medical procedure associated with the first claim record;
the first portion of claim information further including a first portion of Payor paid amount data relating to a first Payor paid amount in connection with the first performed medical procedure;
the at least one processor being operable to execute a plurality of additional instructions stored in the memory for causing the claim automation engine to generate, using the processed first portion of claim information, a first processed claim record which includes normalized information identifying: the healthcare provider identifier, the insurance payor identifier, the patient identifier, the first performed medical procedure, and the first Payor paid amount;
the at least one processor being operable to execute a plurality of additional instructions stored in the memory for causing the claim automation engine to populate a first claims database with the first processed claim record;
the at least one processor being operable to execute a plurality of additional instructions stored in the memory for causing the payment reconciliation engine to access a second set of contract rule parameters associated with a second contracts rule, the second set of contract rule parameters including a second set of medical procedure identifier parameters identifying a second medical procedure, the second set of contract rule parameters further including a second set of contract payment rate parameters relating to the second medical procedure;
the at least one processor being operable to execute a plurality of additional instructions stored in the memory for causing the payment reconciliation engine to access information relating to a second processed claim record, the second processed claim record including a second portion of medical procedure identifier data identifying a second performed medical procedure associated with the second processed claim record, the second processed claim record further including a second portion of Payor paid amount data relating to a second Payor paid amount in connection with the second performed medical procedure;
the at least one processor being operable to execute a plurality of additional instructions stored in the memory for causing the payment reconciliation engine to determine, using the second set of contract rule parameters and the second processed claim record information, whether the second performed medical procedure associated with the second processed claim record is affected by the second set of contract rule parameters; payment reconciliation engine automation engine to calculate, in response to determining that the second performed medical procedure is affected by the second set of contract rule parameters, an expected contract payment amount for the second processed claim record, wherein the expected contract payment amount is calculated using the second set of contract rule parameters and the second processed claim record information;
the at least one processor being operable to execute a plurality of additional instructions stored in the memory for causing the payment reconciliation engine to compare the expected contract payment amount to the second Payor paid amount;
the at least one processor being operable to execute a plurality of additional instructions stored in the memory for causing the payment reconciliation engine to determine whether any payment discrepancy is detected between the expected contract payment amount and the second Payor paid amount;
the at least one processor being operable to execute a plurality of additional instructions stored in the memory for causing the payment reconciliation engine to generate, in response to detecting at least one payment discrepancy between the expected contract payment amount and the second Payor paid amount, a first portion of claim discrepancy data which includes information identifying the second processed claim record and includes claim payment discrepancy information relating to the at least one detected payment discrepancy associated with the second processed claim record; and
the at least one processor being operable to execute a plurality of additional instructions stored in the memory for causing the payment reconciliation engine to automatically initiate, in response to detecting at least one payment discrepancy between the expected contract payment amount and the second Payor paid amount, at least one operation to facilitate resolution of the at least one detected payment discrepancy associated with the second processed claim record.

2. The computer implemented system of claim 1 further comprising:
a first contract rules database;
a first claims database;
the system being further operable to cause the at least one processor to execute additional instructions to:
monitor the first contract rules database for at least one rule update event, the at least one rule update event relating to an occurrence of a new or modified rule record at the first contract rules database;
detect a rule update event relating to an identified contract rule record in the first contract rules database;
analyze the identified contract rule record to identify a set of contract rule parameters associated with the identified contract rule record; and
automatically instantiate and configure at least one automated agent process to perform payment reconciliation analysis on a first set of claim records using the identified set of contract rule parameters.

3. The computer implemented system of claim 1 being further operable to cause the at least one processor to execute additional instructions to:
cause the contract ingestion engine to populate a contract rules database with normalized information relating to the first contract rule.

4. The computer implemented system of claim 1 being further operable to cause the at least one processor to execute additional instructions to:
convert data from electronic data interchange (“EDI”) files to converted data of a different format.

5. The computer implemented system of claim 4, wherein the converted data is in JavaScript Object Notation (“JSON”) format.

6. The computer implemented system of claim 1 being further operable to cause the at least one processor to execute additional instructions to access a second contract data file, the second contract data file including contract information identifying a second healthcare provider and a second insurance company payor, wherein the first contract data file and the second contract data file are of disparate file formats.

7. The computer implemented system of claim 1 being further operable to cause the at least one processor to execute additional instructions to:
detect a rule update event relating to an identified contract rule record in a first contract rules database;
analyze the identified contract rule record to identify a third set of contract rule parameters associated with the identified contract rule record, the third set of contract rule parameters including a third set of medical procedure identifier parameters identifying a third medical procedure, the third set of contract rule parameters further including a third set of contract payment rate parameters relating to the third medical procedure;
automatically instantiate and configure at least one automated agent process to perform payment reconciliation analysis on a third set of claim records using the third set of contract rule parameters;
wherein the payment reconciliation analysis includes:
accessing information relating to a third processed claim record, the third processed claim record including a third portion of medical procedure identifier data identifying a third performed medical procedure associated with the third processed claim record, the third processed claim record further including a third portion of Payor paid amount data relating to a third Payor paid amount in connection with the third performed medical procedure;
determining, using the third set of contract rule parameters and the third processed claim record information, whether the third performed medical procedure associated with the third processed claim record is affected by the third set of contract rule parameters;
calculating, in response to determining that the third performed medical procedure is affected by the third set of contract rule parameters, a second expected contract payment amount for the third processed claim record, wherein the second expected contract payment amount is calculated using the third set of contract rule parameters and the third processed claim record information;
comparing the second expected contract payment amount to the third Payor paid amount;
determining whether any payment discrepancy is detected between the second expected contract payment amount and the third Payor paid amount; and
generating, in response to detecting at least one payment discrepancy between the second expected contract payment amount and the third Payor paid amount, a second portion of claim discrepancy data which includes information identifying the third processed claim record and includes claim payment discrepancy information relating to the at least one detected payment discrepancy associated with the third processed claim record.

8. The computer implemented system of claim 1 being further operable to cause the at least one processor to execute additional instructions to:
detect a first rule update event relating to a first identified contract rule record in a first contract rules database;
analyze the first identified contract rule record to identify a first set of contract rule parameters associated with the first identified contract rule record;
automatically instantiate and configure a first automated agent process to perform a first set payment reconciliation analysis operations on a first set of claim records using the first identified set of contract rule parameters;
detect a second rule update event relating to a second identified contract rule record in a second contract rules database;
analyze the second identified contract rule record to identify a second set of contract rule parameters associated with the second identified contract rule record;
automatically instantiate and configure a second automated agent process to perform a second set payment reconciliation analysis operations on a second set of claim records using the second identified set of contract rule parameters; and
wherein the first set payment reconciliation analysis operations and second set payment reconciliation analysis operations are performed substantially asynchronously and substantially concurrently.

9. The computer implemented system of claim 1, wherein new claim data added to the system after creation of the first contract rule is processed according to the first contract rule.

10. The computer implemented system of claim 1 being further operable to cause the at least one processor to execute additional instructions to:
separate flagged discrepancies by payor,
place the separated flagged discrepancies into categories, wherein the categories include one or more of denials, underpayments, unique short pays, systemic discrepancies of the same error type, or overcollections, and
rebill each payor according to its respective flagged discrepancies.

11. The computer implemented system of claim 1 wherein a significant number of rebilled flagged discrepancies are less than about $1000.

12. The computer implemented system of claim 1 being further operable to cause the at least one processor to execute additional instructions to automatically processes at least 50 million claim data records per hour.

13. The computer implemented system of claim 1 being further operable to cause the at least one processor to execute additional instructions to:
create and read existing contract rules when any changes occur, and
store the newly created contract rules in a contract rules database.

14. The computer implemented system of claim 1 being further operable to cause the at least one processor to execute additional instructions to:
generate a message to trigger a new process when a new contract rule is created, wherein the new process includes a stored function performable by automated agents,
place the message in a message queue, and
deliver the message to a third automated agent using a message broker when the message matures through the message queue, wherein the third automated agent is configured to execute instructions for performing a third payment reconciliation analysis involving the new contract rule.

15. A transaction analysis and payment reconciliation system, comprising:
one or more interfaces configured to receive inputs and provide outputs to a user, wherein the received inputs include contract data regarding transaction pricing and transaction data regarding specific transactions and related payments made;
a contract ingestion engine coupled to at least one of the one or more interfaces, wherein the contract ingestion engine is configured to import the contract data, normalize the imported contract data, parse the normalized contract data, and populate a contract rules database with the parsed contract data;
a transaction automation engine coupled to at least one of the one or more interfaces, wherein the transaction automation engine is configured to receive the transaction data, normalize the received transaction data, parse the normalized transaction data, and populate a transactions database with the parsed transaction data; and
a payment reconciliation engine coupled to at least one of the one or more interfaces, wherein the payment reconciliation engine is configured to receive parsed transaction data from the transaction instances database, correlate the received parsed transaction data with appropriate parsed contract data in the contract rules database, compare payments made in the received parsed transaction data with transaction pricing in the correlated parsed contract data, flag discrepancies between the payments made and the correlated transaction pricing, generate claim discrepancy data including at least claim identifications and discrepancy amounts, and provide access to the claim discrepancy data to at least one entity to facilitate recovery of the discrepancy amount.

16. The transaction analysis and asset recovery system of claim 15, further including:
the contract rules database; and
the transactions database.

17. The transaction analysis and asset recovery system of claim 15, wherein the transaction data includes service providers, service receivers, services performed, responsible payors, and payments made.

18. The transaction analysis and asset recovery system of claim 15, wherein the payment reconciliation engine is further configured to create a new rule when a discrepancy is flagged for which no rule exists and store the newly created rule in the contract rules database.

19. The transaction analysis and asset recovery system of claim 18, wherein the payment reconciliation engine is further configured to generate a message to trigger a new process when the new rule is created, the new process including comparing payments made in the received parsed transaction data with correlated transaction pricing in the parsed contract data for claims affected by the new rule, placing the message in a message queue, delivering the message using a message broker when the message matures through the message queue, and performing the new process asynchronously and substantially concurrently while processing new transaction data according to the new rule, the new transaction data being added to the system after creation of the new rule.

20. An automated system of analyzing transactions using a computing system, the system comprising:
importing contract data from a plurality of contracts between service providers and responsible payors;
normalizing the imported contract data into a format that is readily usable by the computing system;
parsing the normalized contract data into specific contract data portions that are readily usable by the computing system;
populate a contract rules database with the parsed contract data;
receive transaction data in a plurality of different formats from a plurality of different service providers, wherein the transaction data includes service providers, service receivers, services performed, responsible payors, and payments made;
normalizing the received transaction data into a format that is readily usable by the computing system;
parsing the normalized transaction data into specific transaction data portions that are readily usable by the computing system;
populate a transaction instances database with the parsed transaction data;
correlating a set of specific transaction data portions with appropriate specific contract data portions;
compare payments made in the set of specific transaction data portions with transaction pricing in the correlated specific contract data portions;
flag discrepancies between the payments made and the correlated transaction pricing, wherein at least some of the flagged discrepancies include recoverable assets;
generate claim discrepancy data including at least claim identifications and discrepancy amounts; and
provide access to the claim discrepancy data to at least one entity to facilitate recovery of the discrepancy amounts.

21. The system of claim 20, being further operable to the steps of:
creating a new rule when a discrepancy is flagged for which no rule exists;
storing the newly created rule in the contract rules database;
generate a message to trigger a new process when the new rule is created, wherein the new process includes recomparing payments made in the parsed transaction data with correlated transaction pricing in the parsed contract data for all claims affected by the new rule;
placing the message in a message queue;
delivering the message using a message broker when the message matures through the message queue; and
perform the new process asynchronously and simultaneously while processing new transaction data according to the new rule, wherein the new transaction data has been added to the computing system after creation of the new rule.

22. A computer implemented method for analyzing transactions between healthcare providers and insurance company payors, the method comprising causing at least one processor to execute a plurality of instructions stored in non-transient memory to:
cause a contract ingestion engine to access a first contract data file, the first contract data file including contract information identifying a first healthcare provider and a first insurance payor,
cause the contract ingestion engine to automatically process the first contract data file using optical character recognition, machine learning, and natural language processing;
wherein the processing of the first contract data file includes parsing the first contract data file to identify a first set of medical procedure identifier parameters identifying a first medical procedure;
wherein the processing of the first contract data file further includes parsing the first contract data file to identify a first set of contract payment rate parameters relating to the first medical procedure;
cause the contract ingestion engine to generate, using the processed data of the first contract data file, a first contract rule record which includes the first set of medical procedure identifier parameters and includes the first set of contract payment rate parameters;
cause the contract ingestion engine to populate a first contract rules database with information relating to the first contract rule, including the first set of medical procedure identifier parameters and the first set of contract payment rate parameters;
cause a claim automation engine to access a first claim data file the first claim data file including a first portion of claim information identifying a first claim record, the first claim record including: a healthcare provider identifier identifying a second healthcare provider, an insurance payor identifier identifying a second insurance payor, and a patient identifier identifying a first patient;
cause the claim automation engine to process the first portion of claim information to identify the healthcare provider identifier, the insurance payor identifier, and the patient identifier;
wherein the first portion of claim information includes a first portion of medical procedure identifier data identifying a first performed medical procedure associated with the first claim record;
the first portion of claim information further including a first portion of Payor paid amount data relating to a first Payor paid amount in connection with the first performed medical procedure;
cause the claim automation engine to generate, using the processed first portion of claim information, a first processed claim record which includes normalized information identifying: the healthcare provider identifier, the insurance payor identifier, the patient identifier, the first performed medical procedure, and the first Payor paid amount;
cause the claim automation engine to populate a first claims database with the first processed claim record;
cause a payment reconciliation engine to access a second set of contract rule parameters associated with a second contracts rule, the second set of contract rule parameters including a second set of medical procedure identifier parameters identifying a second medical procedure, the second set of contract rule parameters further including a second set of contract payment rate parameters relating to the second medical procedure;
cause the payment reconciliation engine to access information relating to a second processed claim record, the second processed claim record including a second portion of medical procedure identifier data identifying a second performed medical procedure associated with the second processed claim record, the second processed claim record further including a second portion of Payor paid amount data relating to a second Payor paid amount in connection with the second performed medical procedure;
cause the payment reconciliation engine to determine, using the second set of contract rule parameters and the second processed claim record information, whether the second performed medical procedure associated with the second processed claim record is affected by the second set of contract rule parameters; payment reconciliation engine automation engine to calculate, in response to determining that the second performed medical procedure is affected by the second set of contract rule parameters, an expected contract payment amount for the second processed claim record, wherein the expected contract payment amount is calculated using the second set of contract rule parameters and the second processed claim record information;
cause the payment reconciliation engine to compare the expected contract payment amount to the second Payor paid amount;
cause the payment reconciliation engine to determine whether any payment discrepancy is detected between the expected contract payment amount and the second Payor paid amount;
cause the payment reconciliation engine to generate, in response to detecting at least one payment discrepancy between the expected contract payment amount and the second Payor paid amount, a first portion of claim discrepancy data which includes information identifying the second processed claim record and includes claim payment discrepancy information relating to the at least one detected payment discrepancy associated with the second processed claim record; and
cause the payment reconciliation engine to automatically initiate, in response to detecting at least one payment discrepancy between the expected contract payment amount and the second Payor paid amount, at least one operation to facilitate resolution of the at least one detected payment discrepancy associated with the second processed claim record.
